(19) United States
(12) Patent Application Publication
(10) Pub. No.: US 2024/0108738 A1
(43) Pub. Date: Apr. 4, 2024

(54) BICYCLIC PEPTIDE LIGANDS SPECIFIC FOR NK CELLS

(71) Applicant: Bicycle Tx Limited, Cambridge (GB)

(72) Inventors: Nicholas KEEN, Carlisle, MA (US); Gemma MUDD, Cambridge (GB); Fay DUFORT, Cambridge (GB); Katie GAYNOR, Cambridge (GB); Liuhong CHEN, Cambridge (GB); Phil BRANDISH, Needham, MA (US); Cris LEITHEISER, Cambridge (GB); Sandra UHLENBROICH, Cambridge (GB); Liz REPASH, Cambridge (GB); Kevin MCDONNELL, Lexington, MA (US)

(21) Appl. No.: 18/271,353

(22) PCT Filed: Jan. 10, 2022
(86) PCT No.: PCT/GB2022/050043

§ 371 Date: Jul. 7, 2023

Related U.S. Application Data
(60) Provisional application No. 63/135,339, filed on Jan. 8, 2021.
(60) Provisional application No. 63/262,597, filed on Oct. 15, 2021.

(51) Int. Cl. A61K 47/64; A61P 37/06; C07K 7/08; C07K 14/00

(52) U.S. Cl. CPC A61K 47/64; A61P 37/06; C07K 7/08; C07K 14/001; A61K 38/00

(57) ABSTRACT

The present invention relates to polypeptides which are covalently bound to molecular scaffolds such that two or more peptide loops are subtended between attachment points to the scaffold. In particular, the invention describes peptides which bind to natural killer (NK) cells. The invention also includes multimeric binding complexes comprising at least two of said bicyclic peptide ligands. The invention also includes pharmaceutical compositions comprising said peptide ligands or said multimeric binding complexes and the use of said peptide ligands, multimeric binding complexes and pharmaceutical compositions in preventing, suppressing or treating a disease or disorder mediated by natural killer (NK) cells, such as inflammatory disorders, autoimmune disease and cancer.

### FIELD OF THE INVENTION

The present invention relates to polypeptides which are covalently bound to molecular scaffolds such that two or more peptide loops are subtended between attachment points to the scaffold. In particular, the invention describes peptides which bind to natural killer (NK) cells. The invention also includes multimeric binding complexes comprising at least two of said bicyclic peptide ligands. The invention also includes pharmaceutical compositions comprising said peptide ligands or said multimeric binding complexes and the use of said peptide ligands, multimeric binding complexes and pharmaceutical compositions in preventing, suppressing or treating a disease or disorder mediated by natural killer (NK) cells, such as inflammatory disorders, autoimmune disease and cancer.

### BACKGROUND OF THE INVENTION

Cyclic peptides are able to bind with high affinity and specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat. Rev. Drug. Discov. 7(7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296(5565), 151-5) or the cyclic peptide inhibitor upain-1 binding to urokinase-type plasminogen activator (603 Å²; Zhao et al. (2007), J. Struct. Biol. 160(1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J. Med. Chem. 41(11), 1749-51). The favourable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example 1,1′,1″-(1,3,5-triazinane-1,3,5-triyl)triprop-2-en-1-one (TATA) (Heinis et al. (2014) Angewandte Chemie, International Edition 53(6) 1602-1606).

Phage display-based combinatorial approaches have been developed to generate and screen large libraries of bicyclic peptides to targets of interest (Heinis et al. (2009), Nat. Chem. Biol. 5(7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to a first aspect of the invention, there is provided a peptide ligand specific for natural killer (NK) cells comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

According to a yet further aspect of the invention, there is provided a pharmaceutical composition comprising a peptide ligand as defined herein in combination with one or more pharmaceutically acceptable excipients.

According to a further aspect of the invention, there is provided a peptide ligand or pharmaceutical composition as defined herein for use in preventing, suppressing or treating a disease or disorder mediated by natural killer (NK) cells.

### BRIEF DESCRIPTION OF THE FIGURES

FIG. 1: Binding of Alexa Fluor® 488 labelled NKp46 bicyclic peptide dimers to CHO-K1/NKp46 or CHO-K1 parental NKp46 null cell lines. (A) illustrates that Alexa Fluor® 488-tagged NKp46 bicyclic peptide dimers bind in a dose-dependent manner to NKp46 expressing cells. No binding is observed in the CHO-K1 parental cell line (B), which is null for NKp46. Multiple AF488-NKp46 dimers were evaluated, shown in Table 4. BCY15665 is a non-binding bicyclic peptide dimer, comprised of all D-amino acids.

FIG. 2: Binding of Alexa Fluor® 488 labelled NKp46 bicyclic peptide dimers to CD56+(NK cells) or CD56− cells from human PBMCs. (A) Binding of Alexa Fluor® 488 labelled NKp46 bicyclic peptide dimers to PBMC populations that express CD56 (NK cell population). (B) Cells in the PBMC population that do not express CD56 and that are CD3+(T-cell population) elicit no binding. Data from a single representative PBMC donor (n=4). BCY15665 is a non-binding bicyclic peptide dimer, comprised of all D-amino acids.

FIG. 3: IgG Competition Assay with C-terminally biotinylated human CD16a F176 variant (F Variant) using BCY15914 (A), BCY15915 (B) and BCY15916 (C).

FIG. 4: IgG Competition Assay with N-terminally biotinylated human CD16a V176 variant (V Variant) using BCY15914 (A), BCY15915 (B) and BCY15916 (C).

### DETAILED DESCRIPTION OF THE INVENTION

According to a first aspect of the invention, there is provided a peptide ligand specific for natural killer (NK) cells comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

Natural killer (NK) cells are members of the innate immune system representing a small fraction of peripheral blood mononuclear cells. As frontline responders, these immune cells detect and eliminate unhealthy cells and bridge the innate immune response to the adaptive immune response. Due to their inherent properties, NK cells are an excellent candidate to enhance the therapeutic tools in immune oncology and autoimmunity.

NK cells are responsible for immune surveillance conducted through a variety of inhibitory and activating receptors. These activating and inhibitory receptors on the NK cellular surface are a complex means through which the activity of NK cells is kept in balance in healthy individuals. NK cells recognize the MHC class I molecules on the surface of healthy cells and are restrained through inhibitory receptors from eliminating these healthy cells. In times of stress, infection, or transformation, NK cells recognize the unhealthy cells through the loss of MHC class I on the cell surface and the induction of NK cell receptor ligands which bind to activating receptors. The recognition of non-self by the NK cells elicits a cytotoxic response, a release of cytokines and cytotoxic molecules for the elimination of the unhealthy cells.

NK cell activity is by a complex mechanism that involves both activating and inhibitory signals. Multiple reports have provided evidence for a central role of NK cell receptors in natural cytotoxicity and usefulness in the treatment of cancer. There is an unmet need for further understanding and enhancement of NK cell mediated recognition and killing of tumor cells. Reports suggest tumor cells utilize many mechanisms to reduce NK activity, and that NK cell presence and efficacy is associated with favorable prognosis in patients (Pasero et al. (2015) Oncotarget 6(16), 14360-14373, Stringaris et al. (2014) Haematologica 99(5), 836-847). It is through therapeutic intervention that one may harness the potential NK cells may play in mediating an immune response to combat cancer and autoimmune diseases.

NKp46 Binding Bicyclic Peptides

In one embodiment, the bicyclic peptide is specific for (i.e. binds to) a natural cytotoxicity receptor present on the NK cell surface. In a further embodiment, the bicyclic peptide is specific for (i.e. binds to) a natural cytotoxicity receptor selected from NKp30, NKp44 and NKp46. In a yet further embodiment, the bicyclic peptide is specific for (i.e. binds to) NKp46.

The natural cytotoxicity receptors (NCR) are a family of

stimulatory receptors expressed on the NK cell surface that elicit NK activation and cell-mediated cytotoxicity. The NCR family consists of three members, NKp30, NKp44, and NKp46. Although the cellular ligand for NKp46 is unknown, a role for NKp46 in antitumor immunity has been shown. Viral antigen-mediated NKp46 activation of NK cells results in tumor rejection (Chinnery et al. 2012). Upon interaction with its ligand, the NKp46 receptor triggers NK cells to induce directed cytotoxicity, illustrated by the use of anti-NKp46 blocking antibodies inhibiting the ability of NK cells to lyse targets (Arnon et al. 2004). The amount of NCR expression on the NK cell surface also increases NK cytotoxicity. A strong correlation between the density of NCR expression and the ability of NK cells to kill target cells, including a wide variety of tumor cells, has been identified (Moretta et al. 2006). In AML and in cervical cancer and precursor lesions, the insufficient amount of NCR or NCR ligands rendered tumor cells resistant to NK cytotoxicity (Costello et al. 2002, Garcia-Iglesias et al. 2009). In many solid tumors, NK cells are downregulated by the tumor microenvironment, among which include the tumor shedding of NCR ligands and immune editing, which prevent NK cells' ability to recognize, infiltrate, and kill the tumor cells (Nayyar 2019, Stojanovic et al. 2011, Sordo-Bahamonde et al. 2020, Watanabe et al. 2010, Izawa et al. 2011, Koo et al. 2013, Sun et al. 2015, Hasmim et al. 2015, Han et al. 2018). Stringaris et al. (2014) reported downregulation of NKp46, upregulation of NK cell inhibitory receptor NKG2A and low cytotoxic capacity of NK cells from AML patients. Furthermore, in solid cancer such as prostate cancer, there was reported a decreased expression of several activating receptors (CD16, NKp30, NKp46, NKG2D and DNAM-1), and an increase in the inhibitory receptor CD85j (Pesaro et al. 2016). In contrast, Gautheir et al. (2019) has identified NKp46 as a good candidate for the targeting of an activating receptor on NK cells in cancer, demonstrating no statistically significant downregulation of NKp46 in the periphery in SCCHN, breast, liver, lung, kidney, and metastatic melanoma cancer patients. Additionally, in multiple solid tumors sustained NKp46 expression, associated with the downregulation of other activating receptors, such as NKG2D, NKp30, and NKp44, and low CD16 expression on tumor infiltrating lymphocytes has been reported for cancers, such as acute myeloid leukemia, breast cancer, and lung carcinoma (Fauriat et al. 2007, Mamessier et al. 2011, Platonova et al. 2011, Levi et al. 2015, Kim et al. 2010, MacFarlane et al. 2017). Therefore, NKp46 has shown to be a specific NK surface marker suitable for therapeutic application to identify and targeting NK cells to tumors.

In a further embodiment, said loop sequences comprise 4, 5, 6 or 7 amino acids.

In one embodiment, said loop sequences comprise three cysteine residues separated by two loop sequences the first of which consists of 5 amino acids and the second of which consists of 4 amino acids.

In a further embodiment, said loop sequences comprise three cysteine residues separated by two loop sequences the first of which consists of 5 amino acids and the second of which consists of 4 amino acids and the bicyclic peptide ligand comprises an amino acid sequence which is selected from:

| (SEQ ID NO: 1) | |
|---|---|
| | CᵢNLQKPCᵢᵢMKYPCᵢᵢᵢ; |
| | and |
| (SEQ ID NO: 2) | |
| | CᵢNLQNPCᵢᵢMKFPCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 5 amino acids and the second of which consists of 4 amino acids, the molecular scaffold is TATA and the bicyclic peptide ligand additionally comprises N- and/or C-terminal additions and comprises an amino acid sequence which is selected from:
A-(SEQ ID NO: 1)-A-Sar₆-KFI (herein referred to as BCY14654);
A-(SEQ ID NO: 1)-A (herein referred to as BCY14456);
A-(SEQ ID NO: 2)-A-Sar₆-KFI (herein referred to as BCY15125);
A-(SEQ ID NO: 2)-A (herein referred to as BCY15102); and
A-(SEQ ID NO: 2)-A-[K(PYA)] (herein referred to as BCY18004),

wherein PYA represents pentynoic acid, Sar represents sarcosine and FI represents fluorescein.

In a still yet further embodiment, said loop sequences comprise three reactive groups separated by two loop sequences the first of which consists of 5 amino acids and the second of which consists of 4 amino acids, the molecular scaffold is TATA and the bicyclic peptide ligand additionally comprises N- and/or C-terminal additions and comprises an amino acid sequence which is selected from:
A-(SEQ ID NO: 1)-A-Sar₆-KFI (herein referred to as BCY14654); and
A-(SEQ ID NO: 2)-A-Sar₆-KFI (herein referred to as BCY15125),

wherein Sar represents sarcosine and FI represents fluorescein.

In an alternative embodiment, said loop sequences comprise three cysteine residues separated by two loop sequences the first of which consists of 5 amino acids and the second of which consists of 6 amino acids.

In a further embodiment, said loop sequences comprise three cysteine residues separated by two loop sequences the first of which consists of 5 amino acids and the second of which consists of 6 amino acids and the bicyclic peptide ligand comprises an amino acid sequence which is selected from:

| (SEQ ID NO: 3) | |
|---|---|
| | CᵢNLQAPCᵢᵢMQTGKVCᵢᵢᵢ; |
| | (SEQ ID NO: 4; herein |
| | referred to as BCY18295 |
| | when complexed with TATA) |
| | CᵢNLQAPCᵢᵢMLTGKVCᵢᵢᵢ; |
| (SEQ ID NO: 63) | |
| | CᵢNLQQACᵢᵢLHTGKVCᵢᵢᵢ; |
| (SEQ ID NO: 64) | |
| | CᵢNLQAACᵢᵢMLTGKVCᵢᵢᵢ; |
| (SEQ ID NO: 65) | |
| | CᵢNLQAPCᵢᵢMATGKVCᵢᵢᵢ; |
| (SEQ ID NO: 66) | |
| | CᵢNLQAPCᵢᵢMLAGKVCᵢᵢᵢ; |
| (SEQ ID NO: 67) | |
| | CᵢNLQAPCᵢᵢMLTAKVCᵢᵢᵢ; |
| (SEQ ID NO: 68) | |
| | CᵢNLQAPCᵢᵢMLTGAVCᵢᵢᵢ; |
| (SEQ ID NO: 69) | |
| | CᵢNLQAPCᵢᵢMLTGKACᵢᵢᵢ; |
| (SEQ ID NO: 70) | |
| | CᵢN[tBuAla]QAPCᵢᵢMLTGKVCᵢᵢᵢ; |
| (SEQ ID NO: 71) | |
| | CᵢNLQA[Sar]CᵢᵢMLTGKVCᵢᵢᵢ; |
| (SEQ ID NO: 72) | |
| | CᵢNLQA[Cis-HyP]CᵢᵢMLTGKVCᵢᵢᵢ; |
| (SEQ ID NO: 73) | |
| | CᵢNLQA[HyP]CᵢᵢMLTGKVCᵢᵢᵢ; |
| (SEQ ID NO: 74) | |
| | CᵢNLQA[NMeAla]CᵢᵢMLTGKVCᵢᵢᵢ; |
| (SEQ ID NO: 75) | |
| | CᵢNLQAPCᵢᵢLLTGKVCᵢᵢᵢ; |
| (SEQ ID NO: 76) | |
| | CᵢNLQAPCᵢᵢ[Nle]LTGKVCᵢᵢᵢ; |
| (SEQ ID NO: 77) | |
| | CᵢNLQAPCᵢᵢ[Cba]LTGKVCᵢᵢᵢ; |
| (SEQ ID NO: 78) | |
| | CᵢNLQAPCᵢᵢMETGKVCᵢᵢᵢ; |
| (SEQ ID NO: 79) | |
| | CᵢNLQAPCᵢᵢMFTGKVCᵢᵢᵢ; |
| (SEQ ID NO: 80) | |
| | CᵢNLQAPCᵢᵢMYTGKVCᵢᵢᵢ; |
| (SEQ ID NO: 81) | |
| | CᵢNLQAPCᵢᵢM[Cba]TGKVCᵢᵢᵢ; |
| (SEQ ID NO: 82) | |
| | CᵢNLQAPCᵢᵢM[tBuAla]TGKVCᵢᵢᵢ; |
| (SEQ ID NO: 83) | |
| | CᵢNLQAPCᵢᵢM[Cha]TGKVCᵢᵢᵢ; |
| (SEQ ID NO: 84) | |
| | CᵢNLQAPCᵢᵢMLT[dA]KVCᵢᵢᵢ; |
| (SEQ ID NO: 85) | |
| | CᵢNLQAPCᵢᵢMLTGRVCᵢᵢᵢ; |
| (SEQ ID NO: 86) | |
| | CᵢNLQAPCᵢᵢMLTG[Agb]VCᵢᵢᵢ; |
| (SEQ ID NO: 87) | |
| | CᵢNLQAPCᵢᵢMLTGK[tBuGly]Cᵢᵢᵢ; |
| (SEQ ID NO: 88) | |
| | CᵢNLQAPCᵢᵢMLTGK[Chg]Cᵢᵢᵢ; |
| (SEQ ID NO: 89) | |
| | CᵢNLQAPCᵢᵢMLTGK[Cbg]Cᵢᵢᵢ; |
| (SEQ ID NO: 90) | |
| | CᵢNLQAPCᵢᵢMLTG[HArg]VCᵢᵢᵢ; |
| (SEQ ID NO: 91) | |
| | CᵢNLQA[Aze]CᵢᵢMLTGKVCᵢᵢᵢ; |
| (SEQ ID NO: 92) | |
| | CᵢNLQA[Pip]CᵢᵢMLTGKVCᵢᵢᵢ; |
| (SEQ ID NO: 93) | |
| | CᵢNLQAPCᵢᵢMVTGKVCᵢᵢᵢ; |
| (SEQ ID NO: 94) | |
| | CᵢNLQAPCᵢᵢM[Nle]TGKVCᵢᵢᵢ; |
| (SEQ ID NO: 95) | |
| | CᵢNLQAPCᵢᵢM[Nva]TGKVCᵢᵢᵢ; |
| (SEQ ID NO: 96) | |
| | CᵢNLQAPCᵢᵢMLTGK[Allolle]Cᵢᵢᵢ; |
| (SEQ ID NO: 97) | |
| | CᵢNLQAPCᵢᵢMLTGK[EPA]Cᵢᵢᵢ; |
| (SEQ ID NO: 98) | |
| | CᵢNLQQPCᵢᵢMLTGKVCᵢᵢᵢ; |
| (SEQ ID NO: 99) | |
| | CᵢNLQA[Nva]CᵢᵢMLTGKVCᵢᵢᵢ; |
| (SEQ ID NO: 100) | |
| | CᵢNLQAPCᵢᵢMHTGKVCᵢᵢᵢ; |
| (SEQ ID NO: 101) | |
| | CᵢNLQAPCᵢᵢLHTGKVCᵢᵢᵢ; |
| | and |
| (SEQ ID NO: 102) | |
| | CᵢNLQQACᵢᵢMQTGKVCᵢᵢᵢ; |

wherein Cᵢ, Cᵢᵢ and Cᵢᵢᵢ represent first, second and third cysteine residues, respectively, and wherein Cba represents cyclobutylalanine, tBuAla represents β-t-Butyl-L-alanine, Sar represents sarcosine, Cis-HyP represents L-4-cis-hydroxyproline, HyP represents hydroxyproline, NMeAla represents N-methylalanine, Ne represents norleucine, dA represents D-alanine, Agb represents norarginine, tBuGly represents tert-butylglycine, Cha represents 3-cyclohexyl-L-alanine, Chg represents cyclohexylglycine, Cbg represents cyclobutylglycine, HArg represents homoarginie, Aze represents azetidine-2-carboxylic acid, Pip represents pipecolic acid, Nva represents norvaline, Allolle represents L-allo-isoleucine, and EPA represents diethyl-L-alanine, or a pharmaceutically acceptable salt thereof.

In a yet further embodiment, said loop sequences comprise three cysteine residues separated by two loop sequences the first of which consists of 5 amino acids and the second of which consists of 6 amino acids and the bicyclic peptide ligand comprises an amino acid sequence which is selected from:

| (SEQ ID NO: 3) | |
|---|---|
| | CᵢNLQAPCᵢᵢMQTGKVCᵢᵢᵢ; |
| | and |
| (SEQ ID NO: 4) | |
| | CᵢNLQAPCᵢᵢMLTGKVCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 5 amino acids and the second of which consists of 6 amino acids, the molecular scaffold is TATA and the bicyclic peptide ligand additionally comprises N- and/or C-terminal additions and comprises an amino acid sequence which is selected from:
A-(SEQ ID NO: 3)-A-[K(PYA)] (herein referred to as BCY15687);
A-(SEQ ID NO: 3)-A (herein referred to as BCY15098);
A-(SEQ ID NO: 4)-A-Sar₆-KFI (herein referred to as BCY15115);
A-(SEQ ID NO: 4)-A (herein referred to as BCY15099);
Ac-(SEQ ID NO: 4) (herein referred to as BCY18293);
Ac-A-(SEQ ID NO: 4)-A (herein referred to as BCY18294);
(SEQ ID NO: 4)-A (herein referred to as BCY19694);
K-(SEQ ID NO: 4)-A (herein referred to as BCY19695);
dK-(SEQ ID NO: 4)-A (herein referred to as BCY19696);
Dap-(SEQ ID NO: 4)-A (herein referred to as BCY19697);
Dab-(SEQ ID NO: 4)-A (herein referred to as BCY19698);
GABA-(SEQ ID NO: 4)-A (herein referred to as BCY19699);
A-(SEQ ID NO: 63)-A (herein referred to as BCY14454);
A-(SEQ ID NO: 64)-A (herein referred to as BCY18299);
A-(SEQ ID NO: 65)-A (herein referred to as BCY18301);
A-(SEQ ID NO: 66)-A (herein referred to as BCY18302);
A-(SEQ ID NO: 67)-A (herein referred to as BCY18303);
A-(SEQ ID NO: 68)-A (herein referred to as BCY18304);
A-(SEQ ID NO: 69)-A (herein referred to as BCY18305);
A-(SEQ ID NO: 70)-A (herein referred to as BCY18307);
A-(SEQ ID NO: 71)-A (herein referred to as BCY18309);
A-(SEQ ID NO: 72)-A (herein referred to as BCY18310);
A-(SEQ ID NO: 73)-A (herein referred to as BCY18311);
A-(SEQ ID NO: 74)-A (herein referred to as BCY18312);
A-(SEQ ID NO: 75)-A (herein referred to as BCY18313);
A-(SEQ ID NO: 76)-A (herein referred to as BCY18314);
A-(SEQ ID NO: 77)-A (herein referred to as BCY18315);
A-(SEQ ID NO: 78)-A (herein referred to as BCY18316);
A-(SEQ ID NO: 79)-A (herein referred to as BCY18317);
A-(SEQ ID NO: 80)-A (herein referred to as BCY18318);
A-(SEQ ID NO: 81)-A (herein referred to as BCY18319);
A-(SEQ ID NO: 82)-A (herein referred to as BCY18320);
A-(SEQ ID NO: 83)-A (herein referred to as BCY18321);
A-(SEQ ID NO: 84)-A (herein referred to as BCY18322);
A-(SEQ ID NO: 85)-A (herein referred to as BCY18323);
A-(SEQ ID NO: 86)-A (herein referred to as BCY18324);
A-(SEQ ID NO: 87)-A (herein referred to as BCY18325);
A-(SEQ ID NO: 88)-A (herein referred to as BCY18326);
A-(SEQ ID NO: 89)-A (herein referred to as BCY18327);
A-(SEQ ID NO: 90)-A (herein referred to as BCY18328);
A-(SEQ ID NO: 91)-A (herein referred to as BCY19700);
A-(SEQ ID NO: 92)-A (herein referred to as BCY19701);
A-(SEQ ID NO: 93)-A (herein referred to as BCY19702);
A-(SEQ ID NO: 94)-A (herein referred to as BCY19703);
A-(SEQ ID NO: 95)-A (herein referred to as BCY19704);
A-(SEQ ID NO: 96)-A (herein referred to as BCY19706);
A-(SEQ ID NO: 97)-A (herein referred to as BCY19707);
A-(SEQ ID NO: 98)-A (herein referred to as BCY19708);
A-(SEQ ID NO: 99)-A (herein referred to as BCY19710);
A-(SEQ ID NO: 100)-A (herein referred to as BCY19711);
A-(SEQ ID NO: 101)-A (herein referred to as BCY19712); and
A-(SEQ ID NO: 102)-A (herein referred to as BCY19713),

wherein PYA represents pentynoic acid, Sar represents sarcosine, FI represents fluorescein, dK represents D-lysine, Dap represents L-2,3-diaminopropionic acid, Dab represents L-2,4-diaminobutyric acid, and GABA represents γ-aminobutyric acid.

In a still yet further embodiment, said loop sequences comprise three reactive groups separated by two loop sequences the first of which consists of 5 amino acids and the second of which consists of 6 amino acids, the molecular scaffold is TATA and the bicyclic peptide ligand additionally comprises N- and/or C-terminal additions and comprises an amino acid sequence which is selected from:
A-(SEQ ID NO: 3)-A-[K(PYA)] (herein referred to as BCY15687); and
A-(SEQ ID NO: 4)-A-Sar₆-KFI (herein referred to as BCY15115),

wherein PYA represents pentynoic acid, Sar represents sarcosine and FI represents fluorescein.

In an alternative embodiment, said loop sequences comprise three cysteine residues separated by two loop sequences the first of which consists of 5 amino acids and the second of which consists of 7 amino acids.

In a further embodiment, said loop sequences comprise three cysteine residues separated by two loop sequences the first of which consists of 5 amino acids and the second of which consists of 7 amino acids and the bicyclic peptide ligand comprises an amino acid sequence which is selected from:

| (SEQ ID NO: 5) | |
|---|---|
| | CᵢLLHDHCᵢᵢPNTHPKLCᵢᵢᵢ; |
| (SEQ ID NO: 6) | |
| | CᵢLLHEHCᵢᵢPNTHPKMCᵢᵢᵢ; |
| (SEQ ID NO: 7) | |
| | CᵢLLHDHCᵢᵢPNSHPEICᵢᵢᵢ; |
| (SEQ ID NO: 8) | |
| | CᵢLLHDHCᵢᵢPNTHPIICᵢᵢᵢ; |
| | and |
| (SEQ ID NO: 103) | |
| | CᵢLLHDHCᵢᵢPNTHPKMCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three cysteine residues separated by two loop sequences the first of which consists of 5 amino acids and the second of which consists of 7 amino acids and the bicyclic peptide ligand comprises an amino acid sequence which is selected from:

| (SEQ ID NO: 5) | |
|---|---|
| | CᵢLLHDHCᵢᵢPNTHPKLCᵢᵢᵢ; |
| (SEQ ID NO: 6) | |
| | CᵢLLHEHCᵢᵢPNTHPKMCᵢᵢᵢ; |
| (SEQ ID NO: 7) | |
| | CᵢLLHDHCᵢᵢPNSHPEICᵢᵢᵢ; |
| | and |
| (SEQ ID NO: 8) | |
| | CᵢLLHDHCᵢᵢPNTHPIICᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 5 amino acids and the second of which consists of 7 amino acids, the molecular scaffold is TATA and the bicyclic peptide ligand additionally comprises N- and/or C-terminal additions and comprises an amino acid sequence which is selected from:
A-(SEQ ID NO: 5)-A (herein referred to as BCY15103);
A-(SEQ ID NO: 5)-A-[K(PYA)] (herein referred to as BCY18005);
A-(SEQ ID NO: 5)-A-Sar₆-KFI (herein referred to as BCY15118);
A-(SEQ ID NO: 6)-A (herein referred to as BCY15104);
A-(SEQ ID NO: 6)-A-Sar₆-KFI (herein referred to as BCY15119);
A-(SEQ ID NO: 7)-A (herein referred to as BCY15105);
A-(SEQ ID NO: 7)-A-Sar₆-KFI (herein referred to as BCY15120);
A-(SEQ ID NO: 8)-A (herein referred to as BCY15106);
A-(SEQ ID NO: 8)-A-Sar₆-KFI (herein referred to as BCY15121); and
A-(SEQ ID NO: 103)-A (herein referred to as BCY14457),

wherein PYA represents pentynoic acid, Sar represents sarcosine and FI represents fluorescein.

In a still yet further embodiment, said loop sequences comprise three reactive groups separated by two loop sequences the first of which consists of 5 amino acids and the second of which consists of 7 amino acids, the molecular scaffold is TATA and the bicyclic peptide ligand additionally comprises N- and/or C-terminal additions and comprises an amino acid sequence which is selected from:
A-(SEQ ID NO: 5)-A-Sar₆-KFI (herein referred to as BCY15118);
A-(SEQ ID NO: 6)-A-Sar₆-KFI (herein referred to as BCY15119);
A-(SEQ ID NO: 7)-A-Sar₆-KFI (herein referred to as BCY15120); and
A-(SEQ ID NO: 8)-A-Sar₆-KFI (herein referred to as BCY15121),

wherein Sar represents sarcosine and FI represents fluorescein.

In an alternative embodiment, said loop sequences comprise three cysteine residues separated by two loop sequences the first of which consists of 6 amino acids and the second of which consists of 4 amino acids.

In a further embodiment, said loop sequences comprise three cysteine or penicillamine residues separated by two loop sequences the first of which consists of 6 amino acids and the second of which consists of 4 amino acids and the bicyclic peptide ligand comprises an amino acid sequence which is selected from:

| (SEQ ID NO: 9) | |
|---|---|
| | CᵢYLPDWLCᵢᵢGDEYCᵢᵢᵢ; |
| (SEQ ID NO: 10) | |
| | CᵢYLPDYLCᵢᵢGDEYCᵢᵢᵢ; |
| | (SEQ ID NO: 11; herein |
| | referred to as BCY18262 |
| | when complexed with TATA) |
| | CᵢDLTTHNCᵢᵢQWGICᵢᵢᵢ; |
| | (SEQ ID NO: 12; herein |
| | referred to as BCY15633 |
| | when complexed with TATA) |
| | CᵢYLPDYLCᵢᵢGDEYCᵢᵢᵢ; |
| (SEQ ID NO: 13) | |
| | CᵢYLPDWLCᵢᵢGDEYCᵢᵢᵢ; |
| (SEQ ID NO: 14) | |
| | Cᵢ[2,6DiMeTyr]LPDYLCᵢᵢGDEYCᵢᵢᵢ; |
| (SEQ ID NO: 15) | |
| | CᵢY[tBuAla]PDYLCᵢᵢGDEYCᵢᵢᵢ; |
| (SEQ ID NO: 16) | |
| | CᵢY[Cba]PDYLCᵢᵢGDEYCᵢᵢᵢ; |
| (SEQ ID NO: 17) | |
| | CᵢYL[HyP]DYLCᵢᵢGDEYCᵢᵢᵢ; |
| (SEQ ID NO: 18) | |
| | CᵢYL[Aze]DYLCᵢᵢGDEYCᵢᵢᵢ; |
| (SEQ ID NO: 19) | |
| | CᵢYL[Aib]DYLCᵢᵢGDEYCᵢᵢᵢ; |
| (SEQ ID NO: 20) | |
| | CYLPD[1Nal]LCᵢᵢGDEYCᵢᵢᵢ; |
| (SEQ ID NO: 21) | |
| | CᵢYLPD[2Nal]LCᵢᵢGDEYCᵢᵢᵢ; |
| (SEQ ID NO: 22) | |
| | CᵢYLPD[3Pal]LCᵢᵢGDEYCᵢᵢᵢ; |
| (SEQ ID NO: 23) | |
| | CᵢYLPD[2,6DiMeTyr]LCᵢᵢGDEYCᵢᵢᵢ; |
| (SEQ ID NO: 24) | |
| | CᵢYLPDW[tBuAla]CᵢᵢGDEYCᵢᵢᵢ; |
| (SEQ ID NO: 25) | |
| | CᵢYLPDW[Cba]CᵢᵢGDEYCᵢᵢᵢ; |
| (SEQ ID NO: 26) | |
| | CᵢYLPDYLCᵢᵢ[dA]DEYCᵢᵢᵢ; |
| (SEQ ID NO: 27) | |
| | CᵢYLPDYLCᵢᵢGDE[2,6DiMeTyr]Cᵢᵢᵢ; |
| (SEQ ID NO: 28) | |
| | CᵢALPDYLCᵢᵢGDEYCᵢᵢᵢ; |
| (SEQ ID NO: 29) | |
| | CᵢYAPDYLCᵢᵢGDEYCᵢᵢᵢ; |
| (SEQ ID NO: 30) | |
| | CᵢYLADYLCᵢᵢGDEYCᵢᵢᵢ; |
| (SEQ ID NO: 31) | |
| | CᵢYLPAYLCᵢᵢGDEYCᵢᵢᵢ; |
| (SEQ ID NO: 32) | |
| | CᵢYLPDALCᵢᵢGDEYCᵢᵢᵢ; |
| (SEQ ID NO: 33) | |
| | CᵢYLPDYACᵢᵢGDEYCᵢᵢᵢ; |
| (SEQ ID NO: 34) | |
| | CᵢYLPDYLCᵢᵢADEYCᵢᵢᵢ; |
| (SEQ ID NO: 35) | |
| | CᵢYLPDYLCᵢᵢGAEYCᵢᵢᵢ; |
| (SEQ ID NO: 36) | |
| | CᵢYLPDYLCᵢᵢGDAYCᵢᵢᵢ; |
| (SEQ ID NO: 37) | |
| | CᵢYLPDYLCᵢᵢGDEACᵢᵢᵢ; |
| (SEQ ID NO: 38) | |
| | CᵢY[C5A]PDYLCᵢᵢGDEYCᵢᵢᵢ; |
| (SEQ ID NO: 39) | |
| | CᵢY[Cha]PDYLCᵢᵢGDEYCᵢᵢᵢ; |
| (SEQ ID NO: 40) | |
| | CᵢY[Cba]PDYLCᵢᵢ[dA]DEYCᵢᵢᵢ; |
| (SEQ ID NO: 41) | |
| | CᵢYLPD[4FPhe]LCᵢᵢGDEYCᵢᵢᵢ; |
| (SEQ ID NO: 42) | |
| | CᵢYLPD[4MeOPhe]LCᵢᵢGDEYCᵢᵢᵢ; |
| (SEQ ID NO: 43) | |
| | CᵢYLPD[NO2Phe]LCᵢᵢGDEYCᵢᵢᵢ; |
| (SEQ ID NO: 44) | |
| | CᵢYLPD[4MePhe]LCᵢᵢGDEYCᵢᵢᵢ; |
| (SEQ ID NO: 45) | |
| | CᵢYLPD[pCoPhe]LCᵢᵢGDEYCᵢᵢᵢ; |
| (SEQ ID NO: 46) | |
| | CᵢYLPD[pCaPhe]LCᵢᵢGDEYCᵢᵢᵢ; |
| (SEQ ID NO: 47) | |
| | CᵢYLPD[4tBuPhe]LCᵢᵢGDEYCᵢᵢᵢ; |
| (SEQ ID NO: 48) | |
| | CᵢYLPD[CF3Phe]LCᵢᵢGDEYCᵢᵢᵢ; |
| (SEQ ID NO: 49) | |
| | CᵢYLPD[4CNPhe]LCᵢᵢGDEYCᵢᵢᵢ; |
| (SEQ ID NO: 50) | |
| | CᵢYLPD[4ClPhe]LCᵢᵢGDEYCᵢᵢᵢ; |
| (SEQ ID NO: 51) | |
| | CᵢYLPD[HPhe]LCᵢᵢGDEYCᵢᵢᵢ; |
| (SEQ ID NO: 52) | |
| | Cᵢ[2,6DiMeTyr]LPDWLCᵢᵢGDEYCᵢᵢᵢ; |
| (SEQ ID NO: 53) | |
| | CᵢY[Cba]PDWLCᵢᵢGDEYCᵢᵢᵢ; |
| (SEQ ID NO: 54) | |
| | CᵢYL[Aze]DWLCᵢᵢGDEYCᵢᵢᵢ; |
| (SEQ ID NO: 55) | |
| | CᵢYLPDWLCᵢᵢ[dA]DEYCᵢᵢᵢ; |
| (SEQ ID NO: 56) | |
| | CᵢSLPDYLCᵢᵢGDEYCᵢᵢᵢ; |
| (SEQ ID NO: 57) | |
| | CᵢDLTTHNCᵢᵢQWGICᵢᵢᵢ; |
| (SEQ ID NO: 58) | |
| | CᵢDLTTHNCᵢᵢLWGVCᵢᵢᵢ; |
| (SEQ ID NO: 59) | |
| | CᵢNLQLHNCᵢᵢIWGVCᵢᵢᵢ; |
| (SEQ ID NO: 104) | |
| | CᵢDLTTHNCᵢᵢIWGVCᵢᵢᵢ; |
| (SEQ ID NO: 105) | |
| | CᵢNLQLHNCᵢᵢIWGVCᵢᵢᵢ; |
| (SEQ ID NO: 106) | |
| | CᵢNLQLHNCᵢᵢQWGICᵢᵢᵢ; |
| (SEQ ID NO: 107) | |
| | Cᵢ[3tBuTyr]LPDYLCᵢᵢGDEYCᵢᵢᵢ; |
| (SEQ ID NO: 108) | |
| | CᵢYLPD[3tBuTyr]LCᵢᵢGDEYCᵢᵢᵢ; |
| (SEQ ID NO: 109) | |
| | CᵢYLPDYLCᵢᵢGDE[3tBuTyr]Cᵢᵢᵢ; |
| (SEQ ID NO: 110) | |
| | Cᵢ[K(PYA)]LPDYLCᵢᵢGDEYCᵢᵢᵢ; |
| (SEQ ID NO: 111) | |
| | CᵢYLP[K(PYA)]YLCᵢᵢGDEYCᵢᵢᵢ; |
| (SEQ ID NO: 112) | |
| | CᵢYLPDYLCᵢᵢ[K(PYA)]DEYCᵢᵢᵢ; |
| (SEQ ID NO: 113) | |
| | CᵢYLPDYLCᵢᵢGD[K(PYA)]YCᵢᵢᵢ; |
| (SEQ ID NO: 114) | |
| | CᵢYLPDYLCᵢᵢGDE[K(PYA)]Cᵢᵢᵢ; |
| (SEQ ID NO: 115) | |
| | CᵢYLPDYLCᵢᵢ[dK(PYA)]DEYCᵢᵢᵢ; |
| (SEQ ID NO: 116) | |
| | CᵢALTTHNCᵢᵢQWGICᵢᵢᵢ; |
| (SEQ ID NO: 117) | |
| | CᵢDLATHNCᵢᵢQWGICᵢᵢᵢ; |
| (SEQ ID NO: 118) | |
| | CᵢDLTAHNCᵢᵢQWGICᵢᵢᵢ; |
| (SEQ ID NO: 119) | |
| | CᵢDLTTHNCᵢᵢAWGICᵢᵢᵢ; |
| (SEQ ID NO: 120) | |
| | CᵢDLTTHNCᵢᵢQWGACᵢᵢᵢ; |
| (SEQ ID NO: 121) | |
| | CᵢNLTTHNCᵢᵢQWGICᵢᵢᵢ; |
| (SEQ ID NO: 122) | |
| | CᵢDLQTHNCᵢᵢQWGICᵢᵢᵢ; |
| (SEQ ID NO: 123) | |
| | CᵢDLTLHNCᵢᵢQWGICᵢᵢᵢ; |
| (SEQ ID NO: 124) | |
| | CᵢDLTT[His3Me]NCᵢᵢQWGICᵢᵢᵢ; |
| (SEQ ID NO: 125) | |
| | CᵢDLTT[2Pal]NCᵢᵢQWGICᵢᵢᵢ; |
| (SEQ ID NO: 126) | |
| | CᵢDLTT[His1Me]NCᵢᵢQWGICᵢᵢᵢ; |
| (SEQ ID NO: 127) | |
| | CᵢDLTT[4ThiAz]NCᵢᵢQWGICᵢᵢᵢ; |
| (SEQ ID NO: 128) | |
| | CᵢDLTTHNCᵢᵢVWGICᵢᵢᵢ; |
| (SEQ ID NO: 129) | |
| | CᵢDLTTHNCᵢᵢ[Cba]WGICᵢᵢᵢ; |
| (SEQ ID NO: 130) | |
| | CᵢDLTTHNCᵢᵢQ[1Nal]GICᵢᵢᵢ; |
| (SEQ ID NO: 131) | |
| | CᵢDLTTHNCᵢᵢQ[Trp(Me)]GICᵢᵢᵢ; |
| (SEQ ID NO: 132) | |
| | CᵢDLTTHNCᵢᵢQWG[BuGly]Cᵢᵢᵢ; |
| (SEQ ID NO: 133) | |
| | CᵢDLTTHNCᵢᵢQWG[tBuAla]Cᵢᵢᵢ; |
| (SEQ ID NO: 134) | |
| | CᵢDLTTHNCᵢᵢQWG[Chg]Cᵢᵢᵢ; |
| (SEQ ID NO: 135) | |
| | CᵢDLTTHNCᵢᵢQW[dA]ICᵢᵢᵢ; |
| (SEQ ID NO: 136) | |
| | CᵢS[Cba]PDYLCᵢᵢ[dA]DEYCᵢᵢᵢ; |
| (SEQ ID NO: 137) | |
| | Cᵢ[Hser][Cba]PDYLCᵢᵢ[dA]DEYCᵢᵢᵢ; |
| (SEQ ID NO: 138) | |
| | CᵢN[Cba]PDYLCᵢᵢ[dA]DEYCᵢᵢᵢ; |
| (SEQ ID NO: 139) | |
| | Cᵢ[4Pal][Cba]PDYLCᵢᵢ[dA]DEYCᵢᵢᵢ; |
| (SEQ ID NO: 140) | |
| | CᵢY[Cba][Cis-HyP]DYLCᵢᵢ[dA]DEYCᵢᵢᵢ; |
| (SEQ ID NO: 141) | |
| | CᵢY[Cba][NmeAla]DYLCᵢᵢ[dA]DEYCᵢᵢᵢ; |
| (SEQ ID NO: 142) | |
| | CᵢY[Cba]PNYLCᵢᵢ[dA]DEYCᵢᵢᵢ; |
| (SEQ ID NO: 143) | |
| | CᵢY[Cba]PQYLCᵢᵢ[dA]DEYCᵢᵢᵢ; |
| (SEQ ID NO: 144) | |
| | CᵢY[Cba]PDYLCᵢᵢ[dV]DEYCᵢᵢᵢ; |
| (SEQ ID NO: 145) | |
| | CᵢY[Cba]PDYLCᵢᵢ[dNva]DEYCᵢᵢᵢ; |
| (SEQ ID NO: 146) | |
| | CᵢY[Cba]PDYLCᵢᵢ[dF]DEYCᵢᵢᵢ; |
| (SEQ ID NO: 147) | |
| | CᵢY[Cba]PDYLCᵢᵢ[dCha]DEYCᵢᵢᵢ; |
| (SEQ ID NO: 148) | |
| | CᵢY[Cba]PDYLCᵢᵢ[dK(PYA)]DEYCᵢᵢᵢ; |
| (SEQ ID NO: 149) | |
| | CᵢY[Cba]PDYLCᵢᵢ[dNle]DEYCᵢᵢᵢ; |
| (SEQ ID NO: 150) | |
| | CᵢY[Cba]PD[DOPA]LCᵢᵢ[dA]DEYCᵢᵢᵢ; |
| (SEQ ID NO: 151) | |
| | CᵢY[Cba]PD[2FTyr]LCᵢᵢ[dA]DEYCᵢᵢᵢ; |
| (SEQ ID NO: 152) | |
| | CᵢY[Cba]PDY[Nle]Cᵢᵢ[dA]DEYCᵢᵢᵢ; |
| (SEQ ID NO: 153) | |
| | [Pen]ᵢY[Cba]PDYLCᵢᵢ[dA]DEYCᵢᵢᵢ; |
| (SEQ ID NO: 154) | |
| | CᵢY[Cba]PDYL[Pen]ᵢᵢ[dA]DEYCᵢᵢᵢ; |
| (SEQ ID NO: 155) | |
| | CᵢY[Cba]PDYLCᵢᵢ[dA]DEY[Pen]ᵢᵢᵢ; |
| (SEQ ID NO: 156) | |
| | CᵢY[Cba]PDYLCᵢᵢ[dA]DEY[dCᵢᵢᵢ]; |
| (SEQ ID NO: 157) | |
| | [dCᵢ]Y[Cba]PDYLCᵢᵢ[dA]DEYCᵢᵢᵢ; |
| (SEQ ID NO: 158) | |
| | CᵢDLTTHNCᵢᵢQ[AzaTrp]GICᵢᵢᵢ; |
| (SEQ ID NO: 159) | |
| | CᵢDLTTHNCᵢᵢQ[2MeTrp]GICᵢᵢᵢ; |
| (SEQ ID NO: 160) | |
| | CᵢDLTTHNCᵢᵢQ[6MeTrp]GICᵢᵢᵢ; |
| (SEQ ID NO: 161) | |
| | CᵢDLTTHNCᵢᵢQ[7MeTrp]GICᵢᵢᵢ; |
| (SEQ ID NO: 162) | |
| | CᵢDLTTHNCᵢᵢQ[6FTrp]GICᵢᵢᵢ; |
| (SEQ ID NO: 163) | |
| | CᵢDLTTHNCᵢᵢQ[5FTrp]GICᵢᵢᵢ; |
| (SEQ ID NO: 164) | |
| | CᵢDLTTHNCᵢᵢQ[Dht]GICᵢᵢᵢ; |
| (SEQ ID NO: 165) | |
| | CᵢDLTT[2FuAla]NCᵢᵢQWGICᵢᵢᵢ; |
| (SEQ ID NO: 166) | |
| | CᵢDLTTHNCᵢᵢQWG[EPA]Cᵢᵢᵢ; |
| (SEQ ID NO: 167) | |
| | CᵢDLTTHNCᵢᵢQWG[Nle]Cᵢᵢᵢ; |
| | and |
| (SEQ ID NO: 168) | |
| | CᵢDLTTHNCᵢᵢQWG[Allolle]Cᵢᵢᵢ; |

wherein Cᵢ, Penᵢ, Cᵢᵢ, Penᵢᵢ, Cᵢᵢᵢ and Penᵢᵢᵢ represent first, second and third reactive groups, respectively, wherein 2,6DiMeTyr represents 2,6-dimethyl-tyrosine, tBuAla represents t-butyl-alanine, Cba represents β-cyclobutylalanine, HyP represents hydroxyproline, Aze represents azetidine, Aib represents aminoisobutyric acid, 1NaI represents 1-naphthylalanine, 2NaI represents 2-naphthylalanine, 3Pal represents 3-pyridylalanine, C5A represents beta-cyclopentyl-L-alanine, Cha represents 3-cyclohexyl-L-alanine, 4Fphe represents 4-fluoro-L-phenylalanine, 4MeOPhe represents 4-methoxy-phenylalanine, NO2Phe represents 4-nitro-phenylalanine, 4MePhe represents 4-methyl-phenylalanine, pCoPhe represents para-carboxy-phenylalanine, pCaPhe represents β-carboxamido-phenylalanine, 4tBuPhe represents 4-tert-butyl-phenylalanine, CF3Phe represents 4-trifluoromethyl-phenylalanine, 4CNPhe represents 4-cyano-phenylalanine, 4CIPhe represents 4-chloro-phenylalanine, HPhe represents homophenylalanine, 3tBuTyr represents 3-tert-butyltyrosine, His3Me represents 3-methylhistidine, 2Pal represents 2-pyridylalanine, His1Me represents 1-methylhistidine, 4ThiAz represents beta-4-thiazol-L-ylalanine, Trp(Me) represents 1-methyltryptophan, tBuGly represents L-t-butylglycine, Chg represents cyclohexylglycine, Hser represents homoserine, NmeAla represents N-methylalanine, 4Pal represents 4-pyridylalanine, Cis-HyP represents 4-cis-hydroxyproline, dA represents D-Alanine, dV represents D-valine, dNva represents D-norvaline, dNle represents D-norleucine, dF represents D-phenylalanine, dCha represents 3-cyclohexyl-D-alanine, DOPA represents 3,4-Dihydroxyphenylalanine, 2FTyr represents 2-fluorotyrosine, Ne represents norleucine, dC represents D-cysteine, AzaTrp represents azatryptophan, 2MeTrp represents 2-methyltryptophan, 6MeTrp represents 6-methyltryptophan, 6FTrp represents 6-fluorotryptophan, 5FTrp represents 5-fluorotryptophan, Dht represents 2,3-Dihydrotryptophan, 2FuAla represents 2-furylalanine, Allolle represents L-allo-isoleucine or a pharmaceutically acceptable salt thereof.

In a yet further embodiment, said loop sequences comprise three cysteine residues separated by two loop sequences the first of which consists of 6 amino acids and the second of which consists of 4 amino acids and the bicyclic peptide ligand comprises an amino acid sequence which is selected from:

| (SEQ ID NO: 9) | |
|---|---|
| | CᵢYLPDWLCᵢᵢGDEYCᵢᵢᵢ; |
| (SEQ ID NO: 10) | |
| | CᵢYLPDYLCᵢᵢGDEYCᵢᵢᵢ; |
| (SEQ ID NO: 11) | |
| | CᵢDLTTHNCᵢᵢQWGICᵢᵢᵢ; |
| | (SEQ ID NO: 12; herein |
| | referred to as BCY15633 |
| | when complexed with TATA) |
| | CᵢYLPDYLCᵢᵢGDEYCᵢᵢᵢ; |
| (SEQ ID NO: 13) | |
| | CᵢYLPDWLCᵢᵢGDEYCᵢᵢᵢ; |
| (SEQ ID NO: 14) | |
| | Cᵢ[2,6DiMeTyr]LPDYLCᵢᵢGDEYCᵢᵢᵢ; |
| (SEQ ID NO: 15) | |
| | CᵢY[tBuAla]PDYLCᵢᵢGDEYCᵢᵢᵢ; |
| (SEQ ID NO: 16) | |
| | CᵢY[Cba]PDYLCᵢᵢGDEYCᵢᵢᵢ; |
| (SEQ ID NO: 17) | |
| | CᵢYL[HyP]DYLCᵢᵢGDEYCᵢᵢᵢ; |
| (SEQ ID NO: 18) | |
| | CᵢYL[Aze]DYLCᵢᵢGDEYCᵢᵢᵢ; |
| (SEQ ID NO: 19) | |
| | CᵢYL[Aib]DYLCᵢᵢGDEYCᵢᵢᵢ; |
| (SEQ ID NO: 20) | |
| | CᵢYLPD[1Nal]LCᵢᵢGDEYCᵢᵢᵢ; |
| (SEQ ID NO: 21) | |
| | CᵢYLPD[2Nal]LCᵢᵢGDEYCᵢᵢᵢ; |
| (SEQ ID NO: 22) | |
| | CᵢYLPD[3Pal]LCᵢᵢGDEYCᵢᵢᵢ; |
| (SEQ ID NO: 23) | |
| | CᵢYLPD[2,6DiMeTyr]LCᵢᵢGDEYCᵢᵢᵢ; |
| (SEQ ID NO: 24) | |
| | CᵢYLPDW[tBuAla]CᵢᵢGDEYCᵢᵢᵢ; |
| (SEQ ID NO: 25) | |
| | CᵢYLPDW[Cba]CᵢᵢGDEYCᵢᵢᵢ; |
| (SEQ ID NO: 26) | |
| | CᵢYLPDYLCᵢᵢ[dA]DEYCᵢᵢᵢ; |
| (SEQ ID NO: 27) | |
| | CᵢYLPDYLCᵢᵢGDE[2,6DiMeTyr]Cᵢᵢᵢ; |
| (SEQ ID NO: 28) | |
| | CᵢALPDYLCᵢᵢGDEYCᵢᵢᵢ; |
| (SEQ ID NO: 29) | |
| | CᵢYAPDYLCᵢᵢGDEYCᵢᵢᵢ; |
| (SEQ ID NO: 30) | |
| | CᵢYLADYLCᵢᵢGDEYCᵢᵢᵢ; |
| (SEQ ID NO: 31) | |
| | CᵢYLPAYLCᵢᵢGDEYCᵢᵢᵢ; |
| (SEQ ID NO: 32) | |
| | CᵢYLPDALCᵢᵢGDEYCᵢᵢᵢ; |
| (SEQ ID NO: 33) | |
| | CᵢYLPDYACᵢᵢGDEYCᵢᵢᵢ; |
| (SEQ ID NO: 34) | |
| | CᵢYLPDYLCᵢᵢADEYCᵢᵢᵢ; |
| (SEQ ID NO: 35) | |
| | CᵢYLPDYLCᵢᵢGAEYCᵢᵢᵢ; |
| (SEQ ID NO: 36) | |
| | CᵢYLPDYLCᵢᵢGDAYCᵢᵢᵢ; |
| (SEQ ID NO: 37) | |
| | CᵢYLPDYLCᵢᵢGDEACᵢᵢᵢ; |
| (SEQ ID NO: 38) | |
| | CᵢY[C5A]PDYLCᵢᵢGDEYCᵢᵢᵢ; |
| (SEQ ID NO: 39) | |
| | CᵢY[Cha]PDYLCᵢᵢGDEYCᵢᵢᵢ; |
| (SEQ ID NO: 40) | |
| | CᵢY[Cba]PDYLCᵢᵢ[dA]DEYCᵢᵢᵢ; |
| (SEQ ID NO: 41) | |
| | CᵢYLPD[4Fphe]LCᵢᵢGDEYCᵢᵢᵢ; |
| (SEQ ID NO: 42) | |
| | CᵢYLPD[4MeOPhe]LCᵢᵢGDEYCᵢᵢᵢ; |
| (SEQ ID NO: 43) | |
| | CᵢYLPD[NO2Phe]LCᵢᵢGDEYCᵢᵢᵢ; |
| (SEQ ID NO: 44) | |
| | CᵢYLPD[4MePhe]LCᵢᵢGDEYCᵢᵢᵢ; |
| (SEQ ID NO: 45) | |
| | CᵢYLPD[pCoPhe]LCᵢᵢGDEYCᵢᵢᵢ; |
| (SEQ ID NO: 46) | |
| | CᵢYLPD[pCaPhe]LCᵢᵢGDEYCᵢᵢᵢ; |
| (SEQ ID NO: 47) | |
| | CᵢYLPD[4tBuPhe]LCᵢᵢGDEYCᵢᵢᵢ; |
| (SEQ ID NO: 48) | |
| | CᵢYLPD[CF3Phe]LCᵢᵢGDEYCᵢᵢᵢ; |
| (SEQ ID NO: 49) | |
| | CᵢYLPD[4CNPhe]LCᵢᵢGDEYCᵢᵢᵢ; |
| (SEQ ID NO: 50) | |
| | CᵢYLPD[4ClPhe]LCᵢᵢGDEYCᵢᵢᵢ; |
| (SEQ ID NO: 51) | |
| | CᵢYLPD[HPhe]LCᵢᵢGDEYCᵢᵢᵢ; |
| (SEQ ID NO: 52) | |
| | Cᵢ[2,6DiMeTyr]LPDWLCᵢᵢGDEYCᵢᵢᵢ; |
| (SEQ ID NO: 53) | |
| | CᵢY[Cba]PDWLCᵢᵢGDEYCᵢᵢᵢ; |
| (SEQ ID NO: 54) | |
| | CᵢYL[Aze]DWLCᵢᵢGDEYCᵢᵢᵢ; |
| (SEQ ID NO: 55) | |
| | CᵢYLPDWLCᵢᵢ[dA]DEYCᵢᵢᵢ; |
| (SEQ ID NO: 56) | |
| | CᵢSLPDYLCᵢᵢGDEYCᵢᵢᵢ; |
| (SEQ ID NO: 57) | |
| | CᵢDLTTHNCᵢᵢQWGICᵢᵢᵢ; |
| (SEQ ID NO: 58) | |
| | CᵢDLTTHNCᵢᵢLWGVCᵢᵢᵢ; |
| | and |
| (SEQ ID NO: 59) | |
| | CᵢNLQLHNCHIWGVCᵢᵢᵢ; |

wherein Cᵢ, Cᵢᵢ and Cᵢᵢᵢ represent first, second and third cysteine residues, respectively, wherein 2,6DiMeTyr represents 2,6-dimethyl-tyrosine, tBuAla represents t-butyl-alanine, Cba represents β-cyclobutylalanine, HyP represents hydroxyproline, Aze represents azetidine, Aib represents aminoisobutyric acid, 1NaI represents 1-naphthylalanine, 2NaI represents 2-naphthylalanine, 3Pal represents 3-pyridylalanine, C5A represents beta-cyclopentyl-L-alanine, Cha represents 3-cyclohexyl-L-alanine, 4Fphe represents 4-fluoro-L-phenylalanine, 4MeOPhe represents 4-methoxy-phenylalanine, NO2Phe represents 4-nitro-phenylalanine, 4MePhe represents 4-methyl-phenylalanine, pCoPhe represents para-carboxy-phenylalanine, pCaPhe represents β-carboxamido-phenylalanine, 4tBuPhe represents 4-tert-butyl-phenylalanine, CF3Phe represents 4-trifluoromethyl-phenylalanine, 4CNPhe represents 4-cyano-phenylalanine, 4CIPhe represents 4-chloro-phenylalanine, HPhe represents homophenylalanine, or a pharmaceutically aceptable salt thereof.

In a yet further embodiment, said loop sequences comprise three reactive groups separated by two loop sequences the first of which consists of 6 amino acids and the second of which consists of 4 amino acids, the molecular scaffold is TATA and the bicyclic peptide ligand additionally comprises N- and/or C-terminal additions and comprises an amino acid sequence which is selected from:
A-(SEQ ID NO: 9)-A-[dK(PYA)] (herein referred to as BCY15013);
A-(SEQ ID NO: 10)-A (herein referred to as BCY18470);
A-(SEQ ID NO: 10)-A-[dK(PYA)] (herein referred to as BCY15452);
A-(SEQ ID NO: 11)-A (herein referred to as BCY15107); Ac-(SEQ ID NO: 11) (herein referred to as BCY18260);
Ac-A-(SEQ ID NO: 11)-A (herein referred to as BCY18261);
A-(SEQ ID NO: 11)-[Aib] (herein referred to as BCY19717);
(SEQ ID NO: 11)-A (herein referred to as BCY19718);
A-(SEQ ID NO: 11)-A-[dK(PYA)] (herein referred to as BCY19719);
A-(SEQ ID NO: 11)-A-[K(PYA)] (herein referred to as BCY15686);
A-(SEQ ID NO: 12)-A (herein referred to as BCY15100);
Ac-(SEQ ID NO: 12) (herein referred to as BCY15634);
Ac-A-(SEQ ID NO: 12)-A (herein referred to as BCY15635);
dA-(SEQ ID NO: 12) (herein referred to as BCY16134;
A-(SEQ ID NO: 12)-A-Sar₆-KFI (herein referred to as BCY15116);
A-(SEQ ID NO: 13)-A (herein referred to as BCY14455);
(SEQ ID NO: 13)-Sar₆-KFI (herein referred to as BCY15023);
Ac-(SEQ ID NO: 13)-Sar₆-KFI (herein referred to as BCY15024);
A-(SEQ ID NO: 13)-A-Sar₆-KFI (herein referred to as BCY14652);
A-(SEQ ID NO: 14)-A (herein referred to as BCY15636);
A-(SEQ ID NO: 15)-A (herein referred to as BCY15638);
A-(SEQ ID NO: 16)-A (herein referred to as BCY15639);
A-(SEQ ID NO: 17)-A (herein referred to as BCY15640);
A-(SEQ ID NO: 18)-A (herein referred to as BCY15641);
A-(SEQ ID NO: 19)-A (herein referred to as BCY15642);
A-(SEQ ID NO: 20)-A (herein referred to as BCY15643);
A-(SEQ ID NO: 21)-A (herein referred to as BCY15644);
A-(SEQ ID NO: 22)-A (herein referred to as BCY15645);
A-(SEQ ID NO: 23)-A (herein referred to as BCY15646);
A-(SEQ ID NO: 24)-A (herein referred to as BCY15648);
A-(SEQ ID NO: 25)-A (herein referred to as BCY15649);
A-(SEQ ID NO: 26)-A (herein referred to as BCY15650);
A-(SEQ ID NO: 27)-A (herein referred to as BCY15651);
A-(SEQ ID NO: 28)-A (herein referred to as BCY15653);
A-(SEQ ID NO: 29)-A (herein referred to as BCY15654);
A-(SEQ ID NO: 30)-A (herein referred to as BCY15655);
A-(SEQ ID NO: 31)-A (herein referred to as BCY15656);
A-(SEQ ID NO: 32)-A (herein referred to as BCY15657);
A-(SEQ ID NO: 33)-A (herein referred to as BCY15658);
A-(SEQ ID NO: 34)-A (herein referred to as BCY15659);
A-(SEQ ID NO: 35)-A (herein referred to as BCY15660);
A-(SEQ ID NO: 36)-A (herein referred to as BCY15661);
A-(SEQ ID NO: 37)-A (herein referred to as BCY15662);
A-(SEQ ID NO: 38)-A (herein referred to as BCY16130);
A-(SEQ ID NO: 39)-A (herein referred to as BCY16131);
A-(SEQ ID NO: 40)-A (herein referred to as BCY16132);
Ac-(SEQ ID NO: 40) (herein referred to as BCY16133);
Ac-(SEQ ID NO: 40)-[K(PYA)] (herein referred to as BCY17224);
A-(SEQ ID NO: 41)-A (herein referred to as BCY16135);
A-(SEQ ID NO: 42)-A (herein referred to as BCY16136);
A-(SEQ ID NO: 43)-A (herein referred to as BCY16137);
A-(SEQ ID NO: 44)-A (herein referred to as BCY16138);
A-(SEQ ID NO: 45)-A (herein referred to as BCY16139);
A-(SEQ ID NO: 46)-A (herein referred to as BCY16140);
A-(SEQ ID NO: 47)-A (herein referred to as BCY16141);
A-(SEQ ID NO: 48)-A (herein referred to as BCY16142);
A-(SEQ ID NO: 49)-A (herein referred to as BCY16143);
A-(SEQ ID NO: 50)-A (herein referred to as BCY16144);
A-(SEQ ID NO: 51)-A (herein referred to as BCY16145);
A-(SEQ ID NO: 52)-A-Sar₆-KFI (herein referred to as BCY15025);
A-(SEQ ID NO: 53)-A-Sar₆-KFI (herein referred to as BCY15028);
A-(SEQ ID NO: 54)-A-Sar₆-KFI (herein referred to as BCY15030);
A-(SEQ ID NO: 55)-A-Sar₆-KFI (herein referred to as BCY15035);
A-(SEQ ID NO: 56)-A-Sar₆-KFI (herein referred to as BCY15117);
A-(SEQ ID NO: 57)-A-Sar₆-KFI (herein referred to as BCY15122);
A-(SEQ ID NO: 58)-A (herein referred to as BCY15108);
A-(SEQ ID NO: 58)-A-Sar₆-KFI (herein referred to as BCY15123);
A-(SEQ ID NO: 59)-A-Sar₆-KFI (herein referred to as BCY15124),
A-(SEQ ID NO: 104)-A (herein referred to as BCY14458);
A-(SEQ ID NO: 105)-A (herein referred to as BCY15109);
A-(SEQ ID NO: 106)-A (herein referred to as BCY15568);
A-(SEQ ID NO: 107)-A (herein referred to as BCY15637);
A-(SEQ ID NO: 108)-A (herein referred to as BCY15647);
A-(SEQ ID NO: 109)-A (herein referred to as BCY15652);
A-(SEQ ID NO: 110)-A (herein referred to as BCY16834);
A-(SEQ ID NO: 111)-A (herein referred to as BCY16837);
A-(SEQ ID NO: 112)-A (herein referred to as BCY16840);
A-(SEQ ID NO: 113)-A (herein referred to as BCY16842);
A-(SEQ ID NO: 114)-A (herein referred to as BCY16843);
A-(SEQ ID NO: 115)-A (herein referred to as BCY17662);
A-(SEQ ID NO: 116)-A (herein referred to as BCY18263);
A-(SEQ ID NO: 117)-A (herein referred to as BCY18265);
A-(SEQ ID NO: 118)-A (herein referred to as BCY18266);
A-(SEQ ID NO: 119)-A (herein referred to as BCY18269);
A-(SEQ ID NO: 120)-A (herein referred to as BCY18272);
A-(SEQ ID NO: 121)-A (herein referred to as BCY18273);
A-(SEQ ID NO: 122)-A (herein referred to as BCY18277);
A-(SEQ ID NO: 123)-A (herein referred to as BCY18278);
A-(SEQ ID NO: 124)-A (herein referred to as BCY18279);
A-(SEQ ID NO: 125)-A (herein referred to as BCY18280);
A-(SEQ ID NO: 126)-A (herein referred to as BCY18281);
A-(SEQ ID NO: 127)-A (herein referred to as BCY18282);
A-(SEQ ID NO: 128)-A (herein referred to as BCY18285);
A-(SEQ ID NO: 129)-A (herein referred to as BCY18286);
A-(SEQ ID NO: 130)-A (herein referred to as BCY18287);
A-(SEQ ID NO: 131)-A (herein referred to as BCY18288);
A-(SEQ ID NO: 132)-A (herein referred to as BCY18289);
A-(SEQ ID NO: 133)-A (herein referred to as BCY18290);
A-(SEQ ID NO: 134)-A (herein referred to as BCY18291);
A-(SEQ ID NO: 135)-A (herein referred to as BCY18292);
Ac-(SEQ ID NO: 136) (herein referred to as BCY18433);
Ac-(SEQ ID NO: 137) (herein referred to as BCY18434);
Ac-(SEQ ID NO: 138) (herein referred to as BCY18435);
Ac-(SEQ ID NO: 139) (herein referred to as BCY18437);
Ac-(SEQ ID NO: 140) (herein referred to as BCY18439);
Ac-(SEQ ID NO: 141) (herein referred to as BCY18440);
Ac-(SEQ ID NO: 142) (herein referred to as BCY18441);
Ac-(SEQ ID NO: 143) (herein referred to as BCY18442);
Ac-(SEQ ID NO: 144) (herein referred to as BCY18443);
Ac-(SEQ ID NO: 145) (herein referred to as BCY18444);
Ac-(SEQ ID NO: 146) (herein referred to as BCY19682);
Ac-(SEQ ID NO: 147) (herein referred to as BCY19683);
Ac-(SEQ ID NO: 148) (herein referred to as BCY19684);
Ac-(SEQ ID NO: 149) (herein referred to as BCY19685);
Ac-(SEQ ID NO: 150) (herein referred to as BCY19686);
Ac-(SEQ ID NO: 151) (herein referred to as BCY19687);
Ac-(SEQ ID NO: 152) (herein referred to as BCY19688);
Ac-(SEQ ID NO: 153) (herein referred to as BCY19689);
Ac-(SEQ ID NO: 154) (herein referred to as BCY19690);
Ac-(SEQ ID NO: 155) (herein referred to as BCY19691);
Ac-(SEQ ID NO: 156) (herein referred to as BCY19692);
Ac-(SEQ ID NO: 157) (herein referred to as BCY19693);
A-(SEQ ID NO: 158)-A (herein referred to as BCY19720);
A-(SEQ ID NO: 159)-A (herein referred to as BCY19721);
A-(SEQ ID NO: 160)-A (herein referred to as BCY19722);
A-(SEQ ID NO: 161)-A (herein referred to as BCY19723);
A-(SEQ ID NO: 162)-A (herein referred to as BCY19724);
A-(SEQ ID NO: 163)-A (herein referred to as BCY19725);
A-(SEQ ID NO: 164)-A (herein referred to as BCY19730);
A-(SEQ ID NO: 165)-A (herein referred to as BCY19732);
A-(SEQ ID NO: 166)-A (herein referred to as BCY19733);
A-(SEQ ID NO: 167)-A (herein referred to as BCY19734); and
A-(SEQ ID NO: 168)-A (herein referred to as BCY19735);

wherein Aib represents aminoisobutyric acid, PYA represents pentynoic acid, Sar represents sarcosine and FI represents fluorescein.

In a still yet further embodiment, said loop sequences comprise three reactive groups separated by two loop sequences the first of which consists of 6 amino acids and the second of which consists of 4 amino acids, the molecular scaffold is TATA and the bicyclic peptide ligand additionally comprises N- and/or C-terminal additions and comprises an amino acid sequence which is selected from:
A-(SEQ ID NO: 9)-A-[dK(PYA)] (herein referred to as BCY15013);
A-(SEQ ID NO: 10)-A-[dK(PYA)] (herein referred to as BCY15452);
A-(SEQ ID NO: 11)-A-[K(PYA)] (herein referred to as BCY15686);
A-(SEQ ID NO: 12)-A (herein referred to as BCY15100);
(SEQ ID NO: 12) (herein referred to as BCY15633);
Ac-(SEQ ID NO: 12) (herein referred to as BCY15634);
Ac-A-(SEQ ID NO: 12)-A (herein referred to as BCY15635);
dA-(SEQ ID NO: 12) (herein referred to as BCY16134;
A-(SEQ ID NO: 12)-A-Sar₆-KFI (herein referred to as BCY15116);
A-(SEQ ID NO: 13)-A (herein referred to as BCY14455);
(SEQ ID NO: 13)-Sar₆-KFI (herein referred to as BCY15023);
Ac-(SEQ ID NO: 13)-Sar₆-KFI (herein referred to as BCY15024);
A-(SEQ ID NO: 13)-A-Sar₆-KFI (herein referred to as BCY14652);
A-(SEQ ID NO: 14)-A (herein referred to as BCY15636);
A-(SEQ ID NO: 15)-A (herein referred to as BCY15638);
A-(SEQ ID NO: 16)-A (herein referred to as BCY15639);
A-(SEQ ID NO: 17)-A (herein referred to as BCY15640);
A-(SEQ ID NO: 18)-A (herein referred to as BCY15641);
A-(SEQ ID NO: 19)-A (herein referred to as BCY15642);
A-(SEQ ID NO: 20)-A (herein referred to as BCY15643);
A-(SEQ ID NO: 21)-A (herein referred to as BCY15644);
A-(SEQ ID NO: 22)-A (herein referred to as BCY15645);
A-(SEQ ID NO: 23)-A (herein referred to as BCY15646);
A-(SEQ ID NO: 24)-A (herein referred to as BCY15648);
A-(SEQ ID NO: 25)-A (herein referred to as BCY15649);
A-(SEQ ID NO: 26)-A (herein referred to as BCY15650);
A-(SEQ ID NO: 27)-A (herein referred to as BCY15651);
A-(SEQ ID NO: 28)-A (herein referred to as BCY15653);
A-(SEQ ID NO: 29)-A (herein referred to as BCY15654);
A-(SEQ ID NO: 30)-A (herein referred to as BCY15655);
A-(SEQ ID NO: 31)-A (herein referred to as BCY15656);
A-(SEQ ID NO: 32)-A (herein referred to as BCY15657);
A-(SEQ ID NO: 33)-A (herein referred to as BCY15658);
A-(SEQ ID NO: 34)-A (herein referred to as BCY15659);
A-(SEQ ID NO: 35)-A (herein referred to as BCY15660);
A-(SEQ ID NO: 36)-A (herein referred to as BCY15661);
A-(SEQ ID NO: 37)-A (herein referred to as BCY15662);
A-(SEQ ID NO: 38)-A (herein referred to as BCY16130);
A-(SEQ ID NO: 39)-A (herein referred to as BCY16131);
A-(SEQ ID NO: 40)-A (herein referred to as BCY16132);
Ac-(SEQ ID NO: 40) (herein referred to as BCY16133);
Ac-(SEQ ID NO: 40)-[K(PYA)] (herein referred to as BCY17224);
A-(SEQ ID NO: 41)-A (herein referred to as BCY16135);
A-(SEQ ID NO: 42)-A (herein referred to as BCY16136);
A-(SEQ ID NO: 43)-A (herein referred to as BCY16137);
A-(SEQ ID NO: 44)-A (herein referred to as BCY16138);
A-(SEQ ID NO: 45)-A (herein referred to as BCY16139);
A-(SEQ ID NO: 46)-A (herein referred to as BCY16140);
A-(SEQ ID NO: 47)-A (herein referred to as BCY16141);
A-(SEQ ID NO: 48)-A (herein referred to as BCY16142);
A-(SEQ ID NO: 49)-A (herein referred to as BCY16143);
A-(SEQ ID NO: 50)-A (herein referred to as BCY16144);
A-(SEQ ID NO: 51)-A (herein referred to as BCY16145);
A-(SEQ ID NO: 52)-A-Sar₆-KFI (herein referred to as BCY15025);
A-(SEQ ID NO: 53)-A-Sar₆-KFI (herein referred to as BCY15028);
A-(SEQ ID NO: 54)-A-Sar₆-KFI (herein referred to as BCY15030);
A-(SEQ ID NO: 55)-A-Sar₆-KFI (herein referred to as BCY15035);
A-(SEQ ID NO: 56)-A-Sar₆-KFI (herein referred to as BCY15117);
A-(SEQ ID NO: 57)-A-Sar₆-KFI (herein referred to as BCY15122);
A-(SEQ ID NO: 58)-A-Sar₆-KFI (herein referred to as BCY15123); and
A-(SEQ ID NO: 59)-A-Sar₆-KFI (herein referred to as BCY15124),

wherein PYA represents pentynoic acid, Sar represents sarcosine and FI represents fluorescein.

CD16a Binding Bicyclic Peptides

In one embodiment, the bicyclic peptide is specific for (i.e. binds to) an Fc receptor present on the NK cell surface. In a further embodiment, the bicyclic peptide is specific for (i.e. binds to) a low-affinity Fc gamma receptor (FcγR) selected from FcγRIIA, FcγRIIB, FcγRIIC, FcγRIIIA, and FcγRIIIB. In a yet further embodiment, the bicyclic peptide is specific for (i.e. binds to) FcγRIIIA (also known as CD16a).

Fc receptors are expressed on the surface of many leukocytes. In humans, five classic low-affinity Fc gamma receptors (FcγRs) (FcγRIIA, FcγRIIB, FcγRIIC, FcγRIIIA, and FcγRIIIB) bind to the Fc portion of immunoglobulin G (IgG) and are mediators of inflammation via immune cell activation as well as inhibition (Muta et al. 1994, Ravetch et al. 2001). The FcγRIIIA (CD16a) is activated by engagement with the Fc portion of the IgG molecule and is critical for the antibody-dependent cell cytotoxicity (ADCC) process. ADCC is a mechanism in which antigen-specific antibodies direct NK cells to kill the antigen-expressing cancer cells (Arnould et al. 2006). Playing a vital role in the anti-tumour effects of IgG1 mAbs, several studies have shown that part of the anti-tumor effect of trastuzumab, a human IgG1 anti-human epidermal growth factor receptor 2 (HER-2) antibody, as well as the EGFR-antibody cetuximab in metastatic colorectal patients, is through ADCC (Zhang et al. 2007, 2020, Wu et al. 2003). Similar results can be described for rituximab, a chimeric IgG1 mAb for B-cell differentiation antigen CD20 (Manches et al. 2003, Clynes et al. 2000). The usefulness of CD16 expression in directing immune cells to promote tumour cell killing has been illustrated in overexpression studies. Through retroviral transduction of Ig-Fc, the expression of IgFc on the surface of B16 melanoma cells lead to tumor killing in vivo (Riddle et al. 2005). The role of FcγR engagement for directing NK cell to tumors has also been illustrated in studies whereby chimeric antigen receptor T cells express CD16 scFv and are directed to antibody coated tumor cells. The introduction of CD16-CarT in in vivo models to EGFR or CD20 tumor-bearing mice treated with cetuximab or rituximab enhanced immune cell targeting and ADCC killing thereby eradicating evasive tumors (Rataj et al. 2019, Caratelli et al. 2017).

The contributions of FcγR genes to autoimmune diseases have attracted substantial attention, and functional FcγR polymorphisms have been reported to play important roles in the pathogenesis of autoimmune diseases (Salmon et al. 2001, Morgan et al. 2003, Wu et al. 1997). FcγR-knockout mouse models indicate that both activating and inhibitory FcγRs influence the development of autoimmune diseases (Nabbe et al. 2003, Kleinau et al 2000, Bolland et al. 2000). The variations in FcγR expression significantly affect IgG immune complex-mediated signal thresholds. Notably, proinflammatory and anti-inflammatory cytokines could modulate the expression levels of activating and inhibitory FcγRs that affect the threshold immune cell response to IgG immune complexes (Pricop et al. 2001, Boruchov et al. 2005). The FcγRIIIA and FcγRIIIB copy number variations have been associated with systemic lupus erythematosus (SLE) and rheumatoid arthritis (RA) in Taiwanese patients (Chen et al. 2014). A high FcγRIIIA copy number was demonstrated to be a risk factor for SLE. Higher frequencies of cytokine-producing FcγRIIIA-positive dendritic cells (DCs) were observed in SLE patients, particularly in those with active disease, suggesting that FcγRIIIA-mediated inflammatory cytokine production in DCs might contribute to disease pathogenesis (Henriques et al. 2012). It is thought that the higher density of activating FcγRIIIA on the surface of immune cells (NK cells, monocytes, DCs, macrophages, and subsets of T cells) could tip the delicate balance of immune responses toward intense inflammation, which may result in the development of SLE (Chen et al. 2014). A correlation has been demonstrated between a low FcγRIIIA copy number and low CD16A expression on NK cells, suggesting that FcγRIIIA copy number has physiologic implications in NK cell functions. Most importantly, FcγRIIIA deficiency is associated with 2 distinct autoimmune diseases (SLE and RA), suggesting that defective FcγRIIIA functions may represent a common risk factor for various autoimmune diseases. The disease associations suggest that modulation of FcγRIIIA function may be an important therapeutic target for lupus nephritis (Chen et al. 2014).

Therapeutic innovations have provided some hope as to a means to block inflammation in autoimmune disease through FcγR interactions. Previous studies support the potential of FcγR inhibition as a therapeutic strategy to inhibit immune complex-mediated events in autoimmune diseases (Clarkson et al. 1986, Flaherty et al. 2012). The IgG Fc fragment by itself has demonstrated good efficacy in animal models of RA and in humans with thrombocytopenia (ITP) or Kawasaki disease (Anthony et al. 2008, Debré M et al. 1993, Hsu et al. 1993). Multivalent Fc constructs have been shown to inhibit immune complex processes in murine models of thrombocytopenia and arthritis (Ortiz et al. 2016). Furthermore, the infusion of soluble FcγR3a and 2a can inhibit immune complex triggered inflammation in the murine lupus model (Li et al. 2014). The blockade of immune complex formation on NK cells is an avenue to explore for the decreased activation of inflammation and thus autoimmune diseases.

In a further embodiment, said loop sequences comprise 3, 4, 5, 6, 7 or 8 amino acids. In a yet further embodiment, said loop sequences comprise 3, 4, 5, 7 or 8 amino acids.

In one embodiment, said loop sequences comprise three cysteine residues separated by two loop sequences the first of which consists of 4 amino acids and the second of which consists of 8 amino acids.

In a further embodiment, said loop sequences comprise three cysteine residues separated by two loop sequences the first of which consists of 4 amino acids and the second of which consists of 8 amino acids and the bicyclic peptide ligand comprises an amino acid sequence which is:

| (SEQ ID NO: 265) | |
|---|---|
| | CᵢTWPYCᵢᵢKSWGDVQDCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 4 amino acids and the second of which consists of 8 amino acids, the molecular scaffold is TATB and the bicyclic peptide ligand additionally comprises N- and/or C-terminal additions and comprises an amino acid sequence which is selected from:
A-(SEQ ID NO: 265)-A (herein referred to as BCY15044); and
A-(SEQ ID NO: 265)-AGAAAE (herein referred to as BCY19416).

In an alternative embodiment, said loop sequences comprise three cysteine residues separated by two loop sequences the first of which consists of 5 amino acids and the second of which consists of 4 amino acids.

In a further embodiment, said loop sequences comprise three cysteine residues separated by two loop sequences the first of which consists of 5 amino acids and the second of which consists of 4 amino acids and the bicyclic peptide ligand comprises an amino acid sequence which is selected from:

| (SEQ ID NO: 60) | |
|---|---|
| | CᵢPPTVYCᵢᵢHPLDCᵢᵢᵢ; |
| (SEQ ID NO: 169) | |
| | CᵢPPAVYCᵢᵢHPLDCᵢᵢᵢ; |
| (SEQ ID NO: 170) | |
| | CᵢPPEVFCᵢᵢHPLDCᵢᵢᵢ; |
| (SEQ ID NO: 171) | |
| | CᵢPPEVYCᵢᵢHPLDCᵢᵢᵢ; |
| (SEQ ID NO: 172) | |
| | CᵢPPEVWCᵢᵢHPLDCᵢᵢᵢ; |
| (SEQ ID NO: 173) | |
| | CᵢPPQVYCᵢᵢHPLDCᵢᵢᵢ; |
| | and |
| (SEQ ID NO: 174) | |
| | CᵢPPEVWCᵢᵢHPLDCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three cysteine residues separated by two loop sequences the first of which consists of 5 amino acids and the second of which consists of 4 amino acids and the bicyclic peptide ligand comprises an amino acid sequence which is:

| (SEQ ID NO: 60) | |
|---|---|
| | CᵢPPTVYCᵢᵢHPLDCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 5 amino acids and the second of which consists of 4 amino acids, the molecular scaffold is TATA and the bicyclic peptide ligand additionally comprises N- and/or C-terminal additions and comprises an amino acid sequence which is selected from:
A-(SEQ ID NO: 60)-A (herein referred to as BCY11808);
A-(SEQ ID NO: 60)-A-[K(PYA)] (herein referred to as BCY15685);
A-(SEQ ID NO: 60)-A-[Sar6]-[KBiot] (herein referred to as BCY12621);
A-(SEQ ID NO: 169)-A (herein referred to as BCY11673);
A-(SEQ ID NO: 170)-A (herein referred to as BCY11809);
A-(SEQ ID NO: 171)-A (herein referred to as BCY11810);
A-(SEQ ID NO: 172)-A (herein referred to as BCY11811)
A-(SEQ ID NO: 173)-A (herein referred to as BCY11812); and
A-(SEQ ID NO: 174)-A-[Sar6]-[KBiot] (herein referred to as BCY12622);

wherein Sar represents sarcosine and Biot represents biotin.

In a still yet further embodiment, said loop sequences comprise three reactive groups separated by two loop sequences the first of which consists of 5 amino acids and the second of which consists of 4 amino acids, the molecular scaffold is TATA and the bicyclic peptide ligand additionally comprises N- and/or C-terminal additions and comprises an amino acid sequence which is:
A-(SEQ ID NO: 60)-A-[K(PYA)] (herein referred to as BCY15685).

In an alternative embodiment, said loop sequences comprise three cysteine residues separated by two loop sequences the first of which consists of 6 amino acids and the second of which consists of 3 amino acids.

In a further embodiment, said loop sequences comprise three cysteine residues separated by two loop sequences the first of which consists of 6 amino acids and the second of which consists of 3 amino acids and the bicyclic peptide ligand comprises an amino acid sequence which is:

| (SEQ ID NO: 266) | |
|---|---|
| | CᵢDWWDPGCᵢᵢTYFCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 6 amino acids and the second of which consists of 3 amino acids, the molecular scaffold is TATB and the bicyclic peptide ligand additionally comprises N- and/or C-terminal additions and comprises an amino acid sequence which is selected from:
A-(SEQ ID NO: 266)-A (herein referred to as BCY15045); and
A-(SEQ ID NO: 266)-AGAAAE (herein referred to as BCY19417).

In an alternative embodiment, said loop sequences comprise three cysteine residues separated by two loop sequences the first of which consists of 7 amino acids and the second of which consists of 3 amino acids.

In a further embodiment, said loop sequences comprise three cysteine residues separated by two loop sequences the first of which consists of 7 amino acids and the second of which consists of 3 amino acids and the bicyclic peptide ligand comprises an amino acid sequence which is selected from:

| (SEQ ID NO: 61) | |
|---|---|
| | CᵢRWSVEDPCᵢᵢGAWCᵢᵢᵢ; |
| (SEQ ID NO: 175) | |
| | CᵢRWS[tBuGly]EDPCᵢᵢGAWCᵢᵢᵢ; |
| (SEQ ID NO: 176) | |
| | CᵢRWSVED[HyP]CᵢᵢGAWCᵢᵢᵢ; |
| (SEQ ID NO: 177) | |
| | CᵢRWSVED[Aze]CᵢᵢGAWCᵢᵢᵢ; |
| (SEQ ID NO: 178) | |
| | CᵢRWSVEDPCᵢᵢ[dA]AWCᵢᵢᵢ; |
| (SEQ ID NO: 179) | |
| | CᵢRWSVE[Gla]PCᵢᵢGAWCᵢᵢᵢ; |
| (SEQ ID NO: 180) | |
| | CᵢRWSVED[Aze]CᵢᵢGAWCᵢᵢᵢ; |
| (SEQ ID NO: 181) | |
| | CᵢMWIRSDPCᵢᵢGAWCᵢᵢᵢ; |
| (SEQ ID NO: 182) | |
| | CᵢRWYPDDPCᵢᵢGAWCᵢᵢᵢ; |
| (SEQ ID NO: 183) | |
| | CᵢEWHISEPCᵢᵢGAWCᵢᵢᵢ; |
| (SEQ ID NO: 184) | |
| | CᵢNWHVYEPCᵢᵢGAWCᵢᵢᵢ; |
| (SEQ ID NO: 185) | |
| | CᵢRWHFSEPCᵢᵢGAWCᵢᵢᵢ; |
| (SEQ ID NO: 186) | |
| | CᵢRWNIDDPCᵢᵢGAWCᵢᵢᵢ; |
| (SEQ ID NO: 187) | |
| | CᵢMWIRSDPCᵢᵢGAWCᵢᵢᵢ; |
| (SEQ ID NO: 188) | |
| | CᵢRWYPDDPCᵢᵢGAWCᵢᵢᵢ; |
| (SEQ ID NO: 189) | |
| | CᵢEWHISEPCᵢᵢGAWCᵢᵢᵢ; |
| (SEQ ID NO: 190) | |
| | CᵢNWHVYEPCᵢᵢGAWCᵢᵢᵢ; |
| (SEQ ID NO: 191) | |
| | CᵢRWHFSEPCᵢᵢGAWCᵢᵢᵢ; |
| (SEQ ID NO: 192) | |
| | CᵢRWNIDDPCᵢᵢGAWCᵢᵢᵢ; |
| (SEQ ID NO: 193) | |
| | CᵢR[2MeTrp]SVEDPCᵢᵢGAWCᵢᵢᵢ; |
| (SEQ ID NO: 194) | |
| | CᵢR[6MeTrp]SVEDPCᵢᵢGAWCᵢᵢᵢ; |
| (SEQ ID NO: 195) | |
| | CᵢR[6FTrp]SVEDPCᵢᵢGAWCᵢᵢᵢ; |
| (SEQ ID NO: 196) | |
| | CᵢR[5FTrp]SVEDPCᵢᵢGAWCᵢᵢᵢ; |
| (SEQ ID NO: 197) | |
| | CᵢR[6CITrp]SVEDPCᵢᵢGAWCᵢᵢᵢ; |
| (SEQ ID NO: 198) | |
| | CᵢR[Trp(S)]SVEDPCᵢᵢGAWCᵢᵢᵢ; |
| (SEQ ID NO: 199) | |
| | CᵢR[AzaTrp]SVEDPCᵢᵢGAWCᵢᵢᵢ; |
| (SEQ ID NO: 200) | |
| | CᵢRWSVEDPCᵢᵢGA[2MeTrp]Cᵢᵢᵢ; |
| (SEQ ID NO: 201) | |
| | CᵢRWSVEDPCᵢᵢGA[6FTrp]Cᵢᵢᵢ; |
| (SEQ ID NO: 202) | |
| | CᵢRWSVEDPCᵢᵢGA[5FTrp]Cᵢᵢᵢ; |
| (SEQ ID NO: 203) | |
| | CᵢRWSVEDPCᵢᵢGA[4MeoTrp]Cᵢᵢᵢ; |
| (SEQ ID NO: 204) | |
| | CᵢRWSVEDPCᵢᵢGA[5MeoTrp]Cᵢᵢᵢ; |
| (SEQ ID NO: 205) | |
| | CᵢRWSVEDPCᵢᵢGA[Trp(S)]Cᵢᵢᵢ; |
| (SEQ ID NO: 206) | |
| | CᵢRWSVEDPCᵢᵢGA[DOPA]Cᵢᵢᵢ; |
| (SEQ ID NO: 207) | |
| | CᵢRWYPDDPCᵢᵢGAWCᵢᵢᵢ; |
| (SEQ ID NO: 208) | |
| | CᵢRWHFSEPCᵢᵢGAWCᵢᵢᵢ; |
| (SEQ ID NO: 209) | |
| | CᵢR[2MeTrp]HFSEPCᵢᵢGAWCᵢᵢᵢ; |
| (SEQ ID NO: 210) | |
| | CᵢRWYFSEPCᵢᵢGAWCᵢᵢᵢ; |
| (SEQ ID NO: 211) | |
| | CᵢRWSFSEPCᵢᵢGAWCᵢᵢᵢ; |
| (SEQ ID NO: 212) | |
| | CᵢRWHPSEPCᵢᵢGAWCᵢᵢᵢ; |
| (SEQ ID NO: 213) | |
| | CᵢRWYPSEPCᵢᵢGAWCᵢᵢᵢ; |
| (SEQ ID NO: 214) | |
| | CᵢRWHPDEPCᵢᵢGAWCᵢᵢᵢ; |
| (SEQ ID NO: 215) | |
| | CᵢRWHPEEPCᵢᵢGAWCᵢᵢᵢ; |
| (SEQ ID NO: 216) | |
| | CᵢRWHFDEPCᵢᵢGAWCᵢᵢᵢ; |
| (SEQ ID NO: 217) | |
| | CᵢRWHFDDPCᵢᵢGAWCᵢᵢᵢ; |
| (SEQ ID NO: 218) | |
| | CᵢRWYFDEPCᵢᵢGAWCᵢᵢᵢ; |
| (SEQ ID NO: 219) | |
| | CᵢRW[His1Me]FSEPCᵢᵢGAWCᵢᵢᵢ; |
| (SEQ ID NO: 220) | |
| | CᵢRW[His3Me]FSEPCᵢᵢGAWCᵢᵢᵢ; |
| (SEQ ID NO: 221) | |
| | CᵢRW[4MeoPhe]FSEPCᵢᵢGAWCᵢᵢᵢ; |
| (SEQ ID NO: 222) | |
| | CᵢRW[DOPA]FSEPCᵢᵢGAWCᵢᵢᵢ; |
| (SEQ ID NO: 223) | |
| | CᵢRW[26DiMeTyr]FSEPCᵢᵢGAWCᵢᵢᵢ; |
| (SEQ ID NO: 224) | |
| | CᵢRW[3tBuTyr]FSEPCᵢᵢGAWCᵢᵢᵢ; |
| (SEQ ID NO: 225) | |
| | CᵢRW[4FPhe]FSEPCᵢᵢGAWCᵢᵢᵢ; |
| (SEQ ID NO: 226) | |
| | CᵢRWH[4MePhe]SEPCᵢᵢGAWCᵢᵢᵢ; |
| (SEQ ID NO: 227) | |
| | CᵢRWH[Aze]SEPCᵢᵢGAWCᵢᵢᵢ; |
| (SEQ ID NO: 228) | |
| | CᵢRWH[HyP]SEPCᵢᵢGAWCᵢᵢᵢ; |
| (SEQ ID NO: 229) | |
| | CᵢRWH[4tBuPhe]SEPCᵢᵢGAWCᵢᵢᵢ; |
| | (SEQ ID NO: 230, herein referred |
| | to as BCY21693 when complexed |
| | with TBMT) |
| | CᵢRWH[HPhe]SEPCᵢGAWCᵢᵢᵢ; |
| (SEQ ID NO: 231) | |
| | CᵢRWH[4PhePro]SEPCᵢᵢGAWCᵢᵢᵢ; |
| (SEQ ID NO: 232) | |
| | CᵢRWHF[HSer]EPCᵢᵢGAWCᵢᵢᵢ; |
| (SEQ ID NO: 233) | |
| | CᵢRWHFS[Gla]PCᵢᵢGAWCᵢᵢᵢ; |
| (SEQ ID NO: 234) | |
| | CᵢRWHFS[HGlu]PCᵢᵢGAWCᵢᵢᵢ; |
| (SEQ ID NO: 235) | |
| | CᵢRWHFSEPCᵢᵢ[dA]AWCᵢᵢᵢ; |
| (SEQ ID NO: 236) | |
| | CᵢRWYFSE[Aze]Cᵢᵢ[dA]AWCᵢᵢᵢ; |
| (SEQ ID NO: 237) | |
| | CᵢRWHFDE[Aze]Cᵢᵢ[dA]AWCᵢᵢᵢ; |
| (SEQ ID NO: 238) | |
| | CᵢRWYPSE[Aze]Cᵢᵢ[dA]AWCᵢᵢᵢ; |
| (SEQ ID NO: 239) | |
| | Cᵢ[HArg]WHFSEPCᵢᵢGAWCᵢᵢᵢ; |
| (SEQ ID NO: 240) | |
| | CᵢRWH[Cis-HyP]SEPCᵢᵢGAWCᵢᵢᵢ; |
| (SEQ ID NO: 241) | |
| | CᵢRWHFSEPCᵢᵢGAWCᵢᵢᵢ; |
| (SEQ ID NO: 242) | |
| | CᵢRWHFSEPCᵢᵢGAWCᵢᵢᵢ; |
| (SEQ ID NO: 243) | |
| | CᵢRWHFSEPCᵢᵢGAWCᵢᵢᵢ; |
| (SEQ ID NO: 267) | |
| | CᵢRWH[4BnPro]SEPCᵢᵢGAWCᵢᵢᵢ; |
| | and |
| (SEQ ID NO: 268) | |
| | CᵢRWH[HPhe]SE[Aze]CᵢᵢGAWCᵢᵢᵢ; |

wherein Cᵢ, Cᵢᵢ and Cᵢᵢᵢ represent first, second and third cysteine residues, respectively, wherein Gla represents γ-carboxyglutamic acid, Aze represents azetidine-2-carboxylic acid, 2MeTrp 10 represents 2-methyltryptophan, 6MeTrp represents 6-methyltryptophan, 6FTrp represents 6-fluorotryptophan, 5FTrp represents 5-fluorotryptophan, 6CITrp represents 6-Chloro-L-tryptophan, 5MeoTrp represents 5-Methoxy-L-tryptophan, Trp(S) represents 3-(3-Benzothienyl)-L-alanine, AzaTrp represents azatryptophan, 4MeoTrp represents 4-Methoxy-L-tryptophan, DOPA represents 3,4-Dihydroxyphenylalanine, His1Me represents 1-methylhistidine, His3Me represents 3-methylhistidine, 4MeoPhe represents 4-Methoxyphenylalanine, 26DiMeTyr represents 2,6-Dimethyl-L-tyrosine, 3tBuTyr represents 3-tert-butyltyrosine, 4FPhe represents 4-fluoro-L-phenylalanine, 4MePhe represents 4-methyl-phenylalanine, HyP represents hydroxyproline, 4tBuPhe represents 4-t-butyl-phenylalanine, HPhe represents homophenylalanine, 4PhePro represents (2S,4S)-4-PhenylProline, HSer represents homoserine, HGlu represents L-2-aminoadipic acid, dA represents D-Alanine, HArg represents homoarginine, tBuGly represents tert-butylglycine, Cis-HyP represents 4-cis-hydroxyproline, and 4BnPro represents (2S,4R)-4-benzyl-pyrrolidine-2-carboxylic acid or a pharmaceutically acceptable salt thereof.

In a yet further embodiment, said loop sequences comprise three cysteine residues separated by two loop sequences the first of which consists of 7 amino acids and the second of which consists of 3 amino acids and the bicyclic peptide ligand comprises an amino acid sequence which is:

| (SEQ ID NO: 61) | |
|---|---|
| | CᵢRWSVEDPCᵢᵢGAWCᵢᵢᵢ; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 7 amino acids and the second of which consists of 3 amino acids, the molecular scaffold is TBMT and the bicyclic peptide ligand additionally comprises N- and/or C-terminal additions and comprises an amino acid sequence which is selected from:
A-(SEQ ID NO: 61)-A (herein referred to as BCY14950);
Ac-A-(SEQ ID NO: 61)-A (herein referred to as BCY16599);
A-(SEQ ID NO: 61)-AGAAAE (herein referred to as BCY19040);
A-(SEQ ID NO: 61)-A-[K(PYA)] (herein referred to as BCY13883);
Ac-A-(SEQ ID NO: 61)-A-[K(PYA)] (herein referred to as BCY19596);
A-(SEQ ID NO: 175)-A (herein referred to as BCY14953);
A-(SEQ ID NO: 176)-A (herein referred to as BCY14954);
[PYA]-A-(SEQ ID NO: 177) (herein referred to as BCY14955);
A-(SEQ ID NO: 178)-A (herein referred to as BCY14956);
A-(SEQ ID NO: 179)-A (herein referred to as BCY16608);
A-(SEQ ID NO: 180)-A (herein referred to as BCY16610);
A-(SEQ ID NO: 181)-A (herein referred to as BCY19032);
A-(SEQ ID NO: 182)-A (herein referred to as BCY19033);
A-(SEQ ID NO: 183)-A (herein referred to as BCY19034);
A-(SEQ ID NO: 184)-A (herein referred to as BCY19035);
A-(SEQ ID NO: 185)-A (herein referred to as BCY19036);
A-(SEQ ID NO: 186)-A (herein referred to as BCY19037);
A-(SEQ ID NO: 187)-AGAAAE (herein referred to as BCY19038);
A-(SEQ ID NO: 188)-AGAAAE (herein referred to as BCY19039);
A-(SEQ ID NO: 189)-AGAAAE (herein referred to as BCY19041);
A-(SEQ ID NO: 190)-AGAAAE (herein referred to as BCY19042);
A-(SEQ ID NO: 191)-AGAAAE (herein referred to as BCY19043);
A-(SEQ ID NO: 192)-AGAAAE (herein referred to as BCY19044);
A-(SEQ ID NO: 193)-A (herein referred to as BCY19045);
A-(SEQ ID NO: 194)-A (herein referred to as BCY19046);
A-(SEQ ID NO: 195)-A (herein referred to as BCY19048);
A-(SEQ ID NO: 196)-A (herein referred to as BCY19049);
A-(SEQ ID NO: 197)-A (herein referred to as BCY19050);
A-(SEQ ID NO: 198)-A (herein referred to as BCY19053);
A-(SEQ ID NO: 199)-A (herein referred to as BCY19055);
A-(SEQ ID NO: 200)-A (herein referred to as BCY19057);
A-(SEQ ID NO: 201)-A (herein referred to as BCY19060);
A-(SEQ ID NO: 202)-A (herein referred to as BCY19061);
A-(SEQ ID NO: 203)-A (herein referred to as BCY19063);
A-(SEQ ID NO: 204)-A (herein referred to as BCY19064);
A-(SEQ ID NO: 205)-A (herein referred to as BCY19065);
A-(SEQ ID NO: 206)-A (herein referred to as BCY19068);
A-(SEQ ID NO: 207)-A-[K(PYA)] (herein referred to as BCY20360);
A-(SEQ ID NO: 208)-A-[K(PYA)] (herein referred to as BCY20361);
A-(SEQ ID NO: 209)-A (herein referred to as BCY20363);
A-(SEQ ID NO: 210)-A (herein referred to as BCY20364);
A-(SEQ ID NO: 211)-A (herein referred to as BCY20365);
A-(SEQ ID NO: 212)-A (herein referred to as BCY20366);
A-(SEQ ID NO: 213)-A (herein referred to as BCY20367);
A-(SEQ ID NO: 214)-A (herein referred to as BCY20368);
A-(SEQ ID NO: 215)-A (herein referred to as BCY20369);
A-(SEQ ID NO: 216)-A (herein referred to as BCY20370);
A-(SEQ ID NO: 217)-A (herein referred to as BCY20371);
A-(SEQ ID NO: 218)-A (herein referred to as BCY20372);
A-(SEQ ID NO: 219)-A (herein referred to as BCY20373);
A-(SEQ ID NO: 220)-A (herein referred to as BCY20374);
A-(SEQ ID NO: 221)-A (herein referred to as BCY20375);
A-(SEQ ID NO: 222)-A (herein referred to as BCY20376);
A-(SEQ ID NO: 223)-A (herein referred to as BCY20377);
A-(SEQ ID NO: 224)-A (herein referred to as BCY20378);
A-(SEQ ID NO: 225)-A (herein referred to as BCY20379);
A-(SEQ ID NO: 226)-A (herein referred to as BCY20380);
A-(SEQ ID NO: 227)-A (herein referred to as BCY20382);
A-(SEQ ID NO: 228)-A (herein referred to as BCY20383);
A-(SEQ ID NO: 229)-A (herein referred to as BCY20384);
A-(SEQ ID NO: 230)-A (herein referred to as BCY20385);
(SEQ ID NO: 230)-A (herein referred to as BCY21690);
Ac-(SEQ ID NO: 230)-A (herein referred to as BCY21691);
A-(SEQ ID NO: 230) (herein referred to as BCY21692);
Ac-(SEQ ID NO: 230) (herein referred to as BCY21694);
[Nle]-(SEQ ID NO: 230)-A (herein referred to as BCY21695);
Ac-[Nle]-(SEQ ID NO: 230)-A (herein referred to as BCY21696);
Ac-A-(SEQ ID NO: 230)-A (herein referred to as BCY21697);
A-(SEQ ID NO: 231)-A (herein referred to as BCY20386);
A-(SEQ ID NO: 232)-A (herein referred to as BCY20388);
A-(SEQ ID NO: 233)-A (herein referred to as BCY20389);
A-(SEQ ID NO: 234)-A (herein referred to as BCY20390);
A-(SEQ ID NO: 235)-A (herein referred to as BCY20392);
A-(SEQ ID NO: 236)-A (herein referred to as BCY20394);
A-(SEQ ID NO: 237)-A (herein referred to as BCY20395);
A-(SEQ ID NO: 238)-A (herein referred to as BCY20396);
A-(SEQ ID NO: 239)-A (herein referred to as BCY20397);
A-(SEQ ID NO: 240)-A (herein referred to as BCY20526);
Ac-A-(SEQ ID NO: 241)-A (herein referred to as BCY20527);
[Aib]-(SEQ ID NO: 242)-A (herein referred to as BCY20528);
A-(SEQ ID NO: 243)-[Aib] (herein referred to as BCY20529),
A-(SEQ ID NO: 267)-A (herein referred to as BCY20387); and
A-(SEQ ID NO: 268)-A (herein referred to as BCY21689);

wherein PYA represents pentynoic acid and Aib represents aminoisobutryic acid.

In a still yet further embodiment, said loop sequences comprise three reactive groups separated by two loop sequences the first of which consists of 7 amino acids and the second of which consists of 3 amino acids, the molecular scaffold is TBMT and the bicyclic peptide ligand additionally comprises N- and/or C-terminal additions and comprises an amino acid sequence which is:
A-(SEQ ID NO: 61)-A-[K(PYA)] (herein referred to as BCY13883),

wherein PYA represents pentynoic acid.

In an alternative embodiment, said loop sequences comprise three cysteine residues separated by two loop sequences the first of which consists of 8 amino acids and the second of which consists of 3 amino acids.

In a further embodiment, said loop sequences comprise three cysteine residues separated by two loop sequences the first of which consists of 8 amino acids and the second of which consists of 3 amino acids and the bicyclic peptide ligand comprises an amino acid sequence which is selected from:

| | (SEQ ID NO: 62; hereᵢn |
|---|---|
| | referred to as BCY16494 |
| | when complexed wᵢth TBMT); |
| | CᵢGLEELGPCᵢᵢSDLCᵢᵢᵢ |
| (SEQ ID NO: 244) | |
| | CᵢVGLLELGPCᵢᵢSDLCᵢᵢᵢ; |
| (SEQ ID NO: 245) | |
| | CᵢPGLEELGPCᵢᵢSDLCᵢᵢᵢ; |
| (SEQ ID NO: 246) | |
| | CᵢV[dA]LEELGPCᵢᵢSDLCᵢᵢᵢ; |
| (SEQ ID NO: 247) | |
| | CᵢVG[tBuAla]EELGPCᵢᵢSDLCᵢᵢᵢ; |
| (SEQ ID NO: 248) | |
| | CᵢVG[Cba]EELGPCᵢᵢSDLCᵢᵢᵢ; |
| (SEQ ID NO: 249) | |
| | CᵢVGFEELGPCᵢᵢSDLCᵢᵢᵢ; |
| (SEQ ID NO: 250) | |
| | CᵢVG[1Nal]EELGPCᵢᵢSDLCᵢᵢᵢ; |
| (SEQ ID NO: 251) | |
| | CᵢVG[2Nal]EELGPCᵢᵢSDLCᵢᵢᵢ; |
| (SEQ ID NO: 252) | |
| | CᵢVGLPELGPCᵢᵢSDLCᵢᵢᵢ; |
| (SEQ ID NO: 253) | |
| | CᵢVGL[tBuAla]ELGPCᵢᵢSDLCᵢᵢᵢ; |
| (SEQ ID NO: 254) | |
| | CᵢVGL[Cba]ELGPCᵢᵢSDLCᵢᵢᵢ; |
| (SEQ ID NO: 255) | |
| | CᵢVGL[Cha]ELGPCᵢᵢSDLCᵢᵢᵢ; |
| (SEQ ID NO: 256) | |
| | CᵢVGL[Nle]ELGPCᵢᵢSDLCᵢᵢᵢ; |
| (SEQ ID NO: 257) | |
| | CᵢVGLE[Gla]LGPCᵢᵢSDLCᵢᵢᵢ; |
| (SEQ ID NO: 258) | |
| | CᵢVGLEELG[Aze]CᵢᵢSDLCᵢᵢᵢ; |
| (SEQ ID NO: 259) | |
| | CᵢVGLEELGPCᵢᵢSD[tBuAla]Cᵢᵢᵢ; |
| (SEQ ID NO: 260) | |
| | CᵢVGLEELGPCᵢᵢSD[Cba]Cᵢᵢᵢ; |
| (SEQ ID NO: 261) | |
| | CᵢVGLEELGPCᵢᵢSD[Cha]Cᵢᵢᵢ; |
| (SEQ ID NO: 262) | |
| | CᵢVG[HLeu]EELGPCᵢᵢSDLCᵢᵢᵢ; |
| (SEQ ID NO: 263) | |
| | CᵢVGL[HLeu]ELGPCᵢᵢSDLCᵢᵢᵢ; |
| | and |
| (SEQ ID NO: 264) | |
| | CᵢVGLE[HGlu]LGPCᵢᵢSDLCᵢᵢᵢ; |

wherein Cᵢ, Cᵢᵢ and Cᵢᵢᵢ represent first, second and third cysteine residues, respectively, and wherein dA represents D-Alanine, tBuAla represents β-t-Butyl-L-alanine, Cba represents cyclobutylalanine, 1NaI represents 1-napthylalanine, 2NaI represents 2-naphthylalanine, Cha represents 3-cyclohexyl-D-alanine, Ne represents norleucine, Gla represents γ-carboxyglutamic acid, Aze represents azetidine-2-carboxylic acid, HLeu represents homoleucine, HGlu represents L-2-aminoadipic acid, or a pharmaceutically acceptable salt thereof.

In a yet further embodiment, said loop sequences comprise three cysteine residues separated by two loop sequences the first of which consists of 8 amino acids and the second of which consists of 3 amino acids and the bicyclic peptide ligand comprises an amino acid sequence which is:

| (SEQ ID NO: 62) | |
|---|---|
| | CiVGLEELGPCiSDLCiii; |

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences the first of which consists of 8 amino acids and the second of which consists of 3 amino acids, the molecular scaffold is TBMT and the bicyclic peptide ligand additionally comprises N- and/or C-terminal additions and comprises an amino acid sequence which is selected from:
A-(SEQ ID NO: 62)-A (herein referred to as BCY16493);
Ac-(SEQ ID NO: 62) (herein referred to as BCY16495);
A-(SEQ ID NO: 62)-A-[K(PYA)] (herein referred to as BCY13886);
A-(SEQ ID NO: 244)-A-[K(PYA)] (herein referred to as BCY16165);
A-(SEQ ID NO: 245)-A (herein referred to as BCY16498);
A-(SEQ ID NO: 246)-A (herein referred to as BCY16499);
A-(SEQ ID NO: 247)-A (herein referred to as BCY16501);
A-(SEQ ID NO: 248)-A (herein referred to as BCY16502);
A-(SEQ ID NO: 249)-A (herein referred to as BCY16504);
A-(SEQ ID NO: 250)-A (herein referred to as BCY16505);
A-(SEQ ID NO: 251)-A (herein referred to as BCY16506);
A-(SEQ ID NO: 252)-A (herein referred to as BCY16507);
A-(SEQ ID NO: 253)-A (herein referred to as BCY16508);
A-(SEQ ID NO: 254)-A (herein referred to as BCY16509);
A-(SEQ ID NO: 255)-A (herein referred to as BCY16510);
A-(SEQ ID NO: 256)-A (herein referred to as BCY16511);
A-(SEQ ID NO: 257)-A (herein referred to as BCY16512);
A-(SEQ ID NO: 258)-A (herein referred to as BCY16520);
A-(SEQ ID NO: 259)-A (herein referred to as BCY16522);
A-(SEQ ID NO: 260)-A (herein referred to as BCY16523);
A-(SEQ ID NO: 261)-A (herein referred to as BCY16524);
A-(SEQ ID NO: 262)-A (herein referred to as BCY16527);
A-(SEQ ID NO: 263)-A (herein referred to as BCY16528); and
A-(SEQ ID NO: 264)-A (herein referred to as BCY16529),

wherein PYA represents pentynoic acid.

In a still yet further embodiment, said loop sequences comprise three reactive groups separated by two loop sequences the first of which consists of 8 amino acids and the second of which consists of 3 amino acids, the molecular scaffold is TBMT and the bicyclic peptide ligand additionally comprises N- and/or C-terminal additions and comprises an amino acid sequence which is:
A-(SEQ ID NO: 62)-A-[K(PYA)] (herein referred to as BCY13886);

wherein PYA represents pentynoic acid.

In a further embodiment, the pharmaceutically acceptable salt is selected from the free acid or the sodium, potassium, calcium or ammonium salt.

Unless defined otherwise, all technical and scientific terms used herein have the same meaning as commonly understood by those of ordinary skill in the art, such as in the arts of peptide chemistry, cell culture and phage display, nucleic acid chemistry and biochemistry.

Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

Numbering

When referring to amino acid residue positions within the peptides of the invention, reactive groups, i.e. cysteine or penicillamine residues (Cᵢ, Penᵢ, Cᵢᵢ, Penᵢᵢ and Cᵢᵢᵢ and Penᵢᵢᵢ) are omitted from the numbering as they are invariant, therefore, the numbering of amino acid residues within the peptides of the invention is referred to as below:

| (SEQ ID NO: 1) | |
|---|---|
| | -Cᵢ-N₁-L₂-Q₃-A₄-P₅-Cᵢᵢ-M₆-Q₇-T₈-G₉-K₁₀-V₁₁-Cᵢᵢᵢ. |

For the purpose of this description, all bicyclic peptides are assumed to be cyclised with TATA, TATB or TBMT and yielding a tri-substituted structure. Cyclisation with TATA, TATB or TBMT occurs on the first, second and third reactive groups (i.e. Cᵢ, Cᵢᵢ, Cᵢᵢᵢ).

Molecular Format

N- or C-terminal extensions to the bicycle core sequence are added to the left or right side of the sequence, separated by a hyphen. For example, an N-terminal biotin-G-Sar₅ tail would be denoted as:

| (SEQ ID NO: X) | |
|---|---|
| | [Biot]-G-[Sar₅]-A-. |

Inversed Peptide Sequences

In light of the disclosure in Nair et al. (2003) J. Immunol. 170(3), 1362-1373, it is envisaged that the peptide sequences disclosed herein would also find utility in their retro-inverso form. For example, the sequence is reversed (i.e. N-terminus become C-terminus and vice versa) and their stereochemistry is likewise also reversed (i.e. D-amino acids become L-amino acids and vice versa).

Peptide Ligand Definition

A peptide ligand, as referred to herein, refers to a peptide, peptidic or peptidomimetic covalently bound to a molecular scaffold. Typically, such peptides, peptidics or peptidomimetics comprise a peptide having natural or non-natural amino acids, two or more reactive groups (i.e. cysteine or penicillamine residues) which are capable of forming covalent bonds to the scaffold, and a sequence subtended between said reactive groups which is referred to as the loop sequence, since it forms a loop when the peptide, peptidic or peptidomimetic is bound to the scaffold. In the present case, the peptides, peptidics or peptidomimetics comprise at least three cysteine or penicillamine residues (referred to herein as Cᵢ, Penᵢ, Cᵢᵢ, Penᵢᵢ and Cᵢᵢᵢ and Penᵢᵢᵢ), and form at least two loops on the scaffold.

Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should in most circumstances demonstrate stability to plasma proteases, epithelial (“membrane-anchored”) proteases, gastric and intestinal proteases, lung surface proteases, intracellular proteases and the like. Protease stability should be maintained between different species such that a bicyclic peptide lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes; and
An optimal plasma half-life in the circulation. Depending upon the clinical indication and treatment regimen, it may be required to develop a bicyclic peptide with short or prolonged in vivo exposure times for the management of either chronic or acute disease states. The optimal exposure time will be governed by the requirement for sustained exposure (for maximal therapeutic efficiency) versus the requirement for short exposure times to minimise toxicological effects arising from sustained exposure to the agent.

Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g. —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

Multimeric Binding Complexes

According to a further aspect of the invention, there is provided a multimeric binding complex which comprises at least two bicyclic peptide ligands as defined herein, wherein said peptide ligands may be the same or different.

In one embodiment, the multimeric binding complexes comprise more than one bicyclic peptide which are the same (i.e. homomultimers). In an alternative embodiment, the multimeric binding complexes comprise bicyclic peptides which are different (i.e. heteromultimers).

In one embodiment, the multimeric binding complex additionally comprises a fluorophore.

In one embodiment, the multimeric binding complex comprises two bicyclic peptides which are the same (i.e. homodimers). Examples of suitable dimeric NKp46 homodimers are shown in Table A below:

TABLE A
Dimeric NKp46 Multimeric Binding Complexes of the Invention

| Complex | NKp46 BCY | Attachment | | Fluoro- |
|---|---|---|---|---|
| No. | No. | Point | Linker | phore |
| BCY15663 | BCY15452 | C-term | N-(amine- | AF488 |
| | | D-Lys(PYA) | PEG₂)-N- | |
| | | | bis(PEG₃-azide) | |
| BCY15921 | BCY15686 | C-term | N-(amine- | AF488 |
| | | Lys(PYA) | PEG₂)-N- | |
| | | | bis(PEG₃-azide) | |
| BCY15922 | BCY15687 | C-term | N-(amine- | AF488 |
| | | Lys(PYA) | PEG₂)-N- | |
| | | | bis(PEG₃-azide) | |
| BCY15455 | BCY15013 | C-term | N-(amine- | AF488 |
| | | D-Lys(PYA) | PEG₂)-N- | |
| | | | bis(PEG₃-azide) | |
| BCY18043 | BCY18004 | C-term | N-(amine- | AF488 |
| | | Lys(PYA) | PEG₂)-N- | |
| | | | bis(PEG₃-azide) | |

Examples of suitable dimeric CD16a homodimers are shown in Table B below:

TABLE B
Dimeric CD16a Multimeric Binding Complexes of the Invention

| Complex | CD16a BCY | Attachment | | Fluoro- |
|---|---|---|---|---|
| No. | No. | Point | Linker | phore |
| BCY15914 | BCY13883 | C-term | N-(amine- | AF488 |
| | | Lys(PYA) | PEG₂)-N- | |
| | | | bis(PEG₃-azide) | |
| BCY15915 | BCY15685 | C-term | N-(amine- | AF488 |
| | | Lys(PYA) | PEG₂)-N- | |
| | | | bis(PEG₃-azide) | |
| BCY15916 | BCY13886 | C-term | N-(amine- | AF488 |
| | | Lys(PYA) | PEG₂)-N- | |
| | | | bis(PEG₃-azide) | |

Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with one or more replacement amino acids, such as an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group; modification of amino acids such as cysteine, lysine, glutamate/aspartate and tyrosine with suitable amine, thiol, carboxylic acid and phenol-reactive reagents so as to functionalise said amino acids; and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal residue is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal residue is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Ca-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine or penicillamine (Cᵢ or Penᵢ) and/or the C-terminal cysteine or penicillamine (Cᵢᵢᵢ or Penᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al. (2005) PNAS, 102(2), 413-418).

In one embodiment, the modified derivative comprises removal of any amino acid residues and substitution with alanines, such as D-alanines. This embodiment provides the advantage of identifying key binding residues and removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al., Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al., Curr. Pharmaceutical Design, (2010), 16, 3185-203, and Nestor et al., Curr. Medicinal Chem (2009), 16, 4399-418).

Isotopic Variations

The present invention includes all pharmaceutically acceptable (radio)isotope-labelled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulphur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labelled compounds of peptide ligands of the invention can generally be prepared by conventional techniques known to those skilled in the art or by processes analogous to those described in the accompanying Examples using an appropriate isotopically-labelled reagent in place of the non-labelled reagent previously employed.

Molecular Scaffold

In one embodiment, the molecular scaffold comprises a non-aromatic molecular scaffold. References herein to “non-aromatic molecular scaffold” refers to any molecular scaffold as defined herein which does not contain an aromatic (i.e. unsaturated) carbocyclic or heterocyclic ring system.

Suitable examples of non-aromatic molecular scaffolds are described in Heinis et al. (2014) Angewandte Chemie, International Edition 53(6) 1602-1606.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold is 1,1′,1″-(1,3,5-triazinane-1,3,5-triyl)triprop-2-en-1-one (also known as triacryloylhexahydro-s-triazine (TATA):

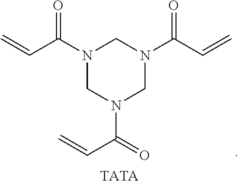

Thus, following cyclisation with the bicyclic peptides of the invention on the Cᵢ, Penᵢ, Cᵢᵢ, Penᵢᵢ, Cᵢᵢᵢ and Penᵢᵢᵢ cysteine or penicillamine residues, the molecular scaffold forms a tri-substituted 1,1′,1″-(1,3,5-triazinane-1,3,5-triyl)tripropan-1-one derivative of TATA having the following structure:

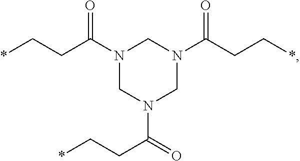

wherein * denotes the point of attachment of the three cysteine residues.

In an alternative embodiment, the molecular scaffold is 2,4,6-tris(bromomethyl)-s-triazine (TBMT):

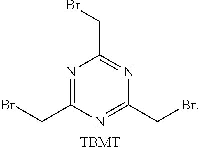

Thus, following cyclisation with the bicyclic peptides of the invention on the Cᵢ, Penᵢ, Cᵢᵢ, Penᵢᵢ, Cᵢᵢᵢ and Penᵢᵢᵢ cysteine or penicillamine residues, the molecular scaffold forms a tri-substituted derivative of TBMT having the following structure:

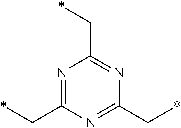 [SMILES: *Cc1nc(C*)nc(C*)n1]

wherein * denotes the point of attachment of the three cysteine residues.

In an alternative embodiment, the molecular scaffold is 1,3,5-tris(bromoacetyl) hexahydro-1, 3,5-triazine (TATB):

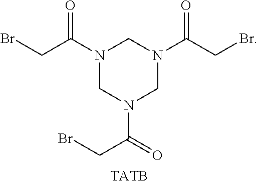

Thus, following cyclisation with the bicyclic peptides of the invention on the Cᵢ, Penᵢ, Cᵢᵢ, Penᵢᵢ, Cᵢᵢᵢ and Penᵢᵢᵢ cysteine or penicillamine residues, the molecular scaffold forms a tri-substituted 1,3,5-tris(bromoacetyl) hexahydro-1,3,5-triazine derivative of TATB having the following structure:

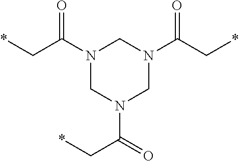 [SMILES: *CC(=O)N1CN(C(=O)C*)CN(C(=O)C*)C1]

wherein * denotes the point of attachment of the three cysteine residues.

Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al. (supra).

Thus, the invention also relates to the manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91(26):12544-8 or in Hikari et al. Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA, TATB or TBMT) could be added during the chemical synthesis of the first peptide so as to react with the three cysteine groups; a further cysteine or thiol could then be appended to the N- or C-terminus of the first peptide, so that this cysteine or thiol only reacted with a free cysteine or thiol of the second peptide, forming a disulphide-linked bicyclic peptide-peptide conjugate.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cyclosporine, methotrexate, adriamycin or cisplatinum and immunotoxins. Further examples of other agents which may be administered separately or in conjunction with the peptide ligands of the invention include cytokines, lymphokines, other hematopoietic factors, thrombolytic and anti-thrombotic factors. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered intravenously. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

Therapeutic Uses

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand or a drug conjugate as defined herein, for use in preventing, suppressing or treating a disease or disorder mediated by NK cells.

According to a further aspect of the invention, there is provided a method of preventing, suppressing or treating a disease or disorder mediated by NK cells, which comprises administering to a patient in need thereof the peptide ligand as defined herein.

In one embodiment, the disease or disorder mediated by NK cells is selected from inflammatory disorders, autoimmune disease and cancer. In a further embodiment, the disease or disorder mediated by NK cells is selected from: rheumatoid arthritis (RA), bone erosion, intraperitoneal abscesses, inflammatory bowel disease, allograft rejection, psoriasis, angiogenesis, atherosclerosis, asthma, multiple sclerosis, systemic lupus erythematosus (SLE), ocular surface disorders (such as dry eye), ankylosing spondylitis, psoriatic arthritis, cancer (such as multiple myeloma and breast cancer).

In a further embodiment, the disease or disorder mediated by NK cells is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### Examples

Materials and Methods

Preparation of Bicyclic Peptide Ligands (General Method)

Bicyclic peptides were synthesized on Rink amide resin using standard Fmoc (9-fluorenylmethyloxycarbonyl) solid-phase peptide synthesis, either by manual coupling (for large scale) or using a Biotage Syroll automated peptide synthesizer (for small scale). Following TFA-based cleavage from the resin, peptides were precipitated with diethyl ether and dissolved in 50:50 acetonitrile/water. The crude peptides (at ˜1 mM concentration) were then cyclized with 1.3 equiv. of the scaffold, using ammonium bicarbonate (100 mM) as a base. Completion of cyclization was determined by matrix-assisted laser desorption ionization time-of-flight (MALDI-TOF) or LC-MS. Once complete, the cyclization reaction was quenched using N-acetyl cysteine (10 equiv. with respect to the peptide), and the solutions were lyophilized. The residue was dissolved in an appropriate solvent and purified by RP-HPLC. Peptide fractions of sufficient purity and the correct molecular weight (verified by either MALDI-TOF and HPLC or LC-MS) were pooled and lyophilized. Concentrations were determined by UV absorption using the extinction coefficient at 280 nm, which was based on Trp/Tyr content.

All amino acids, unless noted otherwise, were used in the L-configurations.

### Examples

In general, some of the fluorescent dimeric bicyclic peptide complexes of the invention may be prepared in accordance with the following general method:

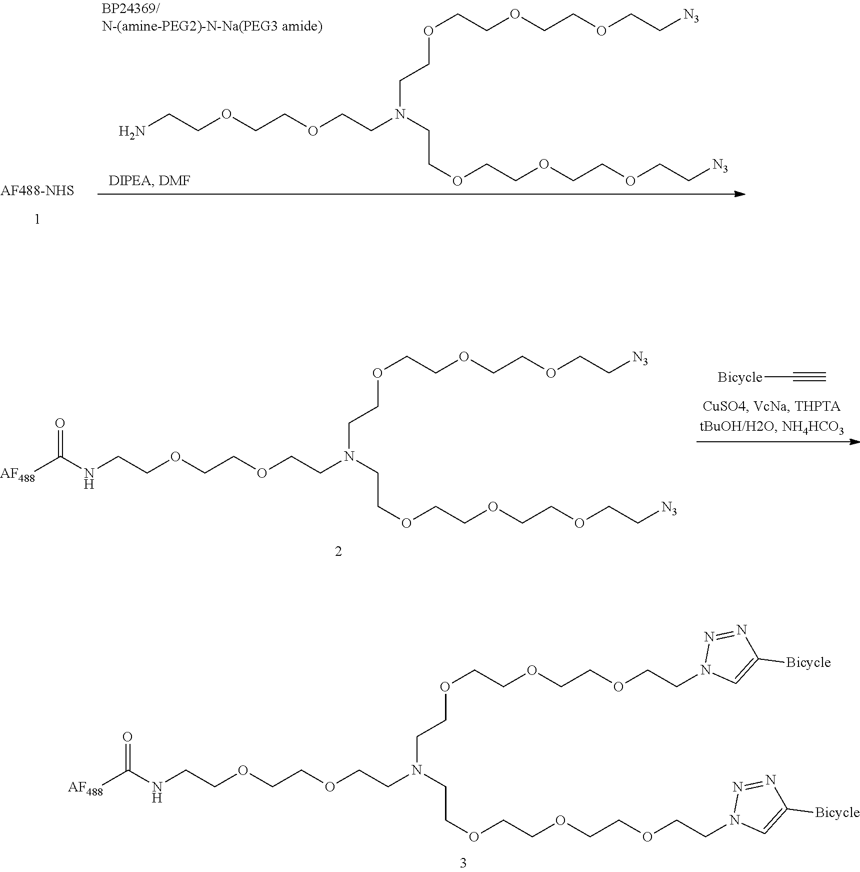

A mixture of BP24369 (1.0 eq) and AF488-NHS (1, 1.5 eq.) is dissolved in DMF. The pH of this solution is adjusted to 8 by dropwise addition of DIEA (3.0 eq). The reaction mixture is stirred at 25° C. for 1 hr, then a sample taken for analysis. If the reaction is incomplete, additional AF488-NHS is added and the mixture left to stir for a further 1 hr. Once complete, the reaction mixture is filtered to remove undissolved residue and the crude product purified by preparative HPLC to give intermediate 2.

A mixture of intermediate 2 (1.0 eq), Bicycle (2.1 eq) and THPTA (2.2 eq) are dissolved in degassed t-BuOH/H₂O (1:1), then aqueous CuSO₄ (0.4M, 2.2 eq) and VcNa (4.0 eq) is added. Finally, 0.2 M NH₄HCO₃ is added to adjust pH to 8. The reaction mixture is stirred for up to 16 hr under N₂ atmosphere. The reaction mixture is directly purified by prep-HPLC.

Fluorescently labelled dimeric bicyclic peptide complexes which were prepared using this method are listed below:

| NKp46 fluorescent dimers | CD16a fluorescent dimers |
|---|---|
| BCY15663 | BCY15914 |
| BCY15921 | BCY15915 |
| BCY15922 | BCY15916 |
| BCY15455 | |
| BCY18043 | |

More detailed experimental for selected bicyclic peptide multimeric complexes of the invention are provided herein below:

### Synthesis of Intermediate AF488-BP24369

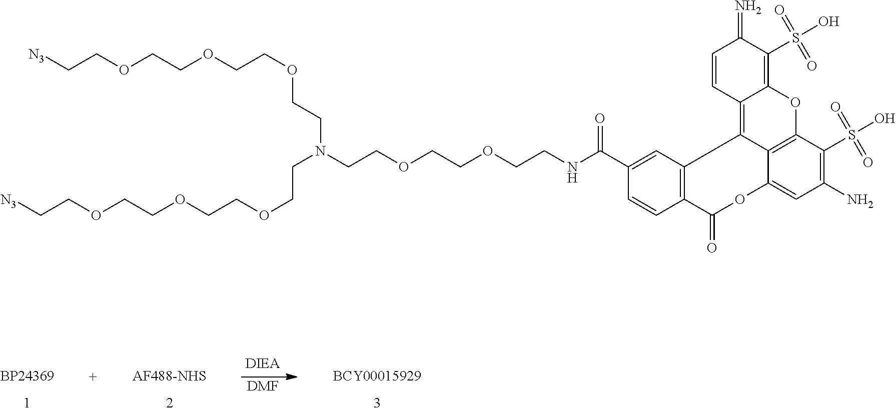

A mixture of compound 1 (10.0 mg, 18.2 μmol, 1.0 eq.), compound 2 (17.0 mg, 27.3 μmol, 1.5 eq.) was dissolved in DMF (0.4 mL). The pH of this solution was adjusted to 8 by dropwise addition of DIEA (15.8 μL, 181.60 μmol, 3.0 eq.). The reaction mixture was stirred at 25° C. for 1 hr. LC-MS showed BP24369 was not completely consumed, so additional compound 2 (17.0 mg, 27.24 μmol, 1.5 eq.) was added to the reaction mixture, which was stirred at 25° C. for 1 hr. LC-MS showed one main peak with desired m/z. The reaction mixture was filtered to remove undissolved residue. The crude product was purified by preparative HPLC, and AF488-BP24369 (10.0 mg, 8.90 μmol, 49.02% yield, 95.0% purity) was obtained as a red solid. Calculated MW: 1067.1, observed m/z: 1068.3 ([M+H]⁺), 534.7 ([M+2H]²⁺).

### Synthesis of BCY15663

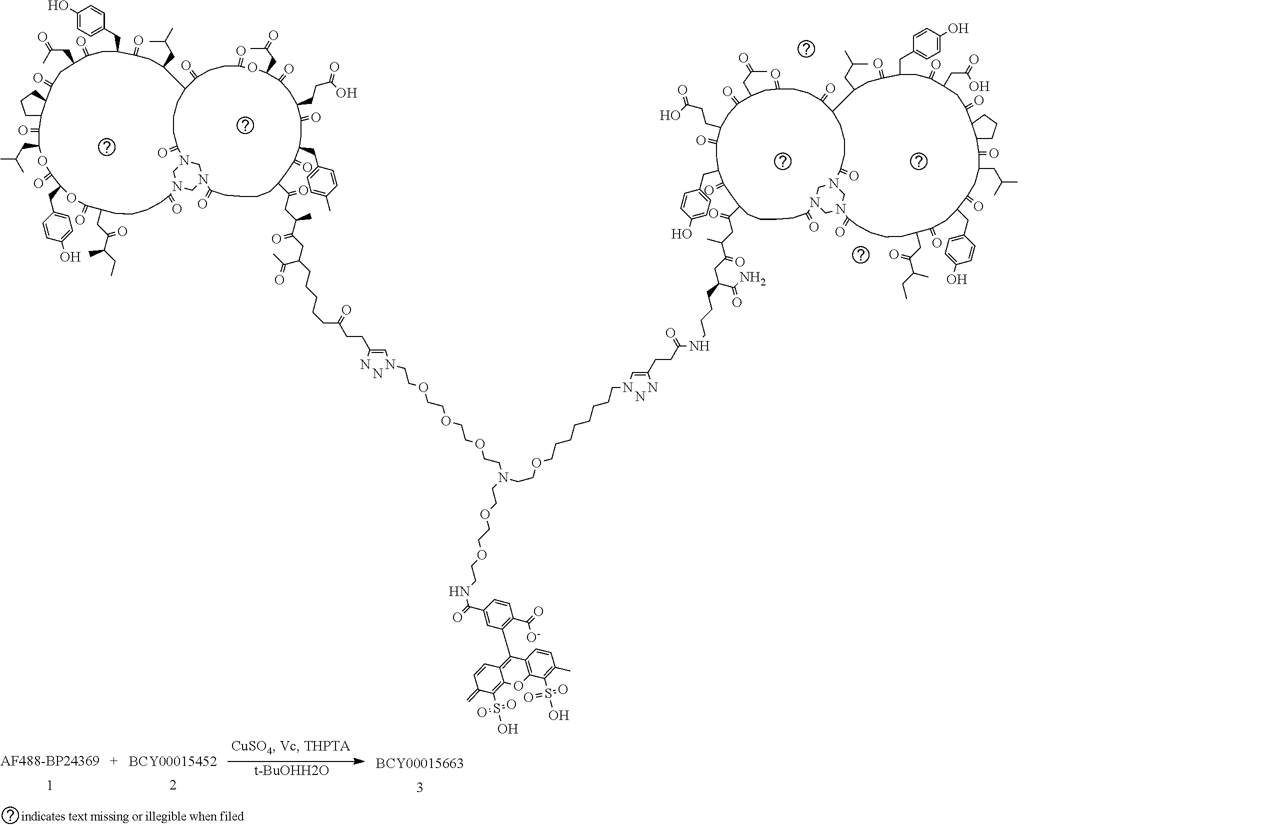

A mixture of compound 1 (3.0 mg, 2.81 μmol, 1.0 eq.), compound 2 (14.0 mg, 6.47 μmol, 2.3 eq.), and THPTA (1.2 mg, 2.81 μmol, 1.0 eq.) was dissolved in t-BuOH/H₂O (1:1, 0.5 mL), degassed and purged with N₂, and then an aqueous solution of CuSO₄ (0.4 M, 7.0 μL, 1.0 eq.) and Vc (2.0 mg, 11.25 μmol, 4.0 eq.) were added under N₂. The pH of this solution was adjusted to 8, and the solution turned light yellow. The reaction mixture was stirred at 25° C. for 1 hr under N₂ atmosphere. LC-MS showed AF488-BP24369 was consumed completely, some BCY15452 remained and one main peak with desired m/z was detected. The reaction mixture was filtered to remove undissolved residue. The crude product was purified by preparative HPLC, and BCY15663 (9.4 mg, 1.66 μmol, 59.06% yield, 96.4% purity) was obtained as a white solid. Calculated MW: 5377.9, observed m/z: 1345.5 ([M+4H]⁴⁺).

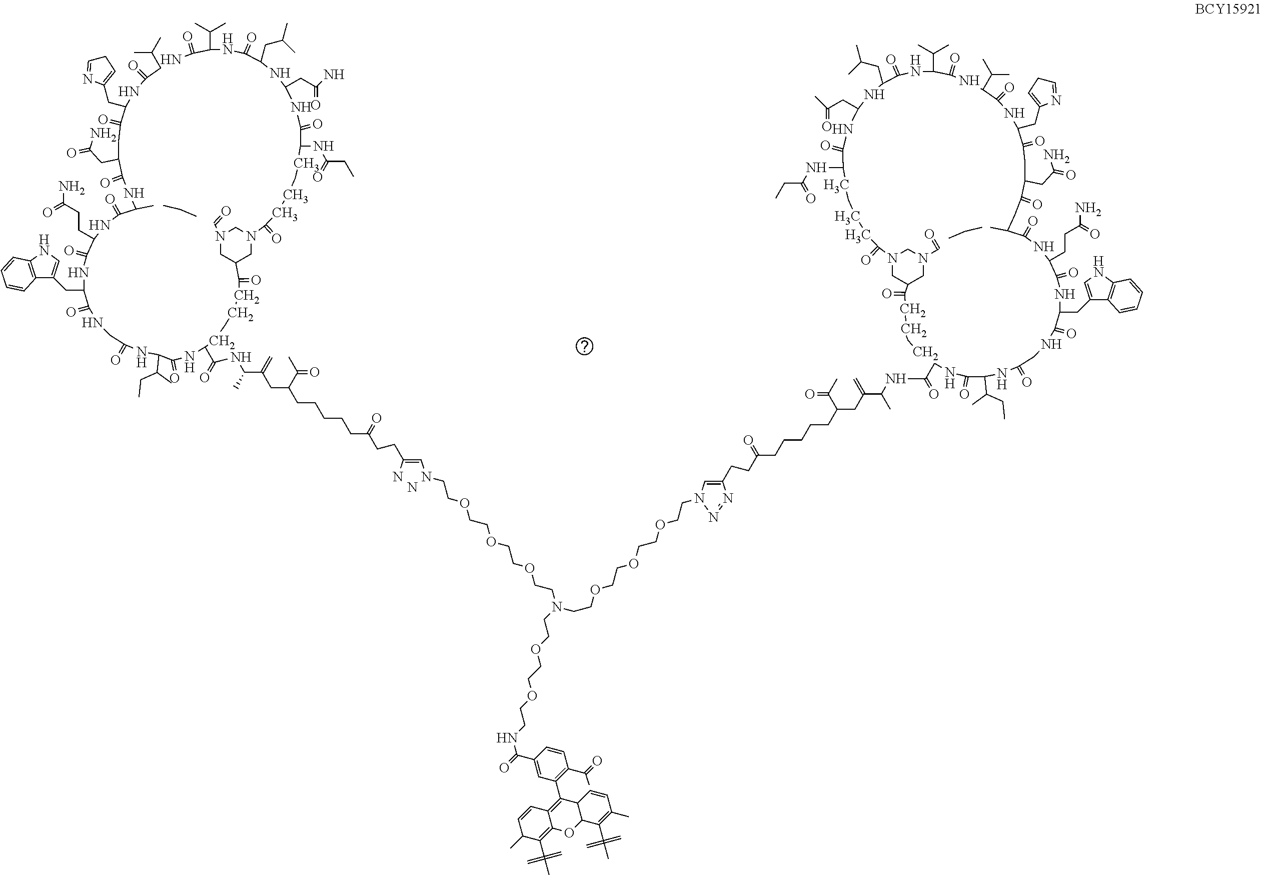

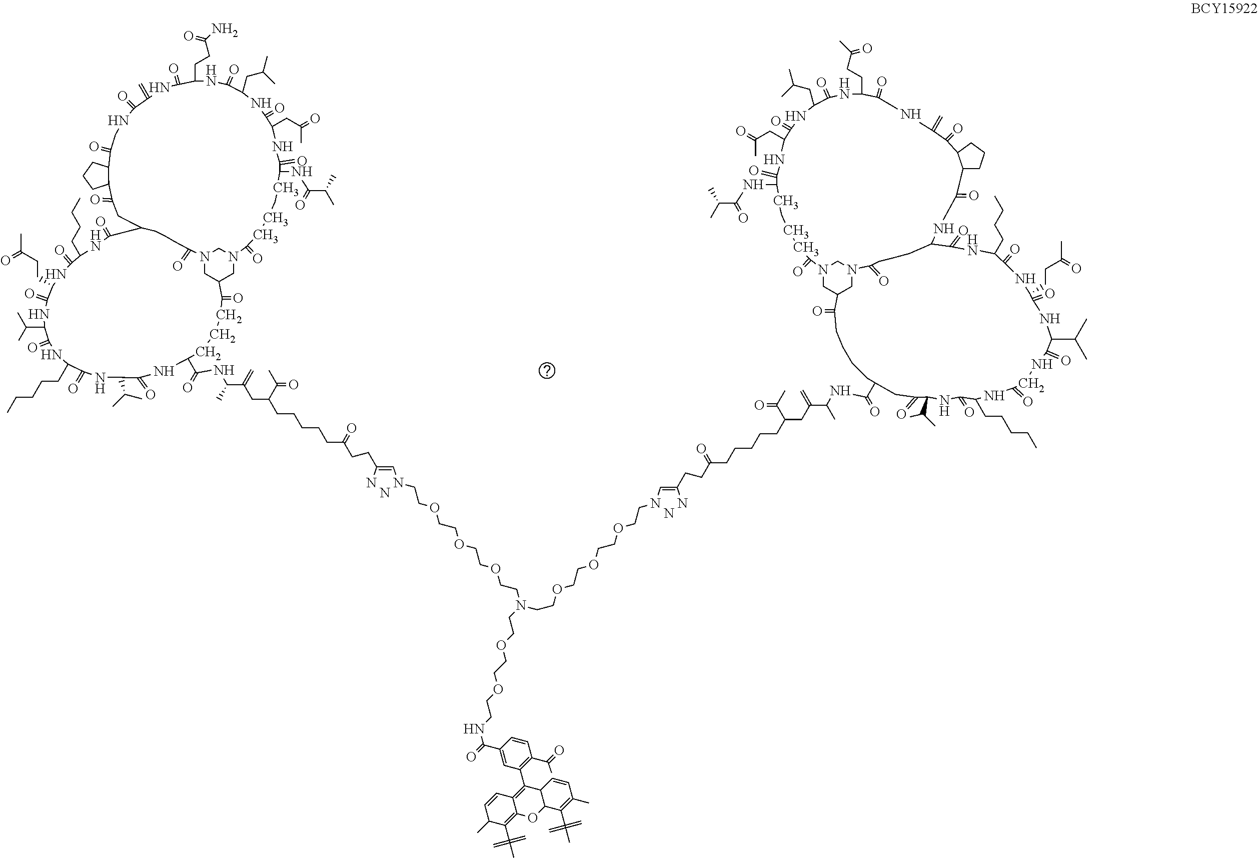

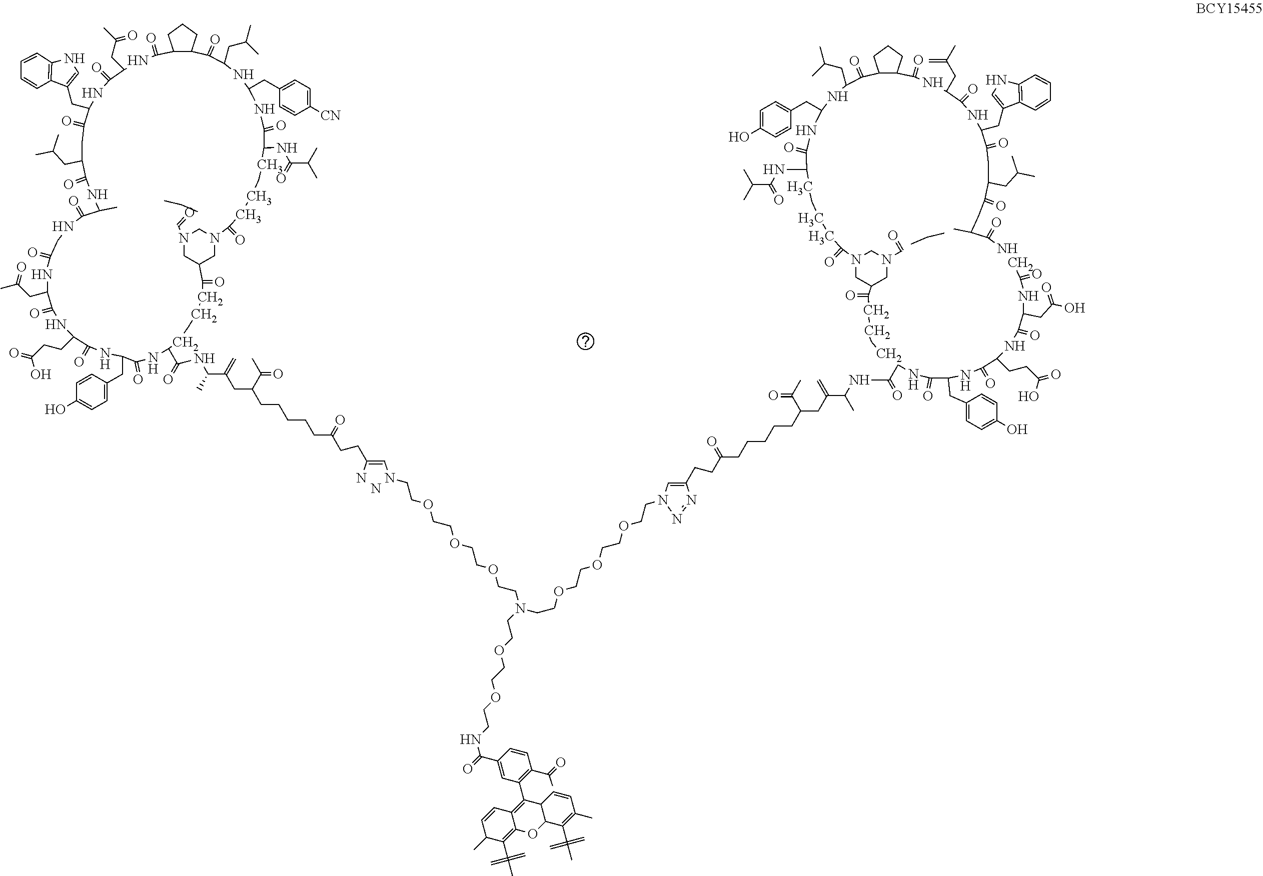

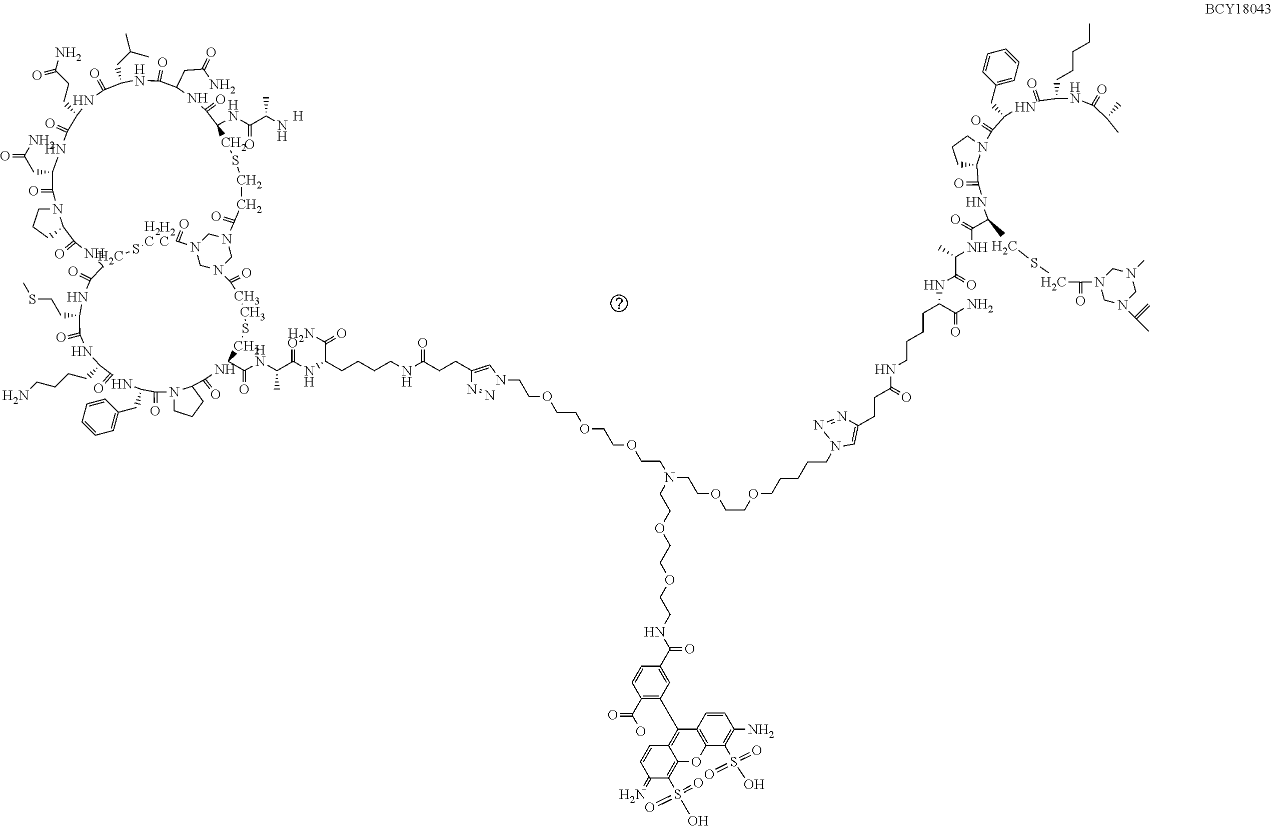

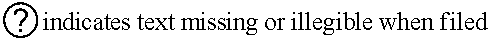

### Synthesis of BCY15916

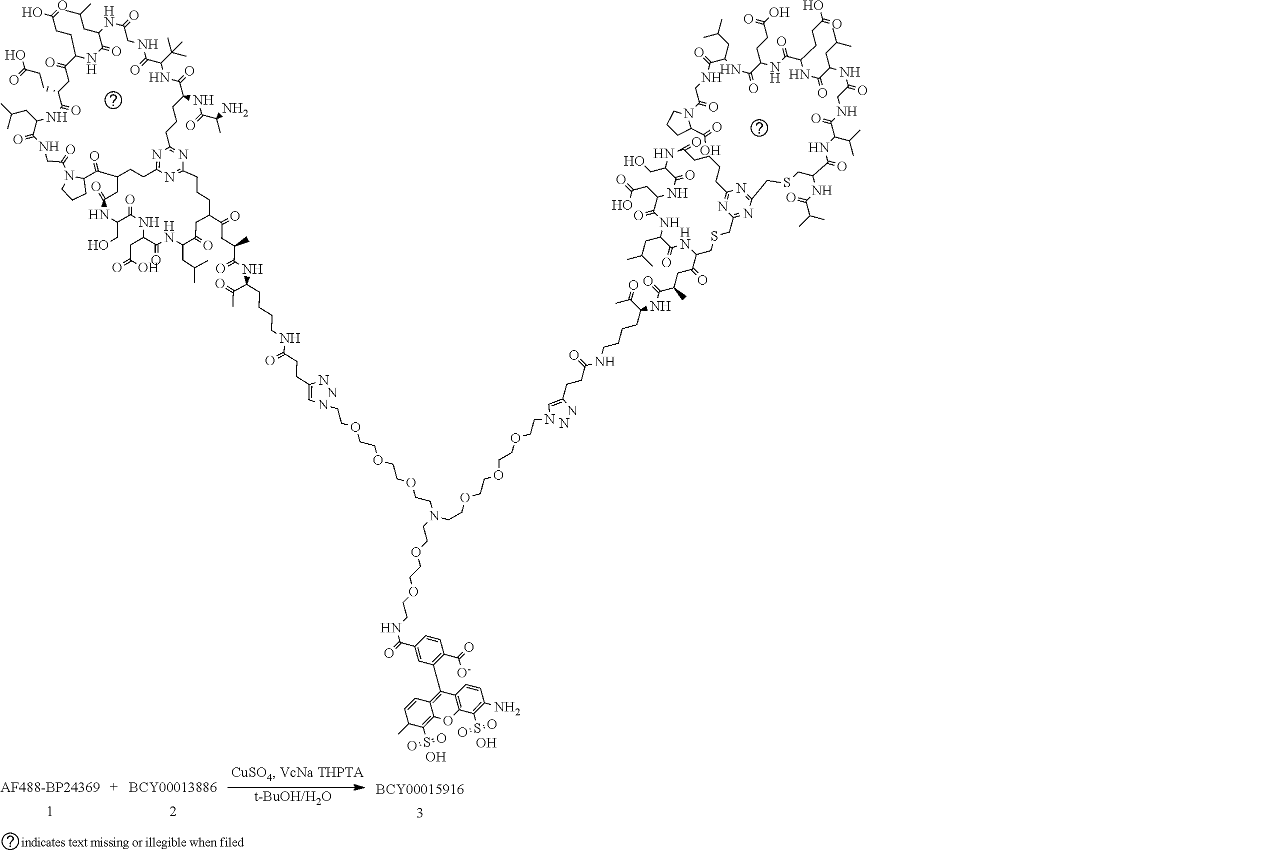

A mixture of compound 1 (2.0 mg, 1.87 μmol,

1.0 eq.), compound 2 (7.5 mg, 3.94 μmol, 2.1 eq.),

and THPTA (1.8 mg, 4.12 μmol, 2.2 eq.) was dissolved in t-BuOH/H₂O (1:1, 0.5 mL, degassed and purged with N₂), and then an aqueous solution of CuSO₄ (0.4 M, 10.3 μL, 2.2 eq.) and VcNa (1.5 mg, 7.5 μmol, 4.0 eq.) were added under N₂. The pH of this solution was adjusted to 8 by dropwise addition of 0.2 M NH₄HCO₃ (in 1:1 t-BuOH/H₂O), and the solution turned light yellow. The reaction mixture was stirred at 25-30° C. for 1 hr under an N₂ atmosphere. LC-MS showed compound 1 was consumed completely, and one main peak with desired m/z was detected. The reaction mixture was filtered to remove undissolved residue. The crude product was purified by preparative HPLC, and BCY15916 (1.6 mg, 0.30 μmol, 16.3% yield, 93.3% purity) was obtained as a white solid. Calculated MW: 4875.5, observed m/z: 1219.6 ([M+4H]⁴⁺).

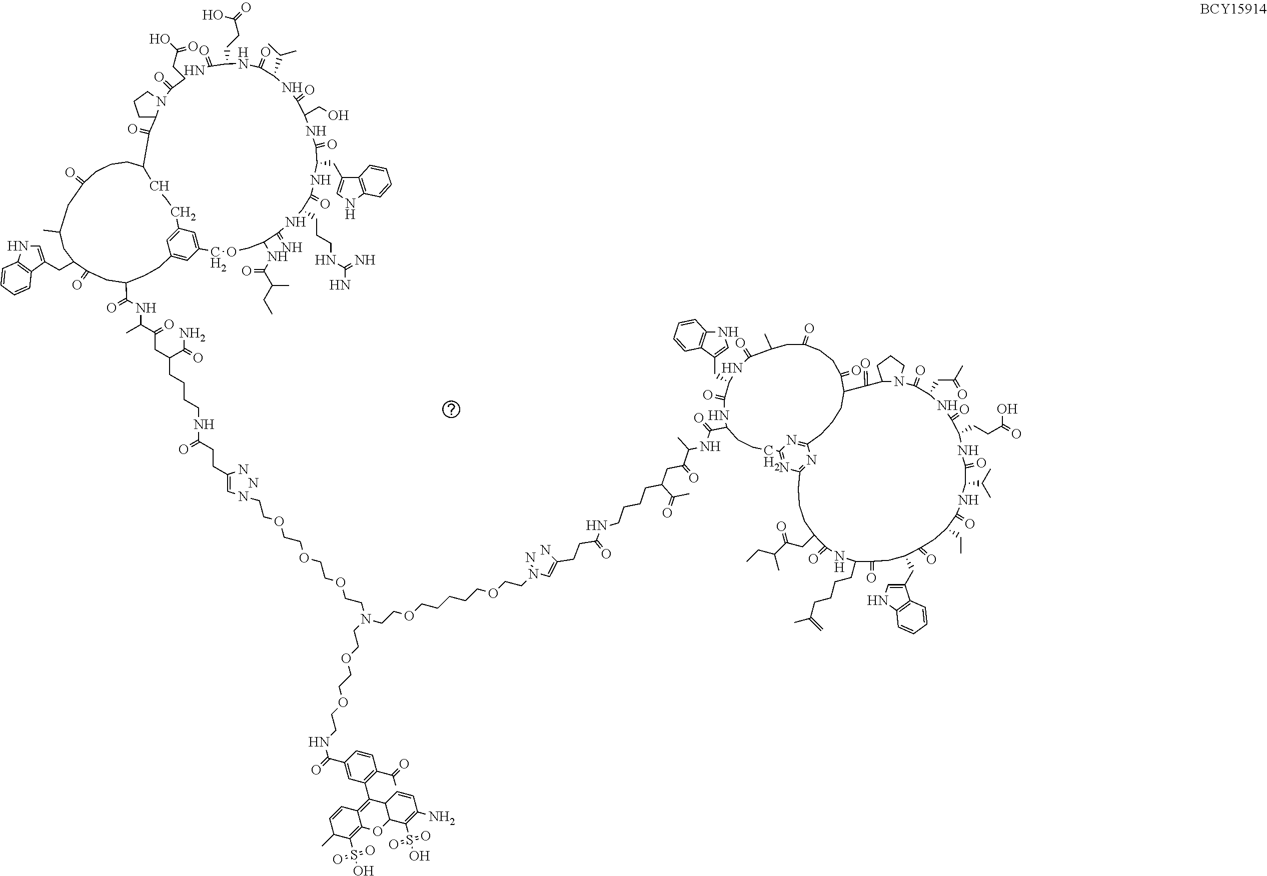

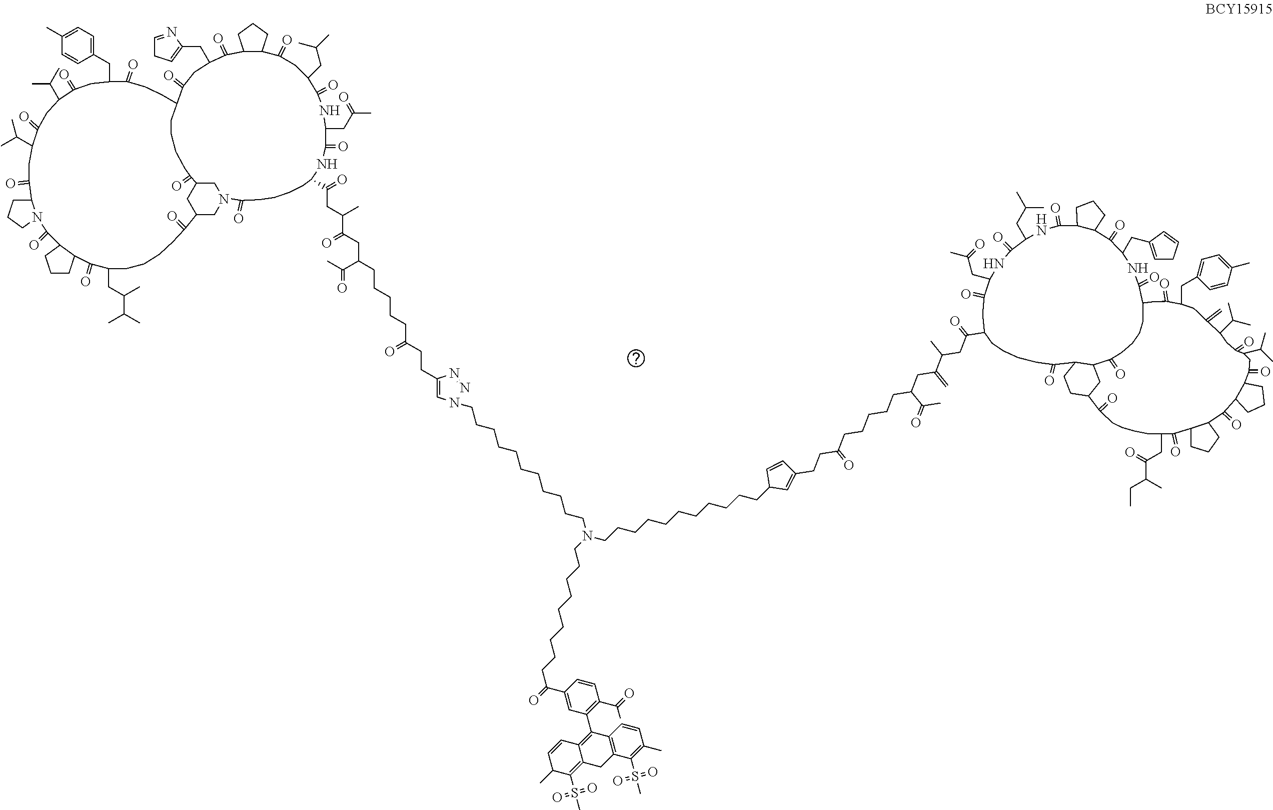

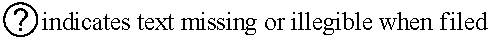

Biological Data

1. NKp46 Fluorescence Polarization (FP) Direct Binding Assay

Affinity of the peptides of the invention for human NKp46 (Acro Biosystems, NC1-H5257 or NC1-H52H4) was determined using a fluorescence polarisation assay. Peptides of the invention were labelled with a fluorescent tag (fluorescein) and diluted to 2.5 nM in 25 mM HEPES with 100 mM NaCl and 0.005% P20, pH 7.4. NKp46 protein was titrated starting at 0.2-5 μM in the same assay buffer as the peptide to assay 1 nM peptide in a total volume of 25 μL in black walled and bottomed low bind low volume 384 well plates. The assay was typically set up by adding 5 μL assay buffer, 10 μL NKp46 protein then 10 μL fluorescent peptide. The concentrations of NKp46 protein were either 2-fold or 3-fold dilutions to give 12 different concentrations starting at 0.2-5 μM. Measurements were conducted on a BMG PHERAstar FS equipped with an FP 485 520 520 optic module at 25° C. with 200 flashes per well and a positioning delay of 0.1 second. Each well was measured every 5 minutes for 60 minutes. The gain used for analysis was determined for each tracer at the end of the 60 minutes where there was no protein in the well. The mP were fit to a standard 1:1 binding model with a quadratic equation to generate a K_{D} value. Selected peptides of the invention were tested in the above-mentioned assay and the results are shown in Table 1.

FP Direct Binding Assay Materials:
Human NKp46 Fc tag (Acro Biosystems, NC1-H5257)
Human NKp46 His tag (Acro Biosystems, NC1-H52H4)

TABLE 1
Fluorescence Polarisation Direct Binding Assay Data
for Selected Bicyclic Peptides of the Invention

| Bicyclic Peptide | Kd (nM) |
|---|---|
| BCY15023 | 1405 |
| BCY15024 | 1042 |
| BCY15025 | 58 |
| BCY15028 | 12 |
| BCY15030 | 106 |
| BCY15035 | 12 |
| BCY15116 | 38 |
| BCY15115 | 440 |
| BCY14652 | 65 |
| BCY15117 | 35 |
| BCY14654 | 1026 |
| BCY15125 | 602 |
| BCY15118 | 647 |
| BCY15119 | 1254 |
| BCY15120 | 1746 |
| BCY15121 | 1741 |
| BCY15122 | 290 |
| BCY15123 | 416 |

2. NKp46 Fluorescence Polarization (FP) Competition Binding Assay

Affinities of peptides of the invention were determined in a fluorescence polarization (FP) competition assay using the fluorescent labelled peptide BCY15035 as the tracer that is competed off by untagged peptides.

The untagged competitor peptides were diluted in assay buffer 25 mM HEPES, 100 mM NaCl, 0.01% P20, pH 7.4. to a top concentration of 5000 nM and diluted 2-fold in 11 steps. The human NKp46-his tagged protein (AcroBiosystems) was diluted to 40 nM final concentration in the assay. Finally, the fluorescent tracer peptide BCY15035 was added at 1 nM. The assay was typically set up by adding 5 μL competitor, 10 μL NKp46 protein then 10 μL fluorescent peptide. The total volume of 25 μL was prepared in black walled and flat-bottomed low binding low volume 384 well plates. Measurements were performed using Envision (PerkinElmer) equipped with the excitation filter FITC FP 480, emission filter FITC FP P-pol 535 and 2^{nd} emission filter FITC FP S-pol 535 and set to 30 flashes. The gain was set in a well containing tracer alone without target protein. The mP-values in the final read at 60 minutes were transformed into percentage inhibition and used to calculate the Ki-values (Kcomp) in the software Sigmaplot (Systat Software Inc) using the equation below:

f=ymax+(ymin−ymax)/Lig*((Lig*((2*((Klig+Kcomp+Lig+Comp−Prot*c)\hat{ }2−3*(Kcomp*(Lig−Prot*c)+Klig*(Comp−Prot*c)+Klig*Kcomp))\hat{ }0.5*COS(ARCCOS((−2*(Klig+Kcomp+Lig+Comp−Prot*c)\hat{ }3+9*(Klig+Kcomp+Lig+Comp−Prot*c)*(Kcomp*(Lig−Prot*c)+Klig*(Comp−Prot*c)+Klig*Kcomp)−27*(−1*Klig*Kcomp*Prot*c))/(2*((((Klig+Kcomp+Lig+Comp−Prot*c)\hat{ }2−3*(Kcomp*(Lig−Prot*c)+Klig*(Comp−Prot*c)+Klig*Kcomp))\hat{ }3)\hat{ }0.5)))/3))−(Klig+Kcomp+Lig+Comp−Prot*c)))/((3*Klig)+((2*((Klig+Kcomp+Lig+Comp−Prot*c)\hat{ }2−3*(Kcomp*(Lig−Prot*c)+Klig*(Comp−Prot*c)+Klig*Kcomp))\hat{ }0.5*COS(ARCCOS((−2*(Klig+Kcomp+Lig+Comp−Prot*c)\hat{ }3+9*(Klig+Kcomp+Lig+Comp−Prot*c)*(Kcomp*(Lig−Prot*c)+Klig*(Comp−Prot*c)+Klig*Kcomp)−27*(−1*Klig*Kcomp*Prot*c))/(2*((((Klig+Kcomp+Lig+Comp−Prot*c)\hat{ }2−3*(Kcomp*(Lig−Prot*c)+Klig*(Comp−Prot*c)+Klig*Kcomp))\hat{ }3)\hat{ }0.5)))/3))−(Klig+Kcomp+Lig+Comp−Prot*c))))

Selected peptides of the invention were tested in the above-mentioned assay and the results are shown in Table 2.

TABLE 2
Competition Fluorescence Polarisation Assay Data
for Selected Bicyclic Peptides of the Invention

| Bicyclic Peptide | Ki (nM) |
|---|---|
| BCY15100 | 49 |
| BCY14455 | 282 |
| BCY15633 | 86 |
| BCY15634 | 98 |
| BCY15635 | 61 |
| BCY15636 | 143 |
| BCY15638 | 255 |
| BCY15639 | 17 |
| BCY15640 | 156 |
| BCY15641 | 129 |
| BCY15642 | 910 |
| BCY15643 | 755 |
| BCY15644 | 159 |
| BCY15645 | >5000 |
| BCY15646 | 107 |
| BCY15648 | 1619 |
| BCY15649 | 391 |
| BCY15650 | 16 |
| BCY15651 | 204 |
| BCY15653 | 411 |
| BCY15654 | >5000 |
| BCY15655 | 324 |
| BCY15656 | 39 |
| BCY15657 | >5000 |
| BCY15658 | >5000 |
| BCY15659 | 226 |
| BCY15660 | >5000 |
| BCY15661 | 1779 |
| BCY15662 | 672 |
| BCY16130 | 321 |
| BCY16131 | >5000 |
| BCY16132 | 8 |
| BCY16133 | 10 |
| BCY16134 | 77 |
| BCY16135 | 639 |
| BCY16136 | 269 |
| BCY16137 | >5000 |
| BCY16138 | >5000 |
| BCY16139 | >5000 |
| BCY16140 | 122 |
| BCY16141 | >5000 |
| BCY16142 | 317 |
| BCY16143 | 464 |
| BCY16144 | 718 |
| BCY16145 | >5000 |

3a. NKp46 SPR Assay (Method 1)

SPR experiments were performed to determine kₐ (M⁻¹s⁻¹), k_{d} (s⁻¹), K_{D} (nM) values (where measurable) of various bicyclic peptides to immobilised NKp46.

For analysis of peptide binding to NKp46, a Biacore 3000 instrument was used with a Cytiva Streptavidin chip. Capture of the protein (recombinant human NKp46-Fc fusion biotinylated via Avi-tag) was carried out at 25° C. with HBS-N (10 mM HEPES, 0.15 M NaCl, pH 7.4) as the running buffer and biotinylated NKp46 was captured to a level of 400-800 RU using a dilution of protein to 0.2 μM in buffer. Buffer was then changed to PBS/0.05% Tween 20 and a dilution series of the peptides was prepared in this buffer with a final DMSO concentration of 0.5% with a top peptide concentration of 100 nM-5000 nM and 6 further 2 or 3-fold dilutions. The SPR analysis was run at 25° C. and a flow rate of 50 μl/min with 60 seconds association and 200-400 seconds dissociation time. Data were corrected for DMSO excluded volume effects. All data were double-referenced for blank injections and reference surface using standard processing procedures and data processing and kinetic fitting were performed using Scrubber software, version 2.0c (BioLogic Software). Data were fitted using simple 1:1 binding model allowing for mass transport effects where appropriate.

Selected peptides of the invention were tested in the above-mentioned assay and the results are shown in Table 3A.

TABLE 3A
SPR Assay Data for Selected Bicyclic Peptides of the Invention

| Bicyclic Peptide | Kd (nM) |
|---|---|
| BCY15013 | 138 |
| BCY15452 | 27 |
| BCY15686 | 276 |
| BCY15687 | 960 |

3b. NKp46 SPR Assay (Method 2)

Peptides were tested against Fc-tagged human NKp46 (ACROBiosystems, cat no. NC1-H5257).

For analysis of peptide binding, a Biacore 8k+ instrument was used with a CM5 chip (Cytiva). All experiments were carried out at 25° C. Anti-human IgG (Fc) antibody (Cytiva) was immobilized on all flow cells by standard amine coupling chemistry. The running buffer was HBS-N(Cytiva, 10 mM HEPES, 0.15 M NaCl, pH 7.4) and the flow rate was 10 μL/min. The carboxymethyl dextran surface was activated with a 1:1 ratio of 0.4 M 1-ethyl-3-(3-dimethylaminopropyl) carbodiimide hydrochloride (EDC)/0.1 M N-hydroxy succinimide (NHS) (Cytiva) for 420s. Anti-human IgG (Fc) antibody was diluted to 12.5 μg/mL in 10 mM sodium acetate pH 5.0 and injected onto the chip surface for 360 s. Residual activated groups were blocked with a 420 s injection of 1 M ethanolamine (pH 8.5). The final surface density was 3000-5000 RU. Fc-tagged human NKp46 was diluted to 0.1 μM in HBS-N and was captured on flow cell 2 only of the sensor chip at a flow rate of 10 μL/min to 500-1000 RU. The buffer was then changed to PBS-P+(Cytiva) supplemented with 2% DMSO. Peptide dilutions were prepared in this buffer to give a final concentration of 2% DMSO. The flow rate was 50 μL/min with 60 s association and 400 s dissociation. Data were corrected for DMSO volume exclusion effects. All data were double-referenced against blank injections and reference surface response using standard processing procedures. Data processing and kinetic fitting was performed using Biacore Insight Evaluation Software with Extended Screening and Characterization Extension (Cytiva). Data were fitted using steady state affinity 1:1 binding model to determine K_{D}. Data were fitted using kinetic 1:1 binding model to determine kinetic constants where appropriate.

Selected peptides of the invention were tested in Method 1 and/or Method 2 and the results are shown in Table 3B.

TABLE 3B
SPR Assay Data for Selected Bicyclic Peptides of the Invention

| Bicyclic Peptide | Kd (nM) | |
|---|---|---|
| BCY15686 | 250 ± 24 | (n = 3) |
| BCY15452 | 13 ± 3 | (n = 2) |
| BCY14455 | 230 | (n = 1) |
| BCY15100 | 36 ± 14 | (n = 4) |
| BCY16133 | 4.9 | (n = 1) |
| BCY17224 | 3.5 ± 1 | (n = 6) |
| BCY14454 | 2760 | (n = 1) |
| BCY14456 | 703 | (n = 1) |
| BCY14457 | 2270 | (n = 1) |
| BCY14458 | 1260 | (n = 1) |
| BCY15013 | 59 | (n = 1) |
| BCY15098 | 1070 | (n = 2) |
| BCY15099 | 1117 ± 681 | (n = 7) |
| BCY15102 | 1090 ± 99 | (n = 2) |
| BCY15103 | 1665 ± 544 | (n = 2) |
| BCY15104 | 2880 | (n = 1) |
| BCY15105 | 3730 | (n = 1) |
| BCY15106 | 2670 | (n = 1) |
| BCY15107 | 396 ± 114 | (n = 9) |
| BCY15108 | 642 | (n = 1) |
| BCY15109 | 274 | (n = 1) |
| BCY15568 | 69 | (n = 1) |
| BCY15637 | 131 | (n = 1) |
| BCY15639 | 11 ± 8 | (n = 2) |
| BCY15647 | 80 | (n = 1) |
| BCY15650 | 10 ± 3 | (n = 2) |
| BCY15652 | 44 | (n = 1) |
| BCY15656 | 34 ± 3 | (n = 2) |
| BCY15687 | 783 ± 167 | (n = 3) |
| BCY16132 | 3.9 | (n = 1) |
| BCY16834 | 83 | (n = 1) |
| BCY16837 | 42 | (n = 1) |
| BCY16840 | 60 | (n = 1) |
| BCY16842 | 2470 | (n = 1) |
| BCY16843 | 3010 | (n = 1) |
| BCY17662 | 8.1 | (n = 1) |
| BCY18004 | 414 | (n = 1) |
| BCY18005 | 729 | (n = 1) |
| BCY18260 | 1240 | (n = 1) |
| BCY18261 | 543 | (n = 1) |
| BCY18262 | 3180 | (n = 1) |
| BCY18263 | 1740 | (n = 1) |
| BCY18265 | 1480 | (n = 1) |
| BCY18266 | >10000 | (n = 1) |
| BCY18269 | 1640 | (n = 1) |
| BCY18272 | 3190 | (n = 1) |
| BCY18273 | 326 | (n = 1) |
| BCY18277 | 1520 | (n = 1) |
| BCY18278 | 335 | (n = 1) |
| BCY18279 | 599 | (n = 1) |
| BCY18280 | 1270 | (n = 1) |
| BCY18281 | 424 | (n = 1) |
| BCY18282 | 661 | (n = 1) |
| BCY18285 | 3760 | (n = 1) |
| BCY18286 | 1230 | (n = 1) |
| BCY18287 | 637 | (n = 1) |
| BCY18288 | 354 | (n = 1) |
| BCY18289 | 572 | (n = 1) |
| BCY18290 | 4890 | (n = 1) |
| BCY18291 | 2850 | (n = 1) |
| BCY18292 | 1460 | (n = 1) |
| BCY18293 | 3380 | (n = 1) |
| BCY18294 | 3260 | (n = 1) |
| BCY18295 | 6410 | (n = 1) |
| BCY18299 | 1800 | (n = 1) |
| BCY18301 | 4030 | (n = 1) |
| BCY18302 | 5380 | (n = 1) |
| BCY18303 | 2750 | (n = 1) |
| BCY18304 | 1530 | (n = 1) |
| BCY18305 | 907 | (n = 1) |
| BCY18307 | 2110 | (n = 1) |
| BCY18309 | 2570 | (n = 1) |
| BCY18310 | 802 | (n = 1) |
| BCY18311 | 795 | (n = 1) |
| BCY18312 | 1810 | (n = 1) |
| BCY18313 | 2750 | (n = 1) |
| BCY18314 | 3150 | (n = 1) |
| BCY18315 | 878 | (n = 1) |
| BCY18316 | 1510 | (n = 1) |
| BCY18317 | 2080 | (n = 1) |
| BCY18318 | 1920 | (n = 1) |
| BCY18319 | 991 | (n = 1) |
| BCY18320 | 622 | (n = 1) |
| BCY18321 | 548 | (n = 1) |
| BCY18322 | 466 | (n = 1) |
| BCY18323 | 2340 | (n = 1) |
| BCY18324 | 1500 | (n = 1) |
| BCY18325 | 854 | (n = 1) |
| BCY18326 | 2780 | (n = 1) |
| BCY18327 | 1670 | (n = 1) |
| BCY18328 | 887 | (n = 1) |
| BCY18433 | 36 | (n = 1) |
| BCY18434 | 44 | (n = 1) |
| BCY18435 | 91 | (n = 1) |
| BCY18437 | 47 | (n = 1) |
| BCY18439 | 36 | (n = 1) |
| BCY18440 | 149 | (n = 1) |
| BCY18441 | 7.7 | (n = 1) |
| BCY18442 | 7.1 | (n = 1) |
| BCY18443 | 6.3 | (n = 1) |
| BCY18444 | 3.9 | (n = 1) |
| BCY18470 | 22 | (n = 1) |
| BCY19682 | 9 | (n = 1) |
| BCY19683 | 10 | (n = 1) |
| BCY19684 | 9.4 | (n = 1) |
| BCY19685 | 10 | (n = 1) |
| BCY19686 | 44 | (n = 1) |
| BCY19687 | 28 | (n = 1) |
| BCY19688 | >500 | (n = 1) |
| BCY19689 | 16 | (n = 1) |
| BCY19690 | 19 | (n = 1) |
| BCY19691 | >500 | (n = 1) |
| BCY19692 | >500 | (n = 1) |
| BCY19693 | 17 | (n = 1) |
| BCY19694 | 2510 | (n = 1) |
| BCY19695 | 2380 | (n = 1) |
| BCY19696 | >10000 | (n = 1) |
| BCY19697 | 3730 | (n = 1) |
| BCY19698 | >10000 | (n = 1) |
| BCY19699 | 2610 | (n = 1) |
| BCY19700 | >10000 | (n = 1) |
| BCY19701 | 4030 | (n = 1) |
| BCY19702 | 2680 | (n = 1) |
| BCY19703 | 1690 | (n = 1) |
| BCY19704 | 1620 | (n = 1) |
| BCY19706 | 1200 | (n = 1) |
| BCY19707 | 1050 | (n = 1) |
| BCY19708 | 1740 | (n = 1) |
| BCY19710 | 2360 | (n = 1) |
| BCY19711 | 2750 | (n = 1) |
| BCY19712 | 3140 | (n = 1) |
| BCY19713 | 1830 | (n = 1) |
| BCY19717 | 598 | (n = 1) |
| BCY19718 | 1110 | (n = 1) |
| BCY19719 | 323 | (n = 1) |
| BCY19720 | 3750 | (n = 1) |
| BCY19721 | 374 | (n = 1) |
| BCY19722 | 1460 | (n = 1) |
| BCY19723 | 2270 | (n = 1) |
| BCY19724 | 1390 | (n = 1) |
| BCY19725 | 457 | (n = 1) |
| BCY19730 | >10000 | (n = 1) |
| BCY19732 | 964 | (n = 1) |
| BCY19733 | 354 | (n = 1) |
| BCY19734 | 611 | (n = 1) |
| BCY19735 | 460 | (n = 1) |

4. Binding to CHO-K1/NKp46 Cell Line

The affinity of NKp46 dimeric bicyclic peptides conjugated to Alexa Fluor® 488 to commercially available NKp46 overexpressing CHO-K1 cells was determined using a flow cytometry assay.

Commercially available CHO-K1 cells stably transfected with human activating natural cell receptor 46 (NKp46, also known as natural cytotoxicity triggering receptor 1 (NCR1) or CD355), herein referred to as CHO-K1/NKp46 cells, were procured from Abeomics. CHO-K1/NKp46 cells were grown in DMEM medium (Corning® 15-017 CV) supplemented with 10% heat-inactivated fetal bovine serum (FBS; Corning® 35-011-CV), 10 mM HEPES (Gibco™ 15-630-080), 1% Penicillin Streptomycin (Corning™ 30-002-CI), and 10 μg/mL of Blasticidin (Gibco™ A1113902). Culture maintenance and passage conditions were followed according to manufacturer recommendations.

On the day of the experiment, CHO-K1/NKp46 were detached from the culture vessel with Trypsin/EDTA, washed once at 500 rpm for 5 minutes in 15 mL of prewarmed working medium defined as: DMEM medium (Corning® 15-017 CV) supplemented with 10% heat-inactivated fetal bovine serum (FBS; Corning® 35-011-CV), 10 mM HEPES (Gibco™ 15-630-080), and 1% Penicillin Streptomycin (Corning™ 30-002-Cl). Cell pellet was then resuspended in working medium at a concentration of 3×10⁵ cells/mL. Subsequently, 100 μL of cell suspension was plated in a 96-well V-bottom polypropylene plate (Greiner Bio-One 651201).

NKp46 dimeric bicyclic peptides conjugated to Alexa Fluor® 488 were diluted in working medium and were added to the plate at a starting concentration of 1 μM and titrated in a 1:4 dilution series to perform an 11-point serial dilution. Plates were then incubated for 1 hour at room temperature in the dark. Post-incubation, the plates were centrifuged at 500 rpm for 5 minutes and supernatant discarded. Samples were then washed twice in 200 μL of 1× phosphate buffer saline (PBS; Gibco™ 10-010-023) at 500 rpm for 5 minutes. Cells were resuspended in 100 μL of PBS and transferred to a new 96-well V-bottom polypropylene plate (Greiner Bio-One 651201). Plates were centrifuged at 500 rpm for 5 minutes and supernatant discarded.

Preparation of samples for flow cytometry: Zombie Violet™ Fixable Viability Dye (BioLegend® 423113) was prepared as a 1:1000 dilution in PBS and 100 μL of viability dye was added to each well and incubated in the dark at 4° C. for 30 minutes. To ensure NKp46 expression, untreated CHO-K1/NKp46 cells were stained with either anti-human NKp46 or an isotype control. Antibody cocktails were prepared by diluting 1.5 μL of either, PE anti-human NKp46 (BioLegend® 331908; clone 9E2) or PE isotype control IgG1, K (BioLegend® 400112), per 100 μL of stain buffer (1×PBS supplemented with 2% FBS). Cells were resuspended in master mix cocktail (100 μL) and incubated at 4° C. for 30 minutes in the dark. Subsequently, cells were washed 3 times in 100 μL of stain buffer for 5 minutes at 500 rpm and supernatant was discarded. Cells resuspended in 200 μL of stain buffer were kept at 4° C. and in the dark until read by BD FACSCelesta™ flow cytometer.

Flow cytometry results were analyzed in FlowJo™ software with a gating strategy to exclude debris, gate on single and live cells only, and determining the geometric mean of the Alexa Fluor® 488+/−populations. The binding affinity of the bicyclic peptides were calculated using a four-parameter logistic regression using Prism GraphPad software.

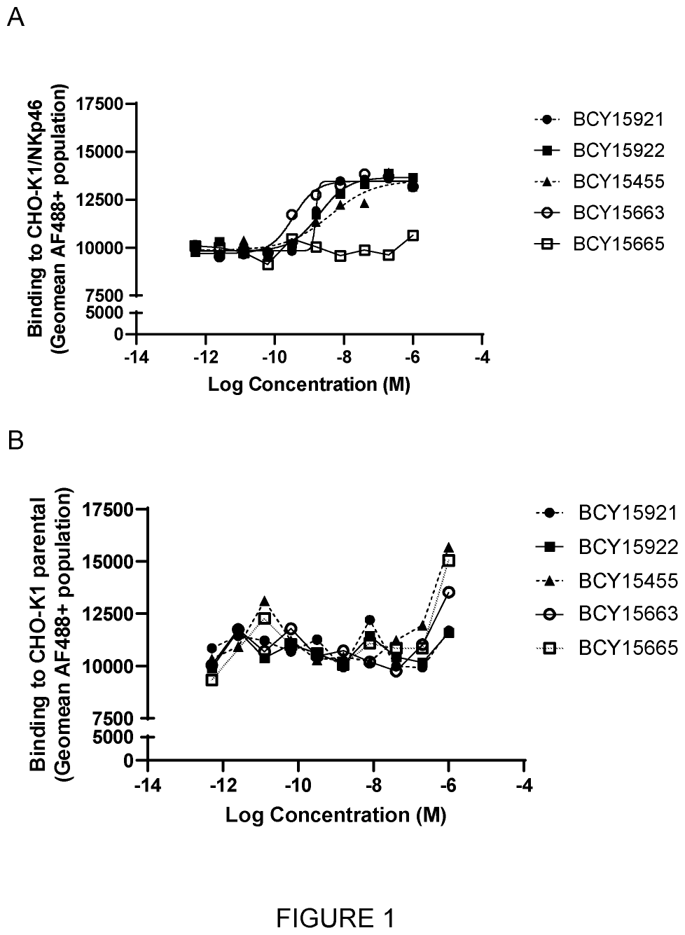

The data shown in FIG. 1 and Table 4 illustrates that Alexa Fluor® 488-tagged NKp46 bicyclic peptide dimers bind in a dose-dependent manner to NKp46 expressing cells.

TABLE 4
Binding affinities (K_{d, app}) for the Alexa Fluor ® 488
NKp46 dimer bicyclic peptides binding to CHO-K1/NKp46 cells

| Alexa Fluor ® | Binding Affinity |
|---|---|
| 488 NKp46 Dimer | to CHO-K1/NKp46 |
| Bicyclic Peptide | population K_{d, app} (nM) |
| BCY15921 | 1.48 |
| BCY15922 | 1.48 |
| BCY15455 | 2.25 |
| BCY15663 | 0.30 |

5. Binding to PBMCs

The affinity of NKp46 dimeric bicyclic peptides conjugated to Alexa Fluor® 488 to primary healthy peripheral blood mononuclear cells (PBMCs) was evaluated using a flow cytometry assay.

On the day of the experiment, medium was prepared by supplementing RPMI-1640 (Gibco™ 11875-093; with L-glutamine) with 10% heat-inactivated fetal bovine serum (FBS; Corning® 35-011-CV), 10 mM HEPES (Gibco™ 15-630-080), and 1% Penicillin Streptomycin (Corning™ 30-002-Cl), herein referred to as working medium. Previously isolated human peripheral blood mononuclear cells (PBMCs), from whole blood, were quick thawed in a water bath and washed once at 500 rpm for 5 minutes in 10 mL of prewarmed working medium. PBMC pellet was then resuspended to concentration of 5×10⁶ cells/mL in working medium and rested overnight (12-18 hours) horizontally in a tissue-culture coated flask (T-183; CELLTREAT Scientific 229351).

Post-overnight incubation, PBMCs were removed from the flask and washed once at 500 rpm for 5 minutes in 10 mL of prewarmed working medium. PBMC pellet was then resuspended in working medium at a concentration of 5×10⁵ cells/mL. Subsequently, 100 μL of cell suspension was plated in a 96-well V-bottom polypropylene plate (Greiner Bio-One 651201). Bicyclic peptides were diluted in working medium and added to the corresponding cell plate at a starting concentration of 300 nM titrated in a 1:4 dilution series to perform a 12-point serial dilution. Plates were then incubated 1 hour at 37° C., 5% CO₂. Post-incubation, plate was centrifuged at 500 rpm for 5 minutes and supernatant discarded. Samples were then washed once in 200 μL of 1× phosphate buffer saline (PBS; Gibco™ 10-010-023) at 500 rpm for 5 minutes and supernatant was discarded.

Preparation of samples for flow cytometry: Zombie Aqua™ Fixable Viability Dye (BioLegend® 423102) was prepared as a 1:1000 dilution in PBS and 100 μL of viability dye was added to each well and incubated in the dark at 4° C. for 30 minutes. Subsequently, wells were washed with 100 μL of PBS for 5 minutes at 500 rpm and supernatant was discarded. Next, human TruStain FcX™ block (BioLegend® 422302) was prepared by diluting 1.5 μL of FcX in 25 μL of stain buffer (1×PBS supplemented with 2% FBS). Fc block solution (25 μL/well) was incubated at room temperature (RT) for 10 minutes in the dark. Antibody master mix cocktail was prepared by diluting 1.5 μL the following antibodies per 100 μL of stain buffer: Brilliant Violet 605™ anti-human CD3 (BioLegend® 344836; clone SK7), and PE/Cyanine7 anti-human CD56 (BioLegend® 362510; clone 5.1H11). Cells were resuspended in master mix cocktail (100 μL) and incubated at 4° C. for 30 minutes in the dark. Subsequently, cells were washed 3 times in 100 μL of stain buffer for 5 minutes at 500 rpm and supernatant was discarded. Cells were resuspended in 200 μL of stain buffer and kept at 4° C. in the dark until read by BD FACSCelesta™ flow cytometer.

Flow cytometry results were analyzed in FlowJo™ software with a gating strategy to evaluate the lymphocyte population then exclude debris, gate on single and live cells only, and determining the geometric mean of the Alexa Fluor® 488 in the CD3+ and CD3−/CD56+ populations. The binding affinities were calculated using a four-parameter logistic regression using Prism GraphPad™ 8.0.2 software.

TABLE 5
Binding affinities (K_{d, app}) for the Alexa Fluor ® 488
NKp46 dimers binding to sub-populations in PBMC cells

| | Binding Affinity to |
|---|---|
| Alexa Fluor ® | Total CD56 |
| 488 NKp46 Dimer | population K_{d, app} |
| Bicyclic Peptide | (nM) |
| BCY15663 | 0.028 ± 0.006 |

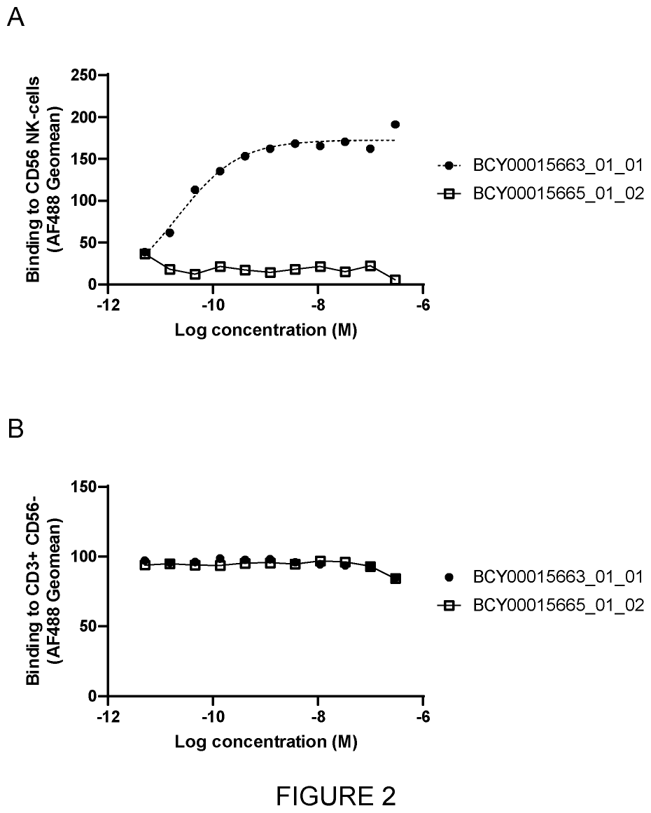

The data shown in Table 5 and FIG. 2 demonstrates that Alexa Fluor® 488-tagged NKp46 BCY15663 elicits a dose-dependent binding to NK cells only (CD56+ populations). Comparatively, non-binding Alexa Fluor® 488-tagged NKp46 BCY15665 elicits no binding in any PBMC population.

6a. FcγRIII (CD16a) SPR Experimental Description (Method A)

SPR experiments were performed to determine kₐ (M⁻¹s⁻¹), k_{d} (s⁻¹), K_{D} (nM) values (where measurable) of various bicyclic peptides to immobilized human FcγRIII proteins.

For analysis of peptide binding, a Biacore 3000 or T200 instrument was used with a CM5 chip (GE Healthcare). Streptavidin was immobilized on the chip using standard amine-coupling chemistry at 25° C. with HBS-N (10 mM HEPES, 0.15 M NaCl, pH 7.4) as the running buffer. Briefly, the carboxymethyl dextran surface was activated with a 7 min injection of a 1:1 ratio of 0.4 M 1-ethyl-3-(3-dimethylaminopropyl) carbodiimide hydrochloride (EDC)/0.1 M N-hydroxy succinimide (NHS) at a flow rate of 10 μl/min. For capture of streptavidin, the protein was diluted to 0.2 mg/ml in 10 mM sodium acetate (pH 4.5) and captured by injecting 120 μl of onto the activated chip surface. Residual activated groups were blocked with a 7 min injection of 1 M ethanolamine (pH 8.5) and biotinylated FcγRIII captured to a level of 400-1,400 RU. Buffer was changed to PBS/0.05% Tween 20 and a dilution series of the peptides was prepared in this buffer with a final DMSO concentration of 0.5%. The top peptide concentration ranged from 200 nM-20 μM (depending on expected affinity) with 6 further 2- or 3-fold dilutions. The SPR analysis was run at 25° C. at a flow rate of 50 μl/min with 60 seconds association and dissociation between 60 and 200 seconds. Data were corrected for DMSO excluded volume effects. All data were double-referenced for blank injections and reference surface using standard processing procedures and data processing and kinetic fitting were performed using Scrubber software, version 2.0c (BioLogic Software). Data were fitted using simple 1:1 binding model allowing for mass transport effects where appropriate.

Selected peptides of the invention were tested in the above-mentioned assay and the results are shown in Table 6A:

TABLE 6A
SPR Assay Data for Selected Bicyclic Peptides of the Invention

| Bicyclic Peptide | Kd (nM) |
|---|---|
| BCY13886 | 2200 |
| BCY13883 | 1800 |

6b. FcγRIII (CD16a) SPR Experimental Description (Method B)

For analysis of peptide binding, a Biacore 8k+ instrument was used with a CM5 chip (Cytiva). All experiments were carried out at 25° C. Streptavidin was immobilized on all flow cells by standard amine coupling chemistry. The running buffer was HBS-N(Cytiva, 10 mM HEPES, 0.15 M NaCl, pH 7.4) and the flow rate was 10 μl/min. The carboxymethyl dextran surface was activated with a 1:1 ratio of 0.4 M 1-ethyl-3-(3-dimethylaminopropyl) carbodiimide hydrochloride (EDC)/0.1 M N-hydroxy succinimide (NHS) (Cytiva) for 420s. Streptavidin (ProSpec, cat no. PRO-791) was diluted to 0.2 mg/ml in 10 mM sodium acetate pH 4.5 and was injected onto the chip surface for 600 s. Residual activated groups were blocked with a 420 s injection of 1 M ethanolamine (pH 8.5). The final surface density was 2000-3000 RU. Biotinylated CD16a was diluted to 5 μg/ml in HBS-N and was captured on flow cell 2 only of the streptavidin chip at a flow rate of 10 μl/min to 1500-2500 RU. The buffer was then changed to PBS-P+(Cytiva) supplemented with 0.5-2% DMSO. Peptide dilutions were prepared in this buffer to give a final concentration of 0.5-2% DMSO. The flow rate was 30 μl/min with 100 s association and 400 s dissociation. Data were corrected for DMSO volume exclusion effects. All data were double-referenced against blank injections and reference surface response using standard processing procedures. Data processing and kinetic fitting was performed using Biacore Insight Evaluation Software with Extended Screening and Characterization Extension (Cytiva). Data were fitted using steady state affinity 1:1 binding model to determine K_{D}. Data were fitted using kinetic 1:1 binding model to determine kinetic constants where appropriate.

Selected peptides of the invention were tested in Method A and/or Method B and the results are shown in Table 6B:

TABLE 6B
SPR Assay Data for Selected Bicyclic Peptides of the Invention

| | Kd (nM) | | Kd (nM) | |
|---|---|---|---|---|
| Bicyclic Peptide | (F Variant) | | (V Variant) | |
| BCY11673 | 17130 | (n = 1) | 3306 | (n = 1) |
| BCY11808 | 6800 | (n = 1) | 3710 | (n = 1) |
| BCY11809 | 28560 | (n = 1) | 4326 | (n = 1) |
| BCY11810 | 3611 | (n = 1) | 831.5 | (n = 1) |
| BCY11811 | 8237 | (n = 1) | 1226 | (n = 1) |
| BCY11812 | 11800 | (n = 1) | 2404 | (n = 1) |
| BCY12621 | 3600 | (n = 1) | 1900 | (n = 1) |
| BCY12622 | 1000 | (n = 1) | 4000 | (n = 1) |
| BCY13883 | 1759 ± 496 | (n = 7) | 1518 ± 677 | (n = 7) |
| BCY13886 | 8358 ± 5291 | (n = 4) | 4285 ± 1743 | (n = 4) |
| BCY14950 | 1992 ± 534 | (n = 5) | 1840 ± 1197 | (n = 5) |
| BCY14953 | 2980 | (n = 1) | 2440 | (n = 1) |
| BCY14954 | 6160 | (n = 1) | 4350 | (n = 1) |
| BCY14955 | 1520 | (n = 1) | 440 | (n = 1) |
| BCY14956 | 2660 | (n = 1) | 2150 | (n = 1) |
| BCY15044 | 15460 | (n = 1) | 39620 | (n = 1) |
| BCY15045 | 21940 | (n = 1) | 39650 | (n = 1) |
| BCY15685 | 7373 ± 3880 | (n = 7) | 1846 ± 377 | (n = 6) |
| BCY16165 | 3592 ± 944 | (n = 5) | 3230 | (n = 1) |
| BCY16493 | 4400 | (n = 1) | 7330 | (n = 1) |
| BCY16494 | 2870 | (n = 1) | 2820 | (n = 1) |
| BCY16495 | 3900 | (n = 1) | 5150 | (n = 1) |
| BCY16498 | 2360 | (n = 1) | 2810 | (n = 1) |
| BCY16499 | 8750 | (n = 1) | 9590 | (n = 1) |
| BCY16501 | 7960 ± 948 | (n = 2) | 3225 ± 2510 | (n = 2) |
| BCY16502 | 3090 | (n = 1) | 4910 | (n = 1) |
| BCY16504 | >5000 | (n = 1) | >5000 | (n = 1) |
| BCY16505 | >5000 | (n = 1) | >5000 | (n = 1) |
| BCY16506 | >5000 | (n = 1) | >5000 | (n = 1) |
| BCY16507 | >5000 | (n = 1) | >5000 | (n = 1) |
| BCY16508 | 2980 | (n = 1) | 4590 | (n = 1) |
| BCY16509 | 2820 | (n = 1) | 4990 | (n = 1) |
| BCY16510 | 3180 | (n = 1) | >5000 | (n = 1) |
| BCY16511 | 4020 | (n = 1) | 5000 | (n = 1) |
| BCY16512 | 4200 | (n = 1) | 4390 | (n = 1) |
| BCY16520 | >5000 | (n = 1) | >5000 | (n = 1) |
| BCY16522 | 2560 | (n = 1) | 4400 | (n = 1) |
| BCY16523 | 2600 | (n = 1) | 4870 | (n = 1) |
| BCY16524 | 3890 | (n = 1) | >5000 | (n = 1) |
| BCY16527 | 3970 | (n = 1) | >5000 | (n = 1) |
| BCY16528 | 3060 | (n = 1) | >5000 | (n = 1) |
| BCY16529 | >5000 | (n = 1) | >5000 | (n = 1) |
| BCY16599 | 1880 | (n = 1) | 386 | (n = 1) |
| BCY16608 | 2890 | (n = 1) | 516 | (n = 1) |
| BCY16610 | 896 ± 631 | (n = 2) | 614 ± 395 | (n = 2) |
| BCY19032 | 10500 | (n = 1) | 4640 | (n = 1) |
| BCY19033 | 1017 ± 487 | (n = 2) | 421 ± 133 | (n = 2) |
| BCY19034 | 1810 | (n = 1) | 1080 | (n = 1) |
| BCY19035 | 4000 | (n = 1) | 1110 | (n = 1) |
| BCY19036 | 618 ± 249 | (n = 4) | 382 ± 117 | (n = 4) |
| BCY19037 | 1070 | (n = 1) | 737 | (n = 1) |
| BCY19038 | >10000 | (n = 1) | 6850 | (n = 1) |
| BCY19039 | 772 | (n = 1) | 281 | (n = 1) |
| BCY19040 | 1600 | (n = 1) | 1240 | (n = 1) |
| BCY19041 | 1820 | (n = 1) | 1310 | (n = 1) |
| BCY19042 | 4270 | (n = 1) | 1370 | (n = 1) |
| BCY19043 | 797 | (n = 1) | 731 | (n = 1) |
| BCY19044 | 1670 | (n = 1) | 858 | (n = 1) |
| BCY19045 | 1260 | (n = 1) | 744 | (n = 1) |
| BCY19046 | 1600 | (n = 1) | 902 | (n = 1) |
| BCY19048 | 3290 | (n = 1) | 5690 | (n = 1) |
| BCY19049 | 1300 | (n = 1) | 1140 | (n = 1) |
| BCY19050 | 2150 | (n = 1) | 2850 | (n = 1) |
| BCY19053 | 2720 | (n = 1) | 2620 | (n = 1) |
| BCY19055 | 5880 | (n = 1) | 6960 | (n = 1) |
| BCY19057 | 7650 | (n = 1) | 6110 | (n = 1) |
| BCY19060 | 5880 | (n = 1) | 3090 | (n = 1) |
| BCY19061 | 3730 | (n = 1) | 6320 | (n = 1) |
| BCY19063 | >10000 | (n = 1) | 25400 | (n = 1) |
| BCY19064 | 2440 | (n = 1) | 2960 | (n = 1) |
| BCY19065 | 2900 | (n = 1) | 1320 | (n = 1) |
| BCY19068 | >10000 | (n = 1) | >10000 | (n = 1) |
| BCY19416 | >10000 | (n = 1) | >10000 | (n = 1) |
| BCY19417 | >10000 | (n = 1) | >10000 | (n = 1) |
| BCY19596 | 1710 | (n = 1) | 837 | (n = 1) |
| BCY20360 | 998 | (n = 1) | 496 | (n = 1) |
| BCY20361 | 553 | (n = 1) | 401 | (n = 1) |
| BCY20363 | >10000 | (n = 1) | >10000 | (n = 1) |
| BCY20364 | 4151 | (n = 1) | 1371 | (n = 1) |
| BCY20365 | 6790 | (n = 1) | 3486 | (n = 1) |
| BCY20366 | 706 | (n = 1) | 244 | (n = 1) |
| BCY20367 | 1673 | (n = 1) | 473 | (n = 1) |
| BCY20368 | 1428 | (n = 1) | 531 | (n = 1) |
| BCY20369 | 910 | (n = 1) | 415 | (n = 1) |
| BCY20370 | 1267 | (n = 1) | 639 | (n = 1) |
| BCY20371 | 2151 | (n = 1) | 1795 | (n = 1) |
| BCY20372 | 2414 | (n = 1) | 869 | (n = 1) |
| BCY20373 | 2696 | (n = 1) | 1017 | (n = 1) |
| BCY20374 | 958 | (n = 1) | 799 | (n = 1) |
| BCY20375 | 4040 | (n = 1) | 2156 | (n = 1) |
| BCY20376 | 2875 | (n = 1) | 1542 | (n = 1) |
| BCY20377 | 5171 | (n = 1) | 3566 | (n = 1) |
| BCY20378 | 5206 | (n = 1) | 1978 | (n = 1) |
| BCY20379 | 4319 | (n = 1) | 1430 | (n = 1) |
| BCY20380 | 426 | (n = 1) | 152 | (n = 1) |
| BCY20382 | 385 | (n = 1) | 158 | (n = 1) |
| BCY20383 | 470 | (n = 1) | 202 | (n = 1) |
| BCY20384 | 263 | (n = 1) | 129 | (n = 1) |
| BCY20385 | 103 | (n = 1) | 36 | (n = 1) |
| BCY20386 | 352 | (n = 1) | 132 | (n = 1) |
| BCY20388 | 2983 | (n = 1) | 1075 | (n = 1) |
| BCY20389 | 459 | (n = 1) | 250 | (n = 1) |
| BCY20390 | >10000 | (n = 1) | 6652 | (n = 1) |
| BCY20392 | 825 | (n = 1) | 385 | (n = 1) |
| BCY20394 | 2376 | (n = 1) | 1001 | (n = 1) |
| BCY20395 | 3397 | (n = 1) | 2136 | (n = 1) |
| BCY20396 | 1659 | (n = 1) | 537 | (n = 1) |
| BCY20397 | 753 | (n = 1) | 430 | (n = 1) |
| BCY20526 | 371 | (n = 1) | 157 | (n = 1) |
| BCY20527 | 348 | (n = 1) | 147 | (n = 1) |
| BCY20528 | 395 | (n = 1) | 176 | (n = 1) |
| BCY20529 | 558 | (n = 1) | 249 | (n = 1) |

7. IgG Competition Assay

Bicyclic peptides were evaluated with a CD16a-IgG competition assay using Perkin Elmer AlphaScreen™ technology. Acceptor beads (Perkin Elmer 6762003) were chemically conjugated via a reductive amination reaction with a mouse IgG2a mAb to link via the free amines, based the manufacturer's protocol. Briefly, beads were centrifuged and resuspended in a 130 mM sodium phosphate buffered solution pH 8.0 containing 0.2 mg mAb, 20 mM NaBH₃CN (Sigma 296945) and 0.06% P20 surfactant (Cytiva Life Sciences, BR-1000-54) and incubated on a rotary shaker at 44° C. for 22 hours. Unbound sites were then blocked by adding a final concentration of 3 mg/ml carboxymethoxylamine (CMO) (Sigma C13408), before continuing shaking incubation for a further 1 hour. Beads were washed by centrifugation and resuspension in 100 mM TRIS-HCl pH 8.0 (Sigma T2694), before a final resuspension in PBS (GIBCO 14190)+0.05% Proclin-300 (Sigma 48912-U), at 5 mg/ml and storage at 4° C. until use.

The assay was performed in white 384 well plates (PerkinElmer, 6007290) using assay buffer 25 mM HEPES (Sigma-Aldrich, H0887), 100 mM NaCl (Sigma-Aldrich, S5150), 0.5% BSA (Sigma-Aldrich, A3294), 0.05% P20 (Cytiva Life Sciences, BR-1000-54), pH7.4 for all dilutions. Bicyclic peptides were titrated and incubated with 10 nM final concentration of C-terminally biotinylated human CD16a F176 (ACRO Biosystems CDA-H82E8) or N-terminally biotinylated V176 variant (R&D Systems 4325-Fc, biotinylated in-house) and incubated at room temperature for 30 minutes. Mouse IgG2a-conjugated acceptor beads described above were added at a final concentration of 20 ug/ml and incubated at room temperature for 30 minutes under subdued lighting. Streptavidin coated donor beads (PerkinElmer, 6760002) were added a final concentration of 20 ug/ml and incubated at room temperature for 60 minutes under subdued lighting. Luminescence upon excitation at 570 nM was read using a BMG Labtech Pherastar FS plate-reader. Data was normalised to a high control containing all assay components except Bicyclic peptide and fit to a four-parameter non-linear regression in GraphPad Prism 8.0.2 to generate an IC50 value.

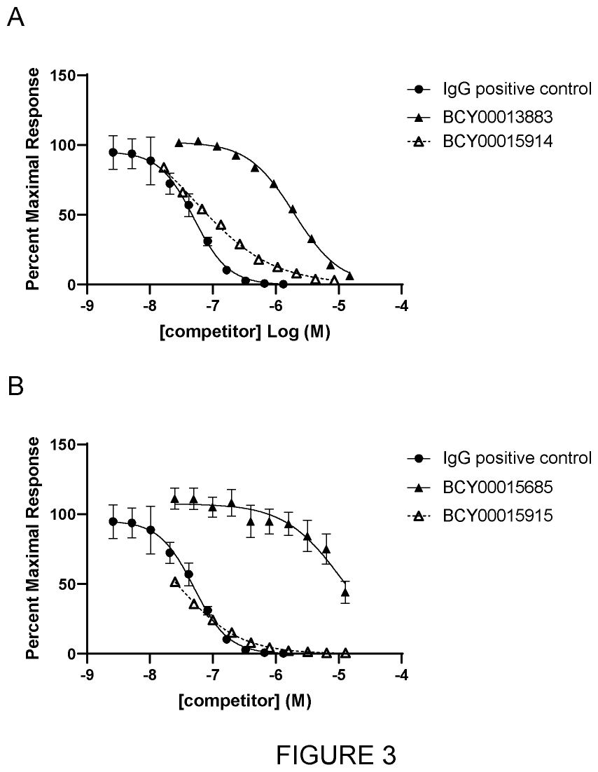

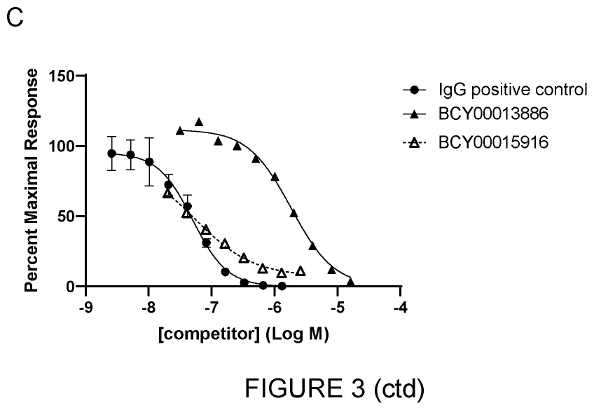

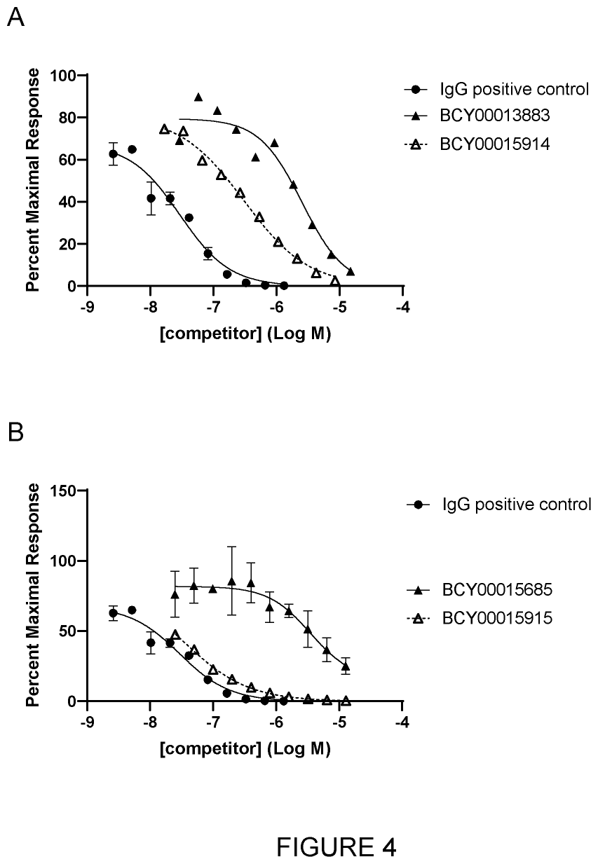

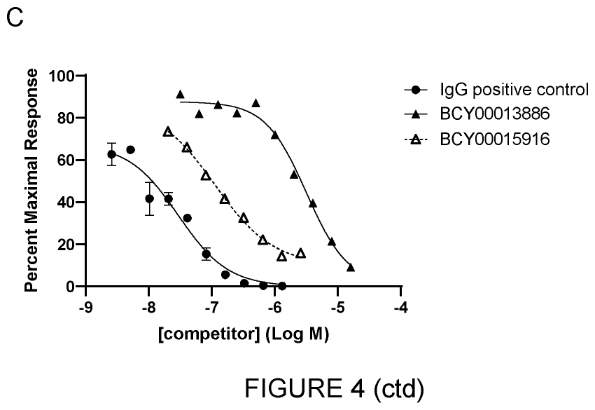

Selected peptides of the invention were tested in the above-mentioned assay and the results are shown in Table 7 and FIGS. 3 and 4. The data in Table 7 and FIGS. 3 and 4 show that the peptides of the invention bind to a biologically relevant epitope on FcγRIIIA (F and V-variants) and are able to compete with IgG for binding to this epitope.

TABLE 7
IgG Competition Assay Data for Selected
Bicyclic Peptides of the Invention

| Bicyclic | IC50 (nM) | IC50 (nM) |
|---|---|---|
| Peptide/Dimer | (F Variant) | (V Variant) |
| BCY15685 | 11000 | 3480 |
| BCY13886 | 1830 | 3190 |
| BCY13883 | 1950 | 2540 |
| BCY15914 | 51.1 | 308 |
| BCY15915 | 27.8 | 32.5 |
| BCY15916 | 52.6 | 113 |

8. CD16a/FCgRIIIa HTRF Assay

Binding to cellular CD16a/FCgRIIIa (F158 variant) was determined through competition with IgG for binding to CD16a receptors expressed on HEK293 cells. This assay was based on fluorescence resonance energy transfer (FRET) whereby a flash of light will induce FRET between the CD16a Terbium donor dye from the IgG-d2 acceptor when the donor-acceptor pair is in close proximity, which resulting in a fluorescent signal. Unlabeled antibody or Bicycle peptides competing with the IgG-d2 acceptor reduce the overall signal in a dose-dependent manner which enables IC50-values to be determined.

The experimental procedure followed the manufacturer's instructions detailed in the product information for the CD16 (FCγRIIIa) kit (Cisbio-6FR3APAG). Two human IgG antibodies were included as controls (Cisbio-6FR3APAG top concentration 267 nM and R&D systems-1-001-A top concentration 3000 nM). The Tag-lite buffer was prepared to generate the IgG-d2 acceptor solution. Bicycle peptides were dispensed using Echo compound handler at a top concentration of 20 μM. The competing unlabeled antibodies and bicyclic peptides were added in a three-fold dilution series in Tag-lite buffer into PerkinElmer-ProxiPlate-384 Plus.

The HEK293 cell expressing Terbium-labelled CD16a were thawed and washed in PBS and added to the wells of PerkinElmer-ProxiPlate-384 Plus containing the competitor compounds. Finally, the acceptor IgG-d2 reagent was added and the plate was incubated for 2 h at RT. The FRET signal was read on the Envision plate reader using Ex 340 nM and Em 655/620 nM settings. The data was plotted in Graph pad prism and IC50-values were determined to indicate unlabeled antibody or bicyclic peptide competition for cellular binding to the CD16a receptor.

Selected peptides of the invention were tested in the above-mentioned assay and the results are shown in Table 8:

TABLE 8
IgG Competition Assay Data for Selected
Bicyclic Peptides of the Invention (HTRF)

| Bicyclic | | |
|---|---|---|
| Peptide | IC50 (nM) | |
| BCY11808 | 9445 | |
| BCY14950 | 2035 ± 635 | (n = 5) |
| BCY14953 | 1904 | |
| BCY14955 | 1407 ± 494 | (n = 2) |
| BCY14956 | 1405 ± 204 | (n = 2) |
| BCY15044 | 7462 | |
| BCY15045 | 4022 | |
| BCY15685 | 7556 | |
| BCY16493 | 4794 | |
| BCY16599 | 1529 | |
| BCY16608 | 1802 | |
| BCY19033 | 1423 | |
| BCY19034 | 1608 | |
| BCY19035 | 2006 | |
| BCY19036 | 953 ± 12 | |
| BCY19037 | 2347 | |
| BCY19596 | 1619 ± 107 | (n = 2) |
| BCY20366 | 685 | |
| BCY20380 | 898 | |
| BCY20382 | 749 | |
| BCY20383 | 1107 | |
| BCY20384 | 526 | |
| BCY20385 | 229 ± 132 | (n = 2) |

| | |
|---|---|
| BCY20386 | 1060 |
| BCY20387 | 278 |
| BCY20389 | 851 |
| BCY20526 | 590 |
| BCY20527 | 651 |
| BCY20528 | 617 |
| BCY21689 | 1241 |
| BCY21690 | 1088 |
| BCY21691 | 153 |
| BCY21692 | 88 |
| BCY21693 | 377 |
| BCY21694 | 166 |
| BCY21695 | 100 |
| BCY21696 | 102 |
| BCY21697 | 120 |

### LIST OF REFERENCES

Muta T et al, Nature. 1994 Mar. 3; 368(6466):70-3. doi: 10.1038/368070a0. Erratum in: Nature. 1994 May 26; 369(6478):340. PMID: 8107887.
Ravetch J V, Bolland S. IgG Fc receptors. Annu Rev Immunol. 2001; 19:275-90. doi: 10.1146/annurev.immunol.19.1.275. PMID: 11244038.
Arnould L et al, Br J Cancer. 2006 Jan. 30; 94(2):259-67. doi: 10.1038/sj.bjc.6602930. PMID: 16404427; PMCID: PMC2361112.
Zhang W et al, J Clin Oncol. 2007 Aug. 20; 25(24):3712-8. doi: 10.1200/JCO.2006.08.8021. 10 PMID: 17704420.
Zhang X et al, Mol Immunol. 2020 March; 119:48-58. doi: 10.1016/j.molimm.2020.01.009. Epub 2020 Jan. 21. PMID: 31978707.
Wu S Y et al, Mol Cancer. 2020 Aug. 6; 19(1):120. doi: 10.1186/s12943-020-01238-x. PMID: 32762681; PMCID: PMC7409673.
Manches O et al, Blood. 2003 Feb. 1; 101(3):949-54. doi: 10.1182/blood-2002-02-0469. Epub 2002 Oct. 3. PMID: 12393572.
Clynes R A et al, Nat Med. 2000 April; 6(4):443-6. doi: 10.1038/74704. PMID: 10742152.
Riddle D S et al, Hum Gene Ther. 2005 July; 16(7):830-44. doi: 10.1089/hum.2005.16.830. PMID: 16000065.
Rataj F et al, Br J Cancer. 2019 January; 120(1):79-87. doi: 10.1038/s41416-018-0341-1. Epub 2018 Nov. 15. PMID: 30429531; PMCID: PMC6325122.
Caratelli S et al, Front Immunol. 2017 Apr. 27; 8:457. doi: 10.3389/fimmu.2017.00457. PMID: 28496440; PMCID: PMC5406408.
Salmon J E et al, Arthritis Rheum. 2001 April; 44(4):739-50. doi: 10.1002/1529-0131(200104)44:4<739::AID-ANR129>3.0. CO; 2-O. PMID: 11315912.
Morgan A W et al, Rheumatology (Oxford). 2003 April; 42(4):528-33. doi: 10.1093/rheumatology/keg169. PMID: 12649399.
Wu J et al, J Clin Invest. 1997 Sep. 1; 100(5):1059-70. doi: 10.1172/JC1119616. PMID: 9276722; PMCID: PMC508280.
Nabbe K C et al, Arthritis Rheum. 2003 January; 48(1):255-65. doi: 10.1002/art.10721. PMID: 12528127.
Kleinau S et al, J Exp Med. 2000 May 1; 191(9):1611-6. doi: 10.1084/jem.191.9.1611. PMID: 10790435; PMCID: PMC2213438.
Bolland S et al, Immunity. 2000 August; 13(2):277-85. doi: 10.1016/s1074-7613(00)00027-3. PMID: 10981970.
Salmon J E et al, Arthritis Rheum. 2001 April; 44(4):739-50. doi: 10.1002/1529-0131(200104)44:4<739::AID-ANR129>3.0. CO; 2-O. PMID: 11315912.
Boruchov A M et al, J Clin Invest. 2005 October; 115(10):2914-23. doi: 10.1172/JC124772. Epub 2005 Sep. 15. PMID: 16167082; PMCID: PMC1201664.
Chen J Y et al, Arthritis Rheumatol. 2014 November; 66(11):3113-21. doi: 10.1002/art.38813. PMID: 25154742; PMCID: PMC4232894.
Henriques A et al, Rheumatol Int. 2012 April; 32(4):863-9. doi: 10.1007/s00296-010-1709-6. Epub 2011 Jan. 8. PMID: 21221593.
Chen J Y et al, Arthritis Rheumatol. 2014 November; 66(11):3113-21. doi: 10.1002/art.38813. PMID: 25154742; PMCID: PMC4232894.
Clarkson S B et al, N Engl J Med. 1986 May 8; 314(19):1236-9. doi: 10.1056/NEJM198605083141907. PMID: 2939345.
Flaherty M M et al, Toxicol Sci. 2012 January; 125(1):299-309. doi: 10.1093/toxsci/kfr278. Epub 2011 Oct. 24. PMID: 22025730.
Anthony R M et al, Science. 2008 Apr. 18; 320(5874):373-6. doi: 10.1126/science.1154315. PMID: 18420934; PMCID: PMC2409116.
Debré M et al, Lancet. 1993 Oct. 16; 342(8877):945-9. doi: 10.1016/0140-6736(93)92000-j. PMID: 8105212.
Hsu C H et al, Pediatr Infect Dis J. 1993 June; 12(6):509-12. doi: 10.1097/00006454-199306000-00010. PMID: 8345983.
Ortiz D F et al, Sci Transl Med. 2016 Nov. 16; 8(365):365ra158. doi: 10.1126/scitranslmed.aaf9418. PMID: 27856797.
Li X et al, Expert Opin Ther Targets. 2014 March; 18(3):335-50. doi: 10.1517/14728222.2014.877891. PMID: 24521454; PMCID: PMC4019044.
Pasero C et al, Oncotarget. 2015 Jun. 10; 6(16):14360-73. doi: 10.18632/oncotarget.3965. PMID: 25961317; PMCID: PMC4546472.
Stringaris K et al, Haematologica. 2014 May; 99(5):836-47. doi: 10.3324/haematol.2013.087536. Epub 2014 Jan. 31. PMID: 24488563; PMCID: PMC4008119.
Chinnery F et al, Oncoimmunology. 2012 Sep. 1; 1(6):874-883. doi: 10.4161/onci.20636. PMID: 23162755; PMCID: PMC3489743.
Arnon T I et al, Blood. 2004 Jan. 15; 103(2):664-72. doi: 10.1182/blood-2003-05-1716. Epub 2003 Sep. 22. PMID: 14504081.
Moretta L et al, Semin Immunol. 2006 June; 18(3):151-8. doi: 10.1016/j.smim.2006.03.002. Epub 2006 May 26. PMID: 16730454.
Costello R T et al, Blood. 2002 May 15; 99(10):3661-7. doi: 10.1182/blood.v99.10.3661. PMID: 11986221.
Garcia-Iglesias T et al, BMC Cancer. 2009 Jun. 16; 9:186. doi: 10.1186/1471-2407-9-186. PMID: 19531227; PMCID: PMC2704222.
Nayyar G et al, Front Oncol. 2019 Feb. 11; 9:51. doi: 10.3389/fonc.2019.00051. PMID: 30805309; PMCID: PMC6378304.
Stojanovic A et al, J Innate Immun. 2011; 3(4):355-64. doi: 10.1159/000325465. Epub 2011 Apr. 18. PMID: 21502747.
Sordo-Bahamonde C et al, Cancers (Basel). 2020 Apr. 7; 12(4):893. doi: 10.3390/cancers12040893. PMID: 32272610; PMCID: PMC7226138.
Watanabe M et al, Dis Esophagus. 2010 November; 23(8):675-81. doi: 10.1111/j.1442-2050.2010.01073.x. PMID: 20545975.
Izawa S et al, Cancer Immunol Immunother. 2011 December; 60(12):1801-10. doi: 10.1007/s00262-011-1082-7. Epub 2011 Aug. 3. PMID: 21811786.
Koo K C et al, PLoS One. 2013 Nov. 4; 8(11):e78049. doi: 10.1371/journal.pone.0078049. PMID: 24223759; PMCID: PMC3817174.
Sun C et al, Cell Mol Immunol. 2015 May; 12(3):292-302. doi: 10.1038/cmi.2014.91. Epub 2014 Oct. 13. PMID: 25308752; PMCID: PMC4654321.
Hasmim M et al, Front Immunol. 2015 Sep. 23; 6:482. doi: 10.3389/fimmu.2015.00482. PMID: 26441986; PMCID: PMC4585210.
Han B et al, J Immunol Res. 2018 Sep. 3; 2018:6248590. doi: 10.1155/2018/6248590. PMID: 30255106; PMCID: PMC6140275.
Stringaris K et al, Haematologica. 2014 May; 99(5):836-47. doi: 10.3324/haematol.2013.087536. Epub 2014 Jan. 31. PMID: 24488563; PMCID: PMC4008119.
Rocca Y S et al, Front Immunol. 2016 Oct. 10; 7:413. doi: 10.3389/fimmu.2016.00413. PMID: 27777574; PMCID: PMC5056190.
Gauthier L et al, Cell. 2019 Jun. 13; 177(7):1701-1713.e16. doi: 10.1016/j.cell.2019.04.041. Epub 2019 May 30. PMID: 31155232.
Fauriat C et al, Blood. 2007 Jan. 1; 109(1):323-30. doi: 10.1182/blood-2005-08-027979. Epub 2006 Aug. 29. PMID: 16940427.
Mamessier E et al, Cancer Res. 2011 Nov. 1; 71(21):6621-32. doi: 10.1158/0008-5472.CAN-11-0792. Epub 2011 Sep. 21. PMID: 21937679.
Platonova S et al, Cancer Res. 2011 Aug. 15; 71(16):5412-22. doi: 10.1158/0008-5472.CAN-10-4179. Epub 2011 Jun. 27. PMID: 21708957.
Levi I et al, Oncotarget. 2015 May 30; 6(15):13835-43. doi: 10.18632/oncotarget.3453. PMID: 26079948; PMCID: PMC4537053.
Kim H S et al, Immunity. 2010 Feb. 26; 32(2):175-86. doi: 10.1016/j.immuni.2010.02.004. PMID: 20189481; PMCID: PMC2843589.
MacFarlane A W 4th et al, Oncoimmunology. 2017 May 19; 6(7):e1330235. doi: 10.1080/2162402X.2017.1330235. PMID: 28811973; PMCID: PMC5543845.
Pasero C et al, Oncotarget. 2015 Jun. 10; 6(16):14360-73. doi: 10.18632/oncotarget.3965. PMID: 25961317; PMCID: PMC4546472.
Stringaris K et al, Haematologica. 2014 May; 99(5):836-47. doi: 10.3324/haematol.2013.087536. Epub 2014 Jan. 31. PMID: 24488563; PMCID: PMC4008119.

1. A peptide ligand specific for natural killer (NK) cells comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

2. The peptide ligand as defined in claim 1, wherein said reactive groups comprise cysteine or penicillamine residues.

3. The peptide ligand as defined in claim 1 or claim 2, wherein said bicyclic peptide is specific for a natural cytotoxicity receptor present on the NK cell surface, such as NKp30, NKp44 and NKp46, in particular NKp46.

4. The peptide ligand as defined in claim 3, wherein said loop sequences comprise 4, 5, 6 or 7 amino acids.

5. The peptide ligand as defined in claim 3 or claim 4, wherein said loop sequences comprise three cysteine residues separated by two loop sequences the first of which consists of 5 amino acids and the second of which consists of 4 amino acids, such as:
wherein the bicyclic peptide ligand comprises an amino acid sequence which is selected from:

| (SEQ ID NO: 1) | |
|---|---|
| | CᵢNLQKPCᵢᵢMKYPCᵢᵢᵢ; |
| | and |
| (SEQ ID NO: 2) | |
| | CᵢNLQNPCᵢᵢMKFPCᵢᵢᵢ; |

wherein Cᵢ, Cᵢᵢ and Cᵢᵢᵢ represent first, second and third cysteine residues, respectively, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 1)-A-Sar₆-KFI (herein referred to as BCY14654);
A-(SEQ ID NO: 1)-A (herein referred to as BCY14456);
A-(SEQ ID NO: 2)-A-Sar₆-KFI (herein referred to as BCY15125);
A-(SEQ ID NO: 2)-A (herein referred to as BCY15102); and
A-(SEQ ID NO: 2)-A-[K(PYA)] (herein referred to as BCY18004),
wherein PYA represents pentynoic acid, Sar represents sarcosine and FI represents fluorescein.

6. The peptide ligand as defined in claim 3 or claim 4, wherein said loop sequences comprise three cysteine residues separated by two loop sequences the first of which consists of 5 amino acids and the second of which consists of 6 amino acids, such as:
wherein the bicyclic peptide ligand comprises an amino acid sequence which is selected from:

| (SEQ ID NO: 3) | |
|---|---|
| | CᵢNLQAPCᵢᵢMQTGKVCᵢᵢᵢ; |
| | (SEQ ID NO: 4; hereᵢn |
| | referred to as BCY18295 |
| | when complexed wᵢth TATA) |
| | CᵢNLQAPCᵢᵢMLTGKVCᵢᵢᵢ; |
| (SEQ ID NO: 63) | |
| | CᵢNLQQACᵢᵢLHTGKVCᵢᵢᵢ; |
| (SEQ ID NO: 64) | |
| | CᵢNLQAACᵢᵢMLTGKVCᵢᵢᵢ; |
| (SEQ ID NO: 65) | |
| | CᵢNLQAPCᵢᵢMATGKVCᵢᵢᵢ; |
| (SEQ ID NO: 66) | |
| | CᵢNLQAPCᵢᵢMLAGKVCᵢᵢᵢ; |
| (SEQ ID NO: 67) | |
| | CᵢNLQAPCᵢᵢMLTAKVCᵢᵢᵢ; |
| (SEQ ID NO: 68) | |
| | CᵢNLQAPCᵢᵢMLTGAVCᵢᵢᵢ; |
| (SEQ ID NO: 69) | |
| | CᵢNLQAPCᵢᵢMLTGKACᵢᵢᵢ; |
| (SEQ ID NO: 70) | |
| | CᵢN[tBuAla]QAPCᵢᵢMLTGKVCᵢᵢᵢ; |
| (SEQ ID NO: 71) | |
| | CᵢNLQA[Sar]CᵢᵢMLTGKVCᵢᵢᵢ; |
| (SEQ ID NO: 72) | |
| | CᵢNLQA[Cis-HyP]CᵢᵢMLTGKVCᵢᵢᵢ; |
| (SEQ ID NO: 73) | |
| | CᵢNLQA[HyP]CᵢᵢMLTGKVCᵢᵢᵢ; |
| (SEQ ID NO: 74) | |
| | CᵢNLQA[NMeAla]CᵢᵢMLTGKVCᵢᵢᵢ; |
| (SEQ ID NO: 75) | |
| | CᵢNLQAPCᵢᵢLLTGKVCᵢᵢᵢ; |
| (SEQ ID NO: 76) | |
| | CᵢNLQAPCᵢᵢ[Nle]LTGKVCᵢᵢᵢ; |
| (SEQ ID NO: 77) | |
| | CᵢNLQAPCᵢᵢ[Cba]LTGKVCᵢᵢᵢ; |
| (SEQ ID NO: 78) | |
| | CᵢNLQAPCᵢᵢMETGKVCᵢᵢᵢ; |
| (SEQ ID NO: 79) | |
| | CᵢNLQAPCᵢᵢMFTGKVCᵢᵢᵢ; |
| (SEQ ID NO: 80) | |
| | CᵢNLQAPCᵢᵢMYTGKVCᵢᵢᵢ; |
| (SEQ ID NO: 81) | |
| | CᵢNLQAPCᵢᵢM[Cba]TGKVCᵢᵢᵢ; |
| (SEQ ID NO: 82) | |
| | CᵢNLQAPCᵢᵢM[tBuAla]TGKVCᵢᵢᵢ; |
| (SEQ ID NO: 83) | |
| | CᵢNLQAPCᵢᵢM[Cha]TGKVCᵢᵢᵢ; |
| (SEQ ID NO: 84) | |
| | CᵢNLQAPCᵢᵢMLT[dA]KVCᵢᵢᵢ; |
| (SEQ ID NO: 85) | |
| | CᵢNLQAPCᵢᵢMLTGRVCᵢᵢᵢ; |
| (SEQ ID NO: 86) | |
| | CᵢNLQAPCᵢᵢMLTG[Agb]VCᵢᵢᵢ; |
| (SEQ ID NO: 87) | |
| | CᵢNLQAPCᵢᵢMLTGK[tBuGly]Cᵢᵢᵢ; |
| (SEQ ID NO: 88) | |
| | CᵢNLQAPCᵢᵢMLTGK[Chg]Cᵢᵢᵢ; |
| (SEQ ID NO: 89) | |
| | CᵢNLQAPCᵢᵢMLTGK[Cbg]Cᵢᵢᵢ; |
| (SEQ ID NO: 90) | |
| | CᵢNLQAPCᵢᵢMLTG[HArg]VCᵢᵢᵢ; |
| (SEQ ID NO: 91) | |
| | CᵢNLQA[Aze]CᵢᵢMLTGKVCᵢᵢᵢ; |
| (SEQ ID NO: 92) | |
| | CᵢNLQA[Pip]CᵢᵢMLTGKVCᵢᵢᵢ; |
| (SEQ ID NO: 93) | |
| | CᵢNLQAPCᵢᵢMVTGKVCᵢᵢᵢ; |
| (SEQ ID NO: 94) | |
| | CᵢNLQAPCᵢᵢM[Nle]TGKVCᵢᵢᵢ; |
| (SEQ ID NO: 95) | |
| | CᵢNLQAPCᵢᵢM[Nva]TGKVCᵢᵢᵢ; |
| (SEQ ID NO: 96) | |
| | CᵢNLQAPCᵢᵢMLTGK[Allolle]Cᵢᵢᵢ; |
| (SEQ ID NO: 97) | |
| | CᵢNLQAPCᵢᵢMLTGK[EPA]Cᵢᵢᵢ; |
| (SEQ ID NO: 98) | |
| | CᵢNLQQPCᵢᵢMLTGKVCᵢᵢᵢ; |
| (SEQ ID NO: 99) | |
| | CᵢNLQA[Nva]CᵢᵢMLTGKVCᵢᵢᵢ; |
| (SEQ ID NO: 100) | |
| | CᵢNLQAPCᵢᵢMHTGKVCᵢᵢᵢ; |
| (SEQ ID NO: 101) | |
| | CᵢNLQAPCᵢᵢLHTGKVCᵢᵢᵢ; |
| | and |
| (SEQ ID NO: 102) | |
| | CᵢNLQQACᵢᵢMQTGKVCᵢᵢᵢ; |

wherein Cᵢ, Cᵢᵢ and Cᵢᵢᵢ represent first, second and third cysteine residues, respectively, and wherein Cba represents cyclobutylalanine, tBuAla represents β-t-Butyl-L-alanine, Sar represents sarcosine, Cis-HyP represents L-4-cis-hydroxyproline, HyP represents hydroxyproline, NMeAla represents N-methylalanine, Ne represents norleucine, dA represents D-alanine, Agb represents norarginine, tBuGly represents tert-butylglycine, Cha represents 3-cyclohexyl-L-alanine, Chg represents cyclohexylglycine, Cbg represents cyclobutylglycine, HArg represents homoarginie, Aze represents azetidine-2-carboxylic acid, Pip represents pipecolic acid, Nva represents norvaline, Allolle represents L-allo-isoleucine, and EPA represents diethyl-L-alanine, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 3)-A-[K(PYA)] (herein referred to as BCY15687);
A-(SEQ ID NO: 3)-A (herein referred to as BCY15098);
A-(SEQ ID NO: 4)-A-Sar₆-KFI (herein referred to as BCY15115);
A-(SEQ ID NO: 4)-A (herein referred to as BCY15099);
Ac-(SEQ ID NO: 4) (herein referred to as BCY18293);
Ac-A-(SEQ ID NO: 4)-A (herein referred to as BCY18294);
(SEQ ID NO: 4)-A (herein referred to as BCY19694);
K-(SEQ ID NO: 4)-A (herein referred to as BCY19695);
dK-(SEQ ID NO: 4)-A (herein referred to as BCY19696);
Dap-(SEQ ID NO: 4)-A (herein referred to as BCY19697);
Dab-(SEQ ID NO: 4)-A (herein referred to as BCY19698);
GABA-(SEQ ID NO: 4)-A (herein referred to as BCY19699);
A-(SEQ ID NO: 63)-A (herein referred to as BCY14454);
A-(SEQ ID NO: 64)-A (herein referred to as BCY18299);
A-(SEQ ID NO: 65)-A (herein referred to as BCY18301);
A-(SEQ ID NO: 66)-A (herein referred to as BCY18302);
A-(SEQ ID NO: 67)-A (herein referred to as BCY18303);
A-(SEQ ID NO: 68)-A (herein referred to as BCY18304);
A-(SEQ ID NO: 69)-A (herein referred to as BCY18305);
A-(SEQ ID NO: 70)-A (herein referred to as BCY18307);
A-(SEQ ID NO: 71)-A (herein referred to as BCY18309);
A-(SEQ ID NO: 72)-A (herein referred to as BCY18310);
A-(SEQ ID NO: 73)-A (herein referred to as BCY18311);
A-(SEQ ID NO: 74)-A (herein referred to as BCY18312);
A-(SEQ ID NO: 75)-A (herein referred to as BCY18313);
A-(SEQ ID NO: 76)-A (herein referred to as BCY18314);
A-(SEQ ID NO: 77)-A (herein referred to as BCY18315);
A-(SEQ ID NO: 78)-A (herein referred to as BCY18316);
A-(SEQ ID NO: 79)-A (herein referred to as BCY18317);
A-(SEQ ID NO: 80)-A (herein referred to as BCY18318);
A-(SEQ ID NO: 81)-A (herein referred to as BCY18319);
A-(SEQ ID NO: 82)-A (herein referred to as BCY18320);
A-(SEQ ID NO: 83)-A (herein referred to as BCY18321);
A-(SEQ ID NO: 84)-A (herein referred to as BCY18322);
A-(SEQ ID NO: 85)-A (herein referred to as BCY18323);
A-(SEQ ID NO: 86)-A (herein referred to as BCY18324);
A-(SEQ ID NO: 87)-A (herein referred to as BCY18325);
A-(SEQ ID NO: 88)-A (herein referred to as BCY18326);
A-(SEQ ID NO: 89)-A (herein referred to as BCY18327);
A-(SEQ ID NO: 90)-A (herein referred to as BCY18328);
A-(SEQ ID NO: 91)-A (herein referred to as BCY19700);
A-(SEQ ID NO: 92)-A (herein referred to as BCY19701);
A-(SEQ ID NO: 93)-A (herein referred to as BCY19702);
A-(SEQ ID NO: 94)-A (herein referred to as BCY19703);
A-(SEQ ID NO: 95)-A (herein referred to as BCY19704);
A-(SEQ ID NO: 96)-A (herein referred to as BCY19706);
A-(SEQ ID NO: 97)-A (herein referred to as BCY19707);
A-(SEQ ID NO: 98)-A (herein referred to as BCY19708);
A-(SEQ ID NO: 99)-A (herein referred to as BCY19710);
A-(SEQ ID NO: 100)-A (herein referred to as BCY19711);
A-(SEQ ID NO: 101)-A (herein referred to as BCY19712); and
A-(SEQ ID NO: 102)-A (herein referred to as BCY19713),
wherein PYA represents pentynoic acid, Sar represents sarcosine, FI represents fluorescein dK represents D-lysine, Dap represents L-2,3-diaminopropionic acid, Dab represents L-2,4-diaminobutyric acid, and GABA represents γ-aminobutyric acid.

7. The peptide ligand as defined in claim 3 or claim 4, wherein said loop sequences comprise three cysteine residues separated by two loop sequences the first of which consists of 5 amino acids and the second of which consists of 7 amino acids, such as:
wherein the bicyclic peptide ligand comprises an amino acid sequence which is selected from:

| (SEQ ID NO: 5) | |
|---|---|
| | CᵢLLHDHCᵢᵢPNTHPKLCᵢᵢᵢ; |
| (SEQ ID NO: 6) | |
| | CᵢLLHEHCᵢᵢPNTHPKMCᵢᵢᵢ; |
| (SEQ ID NO: 7) | |
| | CᵢLLHDHCᵢᵢPNSHPEICᵢᵢᵢ; |
| (SEQ ID NO: 8) | |
| | CᵢLLHDHCᵢᵢPNTHPIICᵢᵢᵢ; |
| | and |
| (SEQ ID NO: 103) | |
| | CᵢLLHDHCᵢᵢPNTHPKMCᵢᵢᵢ; |

wherein Cᵢ, Cᵢᵢ and Cᵢᵢᵢ represent first, second and third cysteine residues, respectively, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 5)-A (herein referred to as BCY15103);
A-(SEQ ID NO: 5)-A-[K(PYA)] (herein referred to as BCY18005);
A-(SEQ ID NO: 5)-A-Sar₆-KFI (herein referred to as BCY15118);
A-(SEQ ID NO: 6)-A (herein referred to as BCY15104);
A-(SEQ ID NO: 6)-A-Sar₆-KFI (herein referred to as BCY15119);
A-(SEQ ID NO: 7)-A (herein referred to as BCY15105);
A-(SEQ ID NO: 7)-A-Sar₆-KFI (herein referred to as BCY15120);
A-(SEQ ID NO: 8)-A (herein referred to as BCY15106);
A-(SEQ ID NO: 8)-A-Sar₆-KFI (herein referred to as BCY15121); and
A-(SEQ ID NO: 103)-A (herein referred to as BCY14457),
wherein PYA represents pentynoic acid, Sar represents sarcosine and FI represents fluorescein.

8. The peptide ligand as defined in claim 3 or claim 4, wherein said loop sequences comprise three cysteine or penicillamine residues separated by two loop sequences the first of which consists of 6 amino acids and the second of which consists of 4 amino acids, such as:
wherein the bicyclic peptide ligand comprises an amino acid sequence which is selected from:

| (SEQ ID NO: 9) | |
|---|---|
| | CᵢYLPDWLCᵢᵢGDEYCᵢᵢᵢ; |
| (SEQ ID NO: 10) | |
| | CᵢYLPDYLCᵢᵢGDEYCᵢᵢᵢ; |
| | (SEQ ID NO: 11; herein referred |
| | to as BCY18262 when complexed |
| | with TATA) |
| | CᵢDLTTHNCᵢᵢQWGICᵢᵢᵢ; |
| | (SEQ ID NO: 12; herein referred |
| | to as BCY15633 when complexed |
| | with TATA) |
| | CᵢYLPDYLCᵢᵢGDEYCᵢᵢᵢ; |
| (SEQ ID NO: 13) | |
| | CᵢYLPDWLCᵢᵢGDEYCᵢᵢᵢ; |
| (SEQ ID NO: 14) | |
| | Cᵢ[2,6DiMeTyr]LPDYLCᵢᵢGDEYCᵢᵢᵢ; |
| (SEQ ID NO: 15) | |
| | CᵢY[tBuAla]PDYLCᵢᵢGDEYCᵢᵢᵢ; |
| | (SEQ ID NO: 16) |
| | CᵢY[Cba]PDYLCᵢᵢGDEYCᵢᵢᵢ; |
| (SEQ ID NO: 17) | |
| | CᵢYL[HyP]DYLCᵢᵢGDEYCᵢᵢᵢ; |
| (SEQ ID NO: 18) | |
| | CᵢYL[Aze]DYLCᵢᵢGDEYCᵢᵢᵢ; |
| (SEQ ID NO: 19) | |
| | CᵢYL[Aib]DYLCᵢᵢGDEYCᵢᵢᵢ; |
| (SEQ ID NO: 20) | |
| | CᵢYLPD[1Nal]LCᵢᵢGDEYCᵢᵢᵢ; |
| (SEQ ID NO: 21) | |
| | CᵢYLPD[2Nal]LCᵢᵢGDEYCᵢᵢᵢ; |
| (SEQ ID NO: 22) | |
| | CᵢYLPD[3Pal]LCᵢᵢGDEYCᵢᵢᵢ; |
| (SEQ ID NO: 23) | |
| | CᵢYLPD[2,6DᵢMeTyr]LCᵢᵢGDEYCᵢᵢᵢ; |
| (SEQ ID NO: 24) | |
| | CᵢYLPDW[tBuAla]CᵢᵢGDEYCᵢᵢᵢ; |
| (SEQ ID NO: 25) | |
| | CᵢYLPDW[Cba]CᵢᵢGDEYCᵢᵢᵢ; |
| (SEQ ID NO: 26) | |
| | CᵢYLPDYLCᵢᵢ[dA]DEYCᵢᵢᵢ; |
| (SEQ ID NO: 27) | |
| | CᵢYLPDYLCᵢᵢGDE[2,6DiMeTyr]Cᵢᵢᵢ; |
| (SEQ ID NO: 28) | |
| | CᵢALPDYLCᵢᵢGDEYCᵢᵢᵢ; |
| (SEQ ID NO: 29) | |
| | CᵢYAPDYLCᵢᵢGDEYCᵢᵢᵢ; |
| (SEQ ID NO: 30) | |
| | CᵢYLADYLCᵢᵢGDEYCᵢᵢᵢ; |
| (SEQ ID NO: 31) | |
| | CᵢYLPAYLCᵢᵢGDEYCᵢᵢᵢ; |
| (SEQ ID NO: 32) | |
| | CᵢYLPDALCᵢᵢGDEYCᵢᵢᵢ; |
| (SEQ ID NO: 33) | |
| | CᵢYLPDYACᵢᵢGDEYCᵢᵢᵢ; |
| (SEQ ID NO: 34) | |
| | CᵢYLPDYLCᵢᵢADEYCᵢᵢᵢ; |
| (SEQ ID NO: 35) | |
| | CᵢYLPDYLCᵢᵢGAEYCᵢᵢᵢ; |
| (SEQ ID NO: 36) | |
| | CᵢYLPDYLCᵢᵢGDAYCᵢᵢᵢ; |
| (SEQ ID NO: 37) | |
| | CᵢYLPDYLCᵢᵢGDEACᵢᵢᵢ; |
| (SEQ ID NO: 38) | |
| | CᵢY[C5A]PDYLCᵢᵢGDEYCᵢᵢᵢ; |
| (SEQ ID NO: 39) | |
| | CᵢY[Cha]PDYLCᵢᵢGDEYCᵢᵢᵢ; |
| (SEQ ID NO: 40) | |
| | CᵢY[Cba]PDYLCᵢᵢ[dA]DEYCᵢᵢᵢ; |
| (SEQ ID NO: 41) | |
| | CᵢYLPD[4FPhe]LCᵢᵢGDEYCᵢᵢᵢ; |
| (SEQ ID NO: 42) | |
| | CᵢYLPD[4MeOPhe]LCᵢᵢGDEYCᵢᵢᵢ; |
| (SEQ ID NO: 43) | |
| | CᵢYLPD[NO2Phe]LCᵢᵢGDEYCᵢᵢᵢ; |
| (SEQ ID NO: 44) | |
| | CᵢYLPD[4MePhe]LCᵢᵢGDEYCᵢᵢᵢ; |
| (SEQ ID NO: 45) | |
| | CᵢYLPD[pCoPhe]LCᵢᵢGDEYCᵢᵢᵢ; |
| (SEQ ID NO: 46) | |
| | CᵢYLPD[pCaPhe]LCᵢᵢGDEYCᵢᵢᵢ; |
| (SEQ ID NO: 47) | |
| | CᵢYLPD[4tBuPhe]LCᵢᵢGDEYCᵢᵢᵢ; |
| (SEQ ID NO: 48) | |
| | CᵢYLPD[CF3Phe]LCᵢᵢGDEYCᵢᵢᵢ; |
| (SEQ ID NO: 49) | |
| | CᵢYLPD[4CNPhe]LCᵢᵢGDEYCᵢᵢᵢ; |
| (SEQ ID NO: 50) | |
| | CᵢYLPD[4ClPhe]LCᵢᵢGDEYCᵢᵢᵢ; |
| (SEQ ID NO: 51) | |
| | CᵢYLPD[HPhe]LCᵢᵢGDEYCᵢᵢᵢ; |
| (SEQ ID NO: 52) | |
| | Cᵢ[2,6DiMeTyr]LPDWLCᵢᵢGDEYCᵢᵢᵢ; |
| (SEQ ID NO: 53) | |
| | CᵢY[Cba]PDWLCᵢᵢGDEYCᵢᵢᵢ; |
| (SEQ ID NO: 54) | |
| | CᵢYL[Aze]DWLCᵢᵢGDEYCᵢᵢᵢ; |
| (SEQ ID NO: 55) | |
| | CᵢYLPDWLCᵢᵢ[dA]DEYCᵢᵢᵢ; |
| (SEQ ID NO: 56) | |
| | CᵢSLPDYLCᵢᵢGDEYCᵢᵢᵢ; |
| (SEQ ID NO: 57) | |
| | CᵢDLTTHNCᵢᵢQWGICᵢᵢᵢ; |
| (SEQ ID NO: 58) | |
| | CᵢDLTTHNCᵢᵢLWGVCᵢᵢᵢ; |
| (SEQ ID NO: 59) | |
| | CᵢNLQLHNCᵢᵢWGVCᵢᵢᵢ; |
| (SEQ ID NO: 104) | |
| | CᵢDLTTHNCᵢᵢIWGVCᵢᵢᵢ; |
| (SEQ ID NO: 105) | |
| | CᵢNLQLHNCᵢᵢWGVCᵢᵢᵢ; |
| (SEQ ID NO: 106) | |
| | CᵢNLQLHNCᵢᵢQWGICᵢᵢᵢ; |
| (SEQ ID NO: 107) | |
| | Cᵢ[3tBuTyr]LPDYLCᵢᵢGDEYCᵢᵢᵢ; |
| (SEQ ID NO: 108) | |
| | CᵢYLPD[3tBuTyr]LCᵢᵢGDEYCᵢᵢᵢ; |
| (SEQ ID NO: 109) | |
| | CᵢYLPDYLCᵢᵢGDE[3tBuTyr]Cᵢᵢᵢ; |
| (SEQ ID NO: 110) | |
| | Cᵢ[K(PYA)]LPDYLCᵢᵢGDEYCᵢᵢᵢ; |
| (SEQ ID NO: 111) | |
| | CᵢYLP[K(PYA)]YLCᵢᵢGDEYCᵢᵢᵢ; |
| (SEQ ID NO: 112) | |
| | CᵢYLPDYLCᵢᵢ[K(PYA)]DEYCᵢᵢᵢ; |
| (SEQ ID NO: 113) | |
| | CᵢYLPDYLCᵢᵢGD[K(PYA)]YCᵢᵢᵢ; |
| (SEQ ID NO: 114) | |
| | CᵢYLPDYLCᵢᵢGDE[K(PYA)]Cᵢᵢᵢ; |
| (SEQ ID NO: 115) | |
| | CᵢYLPDYLCᵢᵢ[dK(PYA)]DEYCᵢᵢᵢ; |
| (SEQ ID NO: 116) | |
| | CᵢALTTHNCᵢᵢQWGICᵢᵢᵢ; |
| (SEQ ID NO: 117) | |
| | CᵢDLATHNCᵢᵢQWGICᵢᵢᵢ; |
| (SEQ ID NO: 118) | |
| | CᵢDLTAHNCᵢᵢQWGICᵢᵢᵢ; |
| (SEQ ID NO: 119) | |
| | CᵢDLTTHNCᵢᵢAWGICᵢᵢᵢ; |
| (SEQ ID NO: 120) | |
| | CᵢLTTHNCᵢᵢQWGACᵢᵢᵢ; |
| (SEQ ID NO: 121) | |
| | CᵢNLTTHNCᵢᵢQWGICᵢᵢᵢ; |
| (SEQ ID NO: 122) | |
| | CᵢDLQTHNCᵢᵢQWGICᵢᵢᵢ; |
| (SEQ ID NO: 123) | |
| | CᵢDLTLHNCᵢᵢQWGICᵢᵢᵢ; |
| (SEQ ID NO: 124) | |
| | CᵢDLTT[His3Me]NCᵢᵢQWGICᵢᵢᵢ; |
| (SEQ ID NO: 125) | |
| | CᵢDLTT[2Pal]NCᵢᵢQWGICᵢᵢᵢ; |
| (SEQ ID NO: 126) | |
| | CᵢDLTT[His1Me]NCᵢᵢQWGICᵢᵢᵢ; |
| (SEQ ID NO: 127) | |
| | CᵢDLTT[4ThᵢAz]NCᵢᵢQWGICᵢᵢᵢ; |
| (SEQ ID NO: 128) | |
| | CᵢDLTTHNCᵢᵢVWGICᵢᵢᵢ; |
| (SEQ ID NO: 129) | |
| | CᵢDLTTHNCᵢᵢ[Cba]WGICᵢᵢᵢ; |
| (SEQ ID NO: 130) | |
| | CᵢDLTTHNCᵢᵢQ[1Nal]GICᵢᵢᵢ; |
| (SEQ ID NO: 131) | |
| | CᵢDLTTHNCᵢᵢQ[Trp(Me)]GICᵢᵢᵢ; |
| (SEQ ID NO: 132) | |
| | CᵢDLTTHNCᵢᵢQWG[tBuGly]Cᵢᵢᵢ; |
| (SEQ ID NO: 133) | |
| | CᵢDLTTHNCᵢᵢQWG[tBuAla]Cᵢᵢᵢ; |
| (SEQ ID NO: 134) | |
| | CᵢDLTTHNCᵢᵢQWG[Chg]Cᵢᵢᵢ; |
| (SEQ ID NO: 135) | |
| | CᵢDLTTHNCᵢᵢQW[dA]ICᵢᵢᵢ; |
| (SEQ ID NO: 136) | |
| | CᵢS[Cba]PDYLCᵢᵢ[dA]DEYCᵢᵢᵢ; |
| (SEQ ID NO: 137) | |
| | Cᵢ[HSer][Cba]PDYLCᵢᵢ[dA]DEYCᵢᵢᵢ; |
| (SEQ ID NO: 138) | |
| | CᵢN[Cba]PDYLCᵢᵢ[dA]DEYCᵢᵢᵢ; |
| (SEQ ID NO: 139) | |
| | Cᵢ[4Pal][Cba]PDYLCᵢᵢ[dA]DEYCᵢᵢᵢ; |
| (SEQ ID NO: 140) | |
| | CᵢY[Cba][Cis-HyP]DYLCᵢᵢ[dA]DEYCᵢᵢᵢ; |
| (SEQ ID NO: 141) | |
| | CᵢY[Cba][NMeAla]DYLCᵢᵢ[dA]DEYCᵢᵢᵢ; |
| (SEQ ID NO: 142) | |
| | CᵢY[Cba]PNYLCᵢᵢ[dA]DEYCᵢᵢᵢ; |
| (SEQ ID NO: 143) | |
| | CᵢY[Cba]PQYLCᵢᵢ[dA]DEYCᵢᵢᵢ; |
| (SEQ ID NO: 144) | |
| | CᵢY[Cba]PDYLCᵢᵢ[dV]DEYCᵢᵢᵢ; |
| (SEQ ID NO: 145) | |
| | CᵢY[Cba]PDYLCᵢᵢ[dNva]DEYCᵢᵢᵢ; |
| (SEQ ID NO: 146) | |
| | CᵢY[Cba]PDYLCᵢᵢ[dF]DEYCᵢᵢᵢ; |
| (SEQ ID NO: 147) | |
| | CᵢY[Cba]PDYLCᵢᵢ[dCha]DEYCᵢᵢᵢ; |
| (SEQ ID NO: 148) | |
| | CᵢY[Cba]PDYLCᵢᵢ[dK(PYA)]DEYCᵢᵢᵢ; |
| (SEQ ID NO: 149) | |
| | CᵢY[Cba]PDYLCᵢᵢ[dNle]DEYCᵢᵢᵢ; |
| (SEQ ID NO: 150) | |
| | CᵢY[Cba]PD[DOPA]LCᵢᵢ[dA]DEYCᵢᵢᵢ; |
| (SEQ ID NO: 151) | |
| | CᵢY[Cba]PD[2FTyr]LCᵢᵢ[dA]DEYCᵢᵢᵢ; |
| (SEQ ID NO: 152) | |
| | CᵢY[Cba]PDY[Nle]Cᵢᵢ[dA]DEYCᵢᵢᵢ; |
| (SEQ ID NO: 153) | |
| | [Pen]Y[Cba]PDYLCᵢᵢ[dA]DEYCᵢᵢᵢ; |
| (SEQ ID NO: 154) | |
| | CᵢY[Cba]PDYL[Pen]ᵢᵢ[dA]DEYCᵢᵢᵢ; |
| (SEQ ID NO: 155) | |
| | CᵢY[Cba]PDYLCᵢᵢ[dA]DEY[Pen]ᵢᵢᵢ; |
| (SEQ ID NO: 156) | |
| | CᵢY[Cba]PDYLCᵢᵢ[dA]DEY[dCᵢᵢᵢ]; |
| (SEQ ID NO: 157) | |
| | [dCᵢ]Y[Cba]PDYLCᵢᵢ[dA]DEYCᵢᵢᵢ; |
| (SEQ ID NO: 158) | |
| | CᵢDLTTHNCᵢᵢQ[AzaTrp]GICᵢᵢᵢ; |
| (SEQ ID NO: 159) | |
| | CᵢDLTTHNCᵢᵢQ[2MeTrp]GICᵢᵢᵢ; |
| (SEQ ID NO: 160) | |
| | CᵢDLTTHNCᵢᵢQ[6MeTrp]GICᵢᵢᵢ; |
| (SEQ ID NO: 161) | |
| | CᵢDLTTHNCᵢᵢQ[7MeTrp]GICᵢᵢᵢ; |
| (SEQ ID NO: 162) | |
| | CᵢLTTHNCᵢᵢQ[6FTrp]GICᵢᵢᵢ; |
| (SEQ ID NO: 163) | |
| | CᵢDLTTHNCᵢᵢQ[5FTrp]GICᵢᵢᵢ; |
| (SEQ ID NO: 164) | |
| | CᵢDLTTHNCᵢᵢQ[Dht]GICᵢᵢᵢ; |
| (SEQ ID NO: 165) | |
| | CᵢDLTT[2FuAla]NCᵢᵢQWGICᵢᵢᵢ; |
| (SEQ ID NO: 166) | |
| | CᵢDLTTHNCᵢᵢQWG[EPA]Cᵢᵢᵢ; |
| (SEQ ID NO: 167) | |
| | CᵢDLTTHNCᵢᵢQWG[Nle]Cᵢᵢᵢ; |
| | and |
| (SEQ ID NO: 168) | |
| | CᵢDLTTHNCᵢᵢQWG[Allolle]Cᵢᵢᵢ; |

wherein Cᵢ, Penᵢ, Cᵢᵢ, Penᵢᵢ, Cᵢᵢᵢ and Penᵢᵢᵢ represent first, second and third reactive groups, respectively, wherein 2,6DiMeTyr represents 2,6-dimethyl-tyrosine, tBuAla represents t-butyl-alanine, Cba represents β-cyclobutylalanine, HyP represents hydroxyproline, Aze represents azetidine, Aib represents aminoisobutyric acid, 1NaI represents 1-naphthylalanine, 2NaI represents 2-naphthylalanine, 3Pal represents 3-pyridylalanine, C5A represents beta-cyclopentyl-L-alanine, Cha represents 3-cyclohexyl-L-alanine, 4FPhe represents 4-fluoro-L-phenylalanine, 4MeOPhe represents 4-methoxy-phenylalanine, NO2Phe represents 4-nitro-phenylalanine, 4MePhe represents 4-methyl-phenylalanine, pCoPhe represents para-carboxy-phenylalanine, pCaPhe represents p-carboxamido-phenylalanine, 4tBuPhe represents 4-tert-butyl-phenylalanine, CF3Phe represents 4-trifluoromethyl-phenylalanine, 4CNPhe represents 4-cyano-phenylalanine, 4CIPhe represents 4-chloro-phenylalanine, HPhe represents homophenylalanine, 3tBuTyr represents 3-tert-butyltyrosine, His3Me represents 3-methylhistidine, 2Pal represents 2-pyridylalanine, His1Me represents 1-methylhistidine, 4ThiAz represents beta-4-thiazol-L-ylalanine, Trp(Me) represents 1-methyltryptophan, tBuGly represents L-t-butylglycine, Chg represents cyclohexylglycine, HSer represents homoserine, NMeAla represents N-methylalanine, 4Pal represents 4-pyridylalanine, Cis-HyP represents 4-cis-hydroxyproline, dA represents D-Alanine, dV represents D-valine, dNva represents D-norvaline, dNle represents D-norleucine, dF represents D-phenylalanine, dCha represents 3-cyclohexyl-D-alanine, DOPA represents 3,4-Dihydroxyphenylalanine, 2FTyr represents 2-fluorotyrosine, Ne represents norleucine, dC represents D-cysteine, AzaTrp represents azatryptophan, 2MeTrp represents 2-methyltryptophan, 6MeTrp represents 6-methyltryptophan, 6FTrp represents 6-fluorotryptophan, 5FTrp represents 5-fluorotryptophan, Dht represents 2,3-Dihydrotryptophan, 2FuAla represents 2-furylalanine, Allolle represents L-allo-isoleucine or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 9)-A-[dK(PYA)] (herein referred to as BCY15013);
A-(SEQ ID NO: 10)-A (herein referred to as BCY18470);
A-(SEQ ID NO: 10)-A-[dK(PYA)] (herein referred to as BCY15452);
A-(SEQ ID NO: 11)-A (herein referred to as BCY15107); Ac-(SEQ ID NO: 11) (herein referred to as BCY18260);
Ac-A-(SEQ ID NO: 11)-A (herein referred to as BCY18261);
A-(SEQ ID NO: 11)-[Aib] (herein referred to as BCY19717);
(SEQ ID NO: 11)-A (herein referred to as BCY19718);
A-(SEQ ID NO: 11)-A-[dK(PYA)] (herein referred to as BCY19719);
A-(SEQ ID NO: 11)-A-[K(PYA)] (herein referred to as BCY15686);
A-(SEQ ID NO: 12)-A (herein referred to as BCY15100);
Ac-(SEQ ID NO: 12) (herein referred to as BCY15634);
Ac-A-(SEQ ID NO: 12)-A (herein referred to as BCY15635);
dA-(SEQ ID NO: 12) (herein referred to as BCY16134;
A-(SEQ ID NO: 12)-A-Sar₆-KFI (herein referred to as BCY15116);
A-(SEQ ID NO: 13)-A (herein referred to as BCY14455);
(SEQ ID NO: 13)-Sar₆-KFI (herein referred to as BCY15023);
Ac-(SEQ ID NO: 13)-Sar₆-KFI (herein referred to as BCY15024);
A-(SEQ ID NO: 13)-A-Sar₆-KFI (herein referred to as BCY14652);
A-(SEQ ID NO: 14)-A (herein referred to as BCY15636);
A-(SEQ ID NO: 15)-A (herein referred to as BCY15638);
A-(SEQ ID NO: 16)-A (herein referred to as BCY15639);
A-(SEQ ID NO: 17)-A (herein referred to as BCY15640);
A-(SEQ ID NO: 18)-A (herein referred to as BCY15641);
A-(SEQ ID NO: 19)-A (herein referred to as BCY15642);
A-(SEQ ID NO: 20)-A- (herein referred to as BCY15643);
A-(SEQ ID NO: 21)-A (herein referred to as BCY15644);
A-(SEQ ID NO: 22)-A (herein referred to as BCY15645);
A-(SEQ ID NO: 23)-A (herein referred to as BCY15646);
A-(SEQ ID NO: 24)-A (herein referred to as BCY15648);
A-(SEQ ID NO: 25)-A (herein referred to as BCY15649);
A-(SEQ ID NO: 26)-A (herein referred to as BCY15650);
A-(SEQ ID NO: 27)-A (herein referred to as BCY15651);
A-(SEQ ID NO: 28)-A (herein referred to as BCY15653);
A-(SEQ ID NO: 29)-A (herein referred to as BCY15654);
A-(SEQ ID NO: 30)-A (herein referred to as BCY15655);
A-(SEQ ID NO: 31)-A (herein referred to as BCY15656);
A-(SEQ ID NO: 32)-A (herein referred to as BCY15657);
A-(SEQ ID NO: 33)-A (herein referred to as BCY15658);
A-(SEQ ID NO: 34)-A (herein referred to as BCY15659);
A-(SEQ ID NO: 35)-A (herein referred to as BCY15660);
A-(SEQ ID NO: 36)-A (herein referred to as BCY15661);
A-(SEQ ID NO: 37)-A (herein referred to as BCY15662);
A-(SEQ ID NO: 38)-A (herein referred to as BCY16130);
A-(SEQ ID NO: 39)-A (herein referred to as BCY16131);
A-(SEQ ID NO: 40)-A (herein referred to as BCY16132);
Ac-(SEQ ID NO: 40) (herein referred to as BCY16133);
Ac-(SEQ ID NO: 40)-[K(PYA)] (herein referred to as BCY17224);
A-(SEQ ID NO: 41)-A (herein referred to as BCY16135);
A-(SEQ ID NO: 42)-A (herein referred to as BCY16136);
A-(SEQ ID NO: 43)-A (herein referred to as BCY16137);
A-(SEQ ID NO: 44)-A (herein referred to as BCY16138);
A-(SEQ ID NO: 45)-A (herein referred to as BCY16139);
A-(SEQ ID NO: 46)-A (herein referred to as BCY16140);
A-(SEQ ID NO: 47)-A (herein referred to as BCY16141);
A-(SEQ ID NO: 48)-A (herein referred to as BCY16142);
A-(SEQ ID NO: 49)-A (herein referred to as BCY16143);
A-(SEQ ID NO: 50)-A (herein referred to as BCY16144);
A-(SEQ ID NO: 51)-A (herein referred to as BCY16145);
A-(SEQ ID NO: 52)-A-Sar₆-KFI (herein referred to as BCY15025);
A-(SEQ ID NO: 53)-A-Sar₆-KFI (herein referred to as BCY15028);
A-(SEQ ID NO: 54)-A-Sar₆-KFI (herein referred to as BCY15030);
A-(SEQ ID NO: 55)-A-Sar₆-KFI (herein referred to as BCY15035);
A-(SEQ ID NO: 56)-A-Sar₆-KFI (herein referred to as BCY15117);
A-(SEQ ID NO: 57)-A-Sar₆-KFI (herein referred to as BCY15122);
A-(SEQ ID NO: 58)-A (herein referred to as BCY15108);
A-(SEQ ID NO: 58)-A-Sar₆-KFI (herein referred to as BCY15123);
A-(SEQ ID NO: 59)-A-Sar₆-KFI (herein referred to as BCY15124);
A-(SEQ ID NO: 104)-A (herein referred to as BCY14458);
A-(SEQ ID NO: 105)-A (herein referred to as BCY15109);
A-(SEQ ID NO: 106)-A (herein referred to as BCY15568);
A-(SEQ ID NO: 107)-A (herein referred to as BCY15637);
A-(SEQ ID NO: 108)-A (herein referred to as BCY15647);
A-(SEQ ID NO: 109)-A (herein referred to as BCY15652);
A-(SEQ ID NO: 110)-A (herein referred to as BCY16834);
A-(SEQ ID NO: 111)-A (herein referred to as BCY16837);
A-(SEQ ID NO: 112)-A (herein referred to as BCY16840);
A-(SEQ ID NO: 113)-A (herein referred to as BCY16842);
A-(SEQ ID NO: 114)-A (herein referred to as BCY16843);
A-(SEQ ID NO: 115)-A (herein referred to as BCY17662);
A-(SEQ ID NO: 116)-A (herein referred to as BCY18263);
A-(SEQ ID NO: 117)-A (herein referred to as BCY18265);
A-(SEQ ID NO: 118)-A (herein referred to as BCY18266);
A-(SEQ ID NO: 119)-A (herein referred to as BCY18269);
A-(SEQ ID NO: 120)-A (herein referred to as BCY18272);
A-(SEQ ID NO: 121)-A (herein referred to as BCY18273);
A-(SEQ ID NO: 122)-A (herein referred to as BCY18277);
A-(SEQ ID NO: 123)-A (herein referred to as BCY18278);
A-(SEQ ID NO: 124)-A (herein referred to as BCY18279);
A-(SEQ ID NO: 125)-A (herein referred to as BCY18280);
A-(SEQ ID NO: 126)-A (herein referred to as BCY18281);
A-(SEQ ID NO: 127)-A (herein referred to as BCY18282);
A-(SEQ ID NO: 128)-A (herein referred to as BCY18285);
A-(SEQ ID NO: 129)-A (herein referred to as BCY18286);
A-(SEQ ID NO: 130)-A (herein referred to as BCY18287);
A-(SEQ ID NO: 131)-A (herein referred to as BCY18288);
A-(SEQ ID NO: 132)-A (herein referred to as BCY18289);
A-(SEQ ID NO: 133)-A (herein referred to as BCY18290);
A-(SEQ ID NO: 134)-A (herein referred to as BCY18291);
A-(SEQ ID NO: 135)-A (herein referred to as BCY18292);
Ac-(SEQ ID NO: 136) (herein referred to as BCY18433);
Ac-(SEQ ID NO: 137) (herein referred to as BCY18434);
Ac-(SEQ ID NO: 138) (herein referred to as BCY18435);
Ac-(SEQ ID NO: 139) (herein referred to as BCY18437);
Ac-(SEQ ID NO: 140) (herein referred to as BCY18439);
Ac-(SEQ ID NO: 141) (herein referred to as BCY18440);
Ac-(SEQ ID NO: 142) (herein referred to as BCY18441);
Ac-(SEQ ID NO: 143) (herein referred to as BCY18442);
Ac-(SEQ ID NO: 144) (herein referred to as BCY18443);
Ac-(SEQ ID NO: 145) (herein referred to as BCY18444);
Ac-(SEQ ID NO: 146) (herein referred to as BCY19682);
Ac-(SEQ ID NO: 147) (herein referred to as BCY19683);
Ac-(SEQ ID NO: 148) (herein referred to as BCY19684);
Ac-(SEQ ID NO: 149) (herein referred to as BCY19685);
Ac-(SEQ ID NO: 150) (herein referred to as BCY19686);
Ac-(SEQ ID NO: 151) (herein referred to as BCY19687);
Ac-(SEQ ID NO: 152) (herein referred to as BCY19688);
Ac-(SEQ ID NO: 153) (herein referred to as BCY19689);
Ac-(SEQ ID NO: 154) (herein referred to as BCY19690);
Ac-(SEQ ID NO: 155) (herein referred to as BCY19691);
Ac-(SEQ ID NO: 156) (herein referred to as BCY19692);
Ac-(SEQ ID NO: 157) (herein referred to as BCY19693);
A-(SEQ ID NO: 158)-A (herein referred to as BCY19720);
A-(SEQ ID NO: 159)-A (herein referred to as BCY19721);
A-(SEQ ID NO: 160)-A (herein referred to as BCY19722);
A-(SEQ ID NO: 161)-A (herein referred to as BCY19723);
A-(SEQ ID NO: 162)-A (herein referred to as BCY19724);
A-(SEQ ID NO: 163)-A (herein referred to as BCY19725);
A-(SEQ ID NO: 164)-A (herein referred to as BCY19730);
A-(SEQ ID NO: 165)-A (herein referred to as BCY19732);
A-(SEQ ID NO: 166)-A (herein referred to as BCY19733);
A-(SEQ ID NO: 167)-A (herein referred to as BCY19734); and
A-(SEQ ID NO: 168)-A (herein referred to as BCY19735),
wherein Aib represents aminoisobutyric acid, PYA represents pentynoic acid, Sar represents sarcosine and FI represents fluorescein.

9. The peptide ligand as defined in claim 1 or claim 2, wherein said bicyclic peptide is specific for an Fc receptor present on the NK cell surface, such as a low-affinity Fc gamma receptor (FcγR) selected from FcγRIIA, FcγRIIB, FcγRIIC, FcγRIIIA, and FcγRIIIB, in particular FcγRIIIA (also known as CD16a).

10. The peptide ligand as defined in claim 9, wherein said loop sequences comprise 3, 4, 5, 6, 7 or 8 amino acids.

11. The peptide ligand as defined in claim 9 or claim 10, wherein said loop sequences comprise three cysteine residues separated by two loop sequences the first of which consists of 4 amino acids and the second of which consists of 8 amino acids, such as:
wherein the bicyclic peptide ligand comprises an amino acid sequence which is:

| (SEQ ID NO: 265) | |
|---|---|
| | CᵢTWPYCᵢᵢKSWGDVQDCᵢᵢᵢ; |

wherein Cᵢ, Cᵢᵢ and Cᵢᵢᵢ represent first, second and third cysteine residues, respectively, or a pharmaceutically acceptable salt thereof, in particular:
wherein the molecular scaffold is TATB and the bicyclic peptide ligand additionally comprises N- and/or C-terminal additions and comprises an amino acid sequence which is selected from:
A-(SEQ ID NO: 265)-A (herein referred to as BCY15044); and
A-(SEQ ID NO: 265)-AGAAAE (herein referred to as BCY19416).

12. The peptide ligand as defined in claim 9 or claim 10, wherein said loop sequences comprise three cysteine residues separated by two loop sequences the first of which consists of 5 amino acids and the second of which consists of 4 amino acids, such as:
wherein the bicyclic peptide ligand comprises an amino acid sequence which is selected from:

| (SEQ ID NO: 60) | |
|---|---|
| | CᵢPPTVYCᵢᵢHPLDCᵢᵢᵢ; |
| (SEQ ID NO: 169) | |
| | CᵢPPAVYCᵢᵢHPLDCᵢᵢᵢ; |
| (SEQ ID NO: 170) | |
| | CᵢPPEVFCᵢᵢHPLDCᵢᵢᵢ; |
| (SEQ ID NO: 171) | |
| | CᵢPPEVYCᵢᵢHPLDCᵢᵢᵢ; |
| (SEQ ID NO: 172) | |
| | CᵢPPEVWCᵢᵢHPLDCᵢᵢᵢ; |
| (SEQ ID NO: 173) | |
| | CᵢPPQVYCᵢᵢHPLDCᵢᵢᵢ; |
| | and |
| (SEQ ID NO: 174) | |
| | CᵢPPEVWCᵢᵢHPLDCᵢᵢᵢ; |

wherein Cᵢ, Cᵢᵢ and Cᵢᵢᵢ represent first, second and third cysteine residues, respectively, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 60)-A (herein referred to as BCY11808);
A-(SEQ ID NO: 60)-A-[K(PYA)] (herein referred to as BCY15685);
A-(SEQ ID NO: 60)-A-[Sar6]-[KBiot] (herein referred to as BCY12621);
A-(SEQ ID NO: 169)-A (herein referred to as BCY11673);
A-(SEQ ID NO: 170)-A (herein referred to as BCY11809);
A-(SEQ ID NO: 171)-A (herein referred to as BCY11810);
A-(SEQ ID NO: 172)-A (herein referred to as BCY11811)
A-(SEQ ID NO: 173)-A (herein referred to as BCY11812); and
A-(SEQ ID NO: 174)-A-[Sar6]-[KBiot] (herein referred to as BCY12622);
wherein Sar represents sarcosine and Biot represents biotin.

13. The peptide ligand as defined in claim 9 or claim 10, wherein said loop sequences comprise three cysteine residues separated by two loop sequences the first of which consists of 6 amino acids and the second of which consists of 3 amino acids, such as:
wherein the bicyclic peptide ligand comprises an amino acid sequence which is:

| (SEQ ID NO: 266) | |
|---|---|
| | CᵢDWWDPGCᵢᵢTYFCᵢᵢᵢ; |

wherein Cᵢ, Cᵢᵢ and Cᵢᵢᵢ represent first, second and third cysteine residues, respectively, or a pharmaceutically acceptable salt thereof, in particular:
wherein the molecular scaffold is TATB and the bicyclic peptide ligand additionally comprises N- and/or C-terminal additions and comprises an amino acid sequence which is selected from:
A-(SEQ ID NO: 266)-A (herein referred to as BCY15045); and
A-(SEQ ID NO: 266)-AGAAAE (herein referred to as BCY19417).

14. The peptide ligand as defined in claim 9 or claim 10, wherein said loop sequences comprise three cysteine residues separated by two loop sequences the first of which consists of 7 amino acids and the second of which consists of 3 amino acids, such as:
wherein the bicyclic peptide ligand comprises an amino acid sequence which is selected from:

| (SEQ ID NO: 61) | |
|---|---|
| | CᵢRWSVEDPCᵢᵢGAWCᵢᵢᵢ; |
| (SEQ ID NO: 175) | |
| | CᵢRWS[tBuGly]EDPCᵢᵢGAWCᵢᵢᵢ; |
| (SEQ ID NO: 176) | |
| | CᵢRWSVED[HyP]CᵢᵢGAWCᵢᵢᵢ; |
| (SEQ ID NO: 177) | |
| | CᵢRWSVED[Aze]CᵢᵢGAWCᵢᵢᵢ; |
| (SEQ ID NO: 178) | |
| | CᵢRWSVEDPCᵢᵢ[dA]AWCᵢᵢᵢ; |
| (SEQ ID NO: 179) | |
| | CᵢRWSVE[Gla]PCᵢᵢGAWCᵢᵢᵢ; |
| (SEQ ID NO: 180) | |
| | CᵢRWSVED[Aze]CᵢᵢGAWCᵢᵢᵢ; |
| (SEQ ID NO: 181) | |
| | CᵢMWIRSDPCᵢᵢGAWCᵢᵢᵢ; |
| (SEQ ID NO: 182) | |
| | CᵢRWYPDDPCᵢᵢGAWCᵢᵢᵢ; |
| (SEQ ID NO: 183) | |
| | CᵢHEWHISEPCᵢᵢGAWCᵢᵢᵢ; |
| (SEQ ID NO: 184) | |
| | CᵢNWHVYEPCᵢᵢGAWCᵢᵢᵢ; |
| (SEQ ID NO: 185) | |
| | CᵢRWHFSEPCᵢᵢGAWCᵢᵢᵢ; |
| (SEQ ID NO: 186) | |
| | CᵢRWNLDDPCᵢᵢGAWCᵢᵢᵢ; |
| (SEQ ID NO: 187) | |
| | CᵢMWIRSDPCᵢᵢGAWCᵢᵢᵢ; |
| (SEQ ID NO: 188) | |
| | CᵢRWYPDDPCᵢᵢGAWCᵢᵢᵢ; |
| (SEQ ID NO: 189) | |
| | CᵢHEWHISEPCᵢᵢGAWCᵢᵢᵢ; |
| (SEQ ID NO: 190) | |
| | CᵢNWHVYEPCᵢᵢGAWCᵢᵢᵢ; |
| (SEQ ID NO: 191) | |
| | CᵢRWHFSEPCᵢᵢGAWCᵢᵢᵢ; |
| (SEQ ID NO: 192) | |
| | CᵢRWNLDDPCᵢᵢGAWCᵢᵢᵢ; |
| (SEQ ID NO: 193) | |
| | CᵢR[2MeTrp]SVEDPCᵢᵢGAWCᵢᵢᵢ; |
| (SEQ ID NO: 194) | |
| | CᵢR[6MeTrp]SVEDPCᵢᵢGAWCᵢᵢᵢ; |
| (SEQ ID NO: 195) | |
| | CᵢR[6FTrp]SVEDPCᵢᵢGAWCᵢᵢᵢ; |
| (SEQ ID NO: 196) | |
| | CᵢR[5FTrp]SVEDPCᵢᵢGAWCᵢᵢᵢ; |
| (SEQ ID NO: 197) | |
| | CᵢR[6CiTrp]SVEDPCᵢᵢGAWCᵢᵢᵢ; |
| (SEQ ID NO: 198) | |
| | CᵢR[Trp(S)]SVEDPCᵢᵢGAWCᵢᵢᵢ; |
| (SEQ ID NO: 199) | |
| | CᵢR[AzaTrp]SVEDPCᵢᵢGAWCᵢᵢᵢ; |
| (SEQ ID NO: 200) | |
| | CᵢRWSVEDPCᵢᵢGA[2MeTrp]Cᵢᵢᵢ; |
| (SEQ ID NO: 201) | |
| | CᵢRWSVEDPCᵢᵢGA[6FTrp]Cᵢᵢᵢ; |
| (SEQ ID NO: 202) | |
| | CᵢRWSVEDPCᵢᵢGA[5FTrp]Cᵢᵢᵢ; |
| (SEQ ID NO: 203) | |
| | CᵢRWSVEDPCᵢᵢGA[4MeoTrp]Cᵢᵢᵢ; |
| (SEQ ID NO: 204) | |
| | CᵢRWSVEDPCᵢᵢGA[5MeoTrp]Cᵢᵢᵢ; |
| (SEQ ID NO: 205) | |
| | CᵢRWSVEDPCᵢᵢGA[Trp(S)]Cᵢᵢᵢ; |
| (SEQ ID NO: 206) | |
| | CᵢRWSVEDPCᵢᵢGA[DOPA]Cᵢᵢᵢ; |
| (SEQ ID NO: 207) | |
| | CᵢRWYPDDPCᵢᵢGAWCᵢᵢᵢ; |
| (SEQ ID NO: 208) | |
| | CᵢRWHFSEPCᵢᵢGAWCᵢᵢᵢ; |
| (SEQ ID NO: 209) | |
| | CᵢR[2MeTrp]HFSEPCᵢᵢGAWCᵢᵢᵢ; |
| (SEQ ID NO: 210) | |
| | CᵢRWYFSEPCᵢᵢGAWCᵢᵢᵢ; |
| (SEQ ID NO: 211) | |
| | CᵢRWSFSEPCᵢᵢGAWCᵢᵢᵢ; |
| (SEQ ID NO: 212) | |
| | CᵢRWHPSEPCᵢᵢGAWCᵢᵢᵢ; |
| (SEQ ID NO: 213) | |
| | CᵢRWYPSEPCᵢᵢGAWCᵢᵢᵢ; |
| (SEQ ID NO: 214) | |
| | CᵢRWHPDEPCᵢᵢGAWCᵢᵢᵢ; |
| (SEQ ID NO: 215) | |
| | CᵢRWHPEEPCᵢᵢGAWCᵢᵢᵢ; |
| (SEQ ID NO: 216) | |
| | CᵢRWHFDEPCᵢᵢGAWCᵢᵢᵢ; |
| (SEQ ID NO: 217) | |
| | CᵢRWHFDDPCᵢᵢGAWCᵢᵢᵢ; |
| (SEQ ID NO: 218) | |
| | CᵢRWYFDEPCᵢᵢGAWCᵢᵢᵢ; |
| (SEQ ID NO: 219) | |
| | CᵢRW[His1Me]FSEPCᵢᵢGAWCᵢᵢᵢ; |
| (SEQ ID NO: 220) | |
| | CᵢRW[His3Me]FSEPCᵢᵢGAWCᵢᵢᵢ; |
| (SEQ ID NO: 221) | |
| | CᵢRW[4MeoPhe]FSEPCᵢᵢGAWCᵢᵢᵢ; |
| (SEQ ID NO: 222) | |
| | CᵢRW[DOPA]FSEPCᵢᵢGAWCᵢᵢᵢ; |
| (SEQ ID NO: 223) | |
| | CᵢRW[26DiMeTyr]FSEPCᵢᵢGAWCᵢᵢᵢ; |
| (SEQ ID NO: 224) | |
| | CᵢRW[3tBuTyr]FSEPCᵢᵢGAWCᵢᵢᵢ; |
| (SEQ ID NO: 225) | |
| | CᵢRW[4FPhe]FSEPCᵢᵢGAWCᵢᵢᵢ; |
| (SEQ ID NO: 226) | |
| | CᵢRWH[4MePhe]SEPCᵢᵢGAWCᵢᵢᵢ; |
| (SEQ ID NO: 227) | |
| | CᵢRWH[Aze]SEPCᵢᵢGAWCᵢᵢᵢ; |
| (SEQ ID NO: 228) | |
| | CᵢRWH[HyP]SEPCᵢᵢGAWCᵢᵢᵢ; |
| (SEQ ID NO: 229) | |
| | CᵢRWH[4tBuPhe]SEPCᵢᵢGAWCᵢᵢᵢ; |
| | (SEQ ID NO: 230; herein |
| | referred to as BCY21693 |
| | when complexed with TBMT) |
| | CᵢRWH[HPhe]SEPCᵢᵢGAWCᵢᵢᵢ; |
| (SEQ ID NO: 231) | |
| | CᵢRWH[4PhePro]SEPCᵢᵢGAWCᵢᵢᵢ; |
| (SEQ ID NO: 232) | |
| | CᵢRWHF[HSer]EPCᵢᵢGAWCᵢᵢᵢ; |
| (SEQ ID NO: 233) | |
| | CᵢRWHFS[Gla]PCᵢᵢGAWCᵢᵢᵢ; |
| (SEQ ID NO: 234) | |
| | CᵢRWHFS[HGlu]PCᵢᵢGAWCᵢᵢᵢ; |
| (SEQ ID NO: 235) | |
| | CᵢRWHFSEPCᵢᵢ[dA]AWCᵢᵢᵢ; |
| (SEQ ID NO: 236) | |
| | CᵢRWYFSE[Aze]Cᵢᵢ[dA]AWCᵢᵢᵢ; |
| (SEQ ID NO: 237) | |
| | CᵢRWHFDE[Aze]Cᵢᵢ[dA]AWCᵢᵢᵢ; |
| (SEQ ID NO: 238) | |
| | CᵢRWYPSE[Aze]Cᵢᵢ[dA]AWCᵢᵢᵢ; |
| (SEQ ID NO: 239) | |
| | Cᵢ[HArg]WHFSEPCᵢᵢGAWCᵢᵢᵢ; |
| (SEQ ID NO: 240) | |
| | CᵢRWH[Cis-HyP]SEPCᵢᵢGAWCᵢᵢᵢ; |
| (SEQ ID NO: 241) | |
| | CᵢRWHFSEPCᵢᵢGAWCᵢᵢᵢ; |
| (SEQ ID NO: 242) | |
| | CᵢRWHFSEPCᵢᵢGAWCᵢᵢᵢ; |
| (SEQ ID NO: 243) | |
| | CᵢRWHFSEPCᵢᵢGAWCᵢᵢᵢ; |
| (SEQ ID NO: 267) | |
| | CᵢRWH[4BnPro]SEPCᵢᵢGAWCᵢᵢᵢ; |
| | and |
| (SEQ ID NO: 268) | |
| | CᵢRWH[HPhe]SE[Aze]CᵢᵢGAWCᵢᵢᵢ; |

wherein Cᵢ, Cᵢᵢ and Cᵢᵢᵢ represent first, second and third cysteine residues, respectively, wherein Gla represents γ-carboxyglutamic acid, Aze represents azetidine-2-carboxylic acid, 2MeTrp represents 2-methyltryptophan, 6MeTrp represents 6-methyltryptophan, 6FTrp represents 6-fluorotryptophan, 5FTrp represents 5-fluorotryptophan, 6CITrp represents 6-Chloro-L-tryptophan, 5MeoTrp represents 5-Methoxy-L-tryptophan, Trp(S) represents 3-(3-Benzothienyl)-L-alanine, AzaTrp represents azatryptophan, 4MeoTrp represents 4-Methoxy-L-tryptophan, DOPA represents 3,4-Dihydroxyphenylalanine, His1Me represents 1-methylhistidine, His3Me represents 3-methylhistidine, 4MeoPhe represents 4-Methoxyphenylalanine, 26DiMeTyr represents 2,6-Dimethyl-L-tyrosine, 3tBuTyr represents 3-tert-butyltyrosine, 4FPhe represents 4-fluoro-L-phenylalanine, 4MePhe represents 4-methyl-phenylalanine, HyP represents hydroxyproline, 4tBuPhe represents 4-t-butyl-phenylalanine, HPhe represents homophenylalanine, 4PhePro represents (2S,4S)-4-PhenylProline, HSer represents homoserine, HGlu represents L-2-aminoadipic acid, dA represents D-Alanine, HArg represents homoarginine, tBuGly represents tert-butylglycine, Cis-HyP represents 4-cis-hydroxyproline, and 4BnPro represents (2S,4R)-4-benzyl-pyrrolidine-2-carboxylic acid or a pharmaceutically acceptable salt thereof, in particular:
wherein the molecular scaffold is TBMT and the bicyclic peptide ligand additionally comprises N- and/or C-terminal additions and comprises an amino acid sequence which is selected from:
A-(SEQ ID NO: 61)-A (herein referred to as BCY14950);
Ac-A-(SEQ ID NO: 61)-A (herein referred to as BCY16599);
A-(SEQ ID NO: 61)-AGAAAE (herein referred to as BCY19040);
A-(SEQ ID NO: 61)-A-[K(PYA)] (herein referred to as BCY13883);
Ac-A-(SEQ ID NO: 61)-A-[K(PYA)] (herein referred to as BCY19596);
A-(SEQ ID NO: 175)-A (herein referred to as BCY14953);
A-(SEQ ID NO: 176)-A (herein referred to as BCY14954);
[PYA]-A-(SEQ ID NO: 177) (herein referred to as BCY14955);
A-(SEQ ID NO: 178)-A (herein referred to as BCY14956);
A-(SEQ ID NO: 179)-A (herein referred to as BCY16608);
A-(SEQ ID NO: 180)-A (herein referred to as BCY16610);
A-(SEQ ID NO: 181)-A (herein referred to as BCY19032);
A-(SEQ ID NO: 182)-A (herein referred to as BCY19033);
A-(SEQ ID NO: 183)-A (herein referred to as BCY19034);
A-(SEQ ID NO: 184)-A (herein referred to as BCY19035);
A-(SEQ ID NO: 185)-A (herein referred to as BCY19036);
A-(SEQ ID NO: 186)-A (herein referred to as BCY19037);
A-(SEQ ID NO: 187)-AGAAAE (herein referred to as BCY19038);
A-(SEQ ID NO: 188)-AGAAAE (herein referred to as BCY19039);
A-(SEQ ID NO: 189)-AGAAAE (herein referred to as BCY19041);
A-(SEQ ID NO: 190)-AGAAAE (herein referred to as BCY19042);
A-(SEQ ID NO: 191)-AGAAAE (herein referred to as BCY19043);
A-(SEQ ID NO: 192)-AGAAAE (herein referred to as BCY19044);
A-(SEQ ID NO: 193)-A (herein referred to as BCY19045);
A-(SEQ ID NO: 194)-A (herein referred to as BCY19046);
A-(SEQ ID NO: 195)-A (herein referred to as BCY19048);
A-(SEQ ID NO: 196)-A (herein referred to as BCY19049);
A-(SEQ ID NO: 197)-A (herein referred to as BCY19050);
A-(SEQ ID NO: 198)-A (herein referred to as BCY19053);
A-(SEQ ID NO: 199)-A (herein referred to as BCY19055);
A-(SEQ ID NO: 200)-A (herein referred to as BCY19057);
A-(SEQ ID NO: 201)-A (herein referred to as BCY19060);
A-(SEQ ID NO: 202)-A (herein referred to as BCY19061);
A-(SEQ ID NO: 203)-A (herein referred to as BCY19063);
A-(SEQ ID NO: 204)-A (herein referred to as BCY19064);
A-(SEQ ID NO: 205)-A (herein referred to as BCY19065);
A-(SEQ ID NO: 206)-A (herein referred to as BCY19068);
A-(SEQ ID NO: 207)-A-[K(PYA)] (herein referred to as BCY20360);
A-(SEQ ID NO: 208)-A-[K(PYA)] (herein referred to as BCY20361);
A-(SEQ ID NO: 209)-A (herein referred to as BCY20363);
A-(SEQ ID NO: 210)-A (herein referred to as BCY20364);
A-(SEQ ID NO: 211)-A (herein referred to as BCY20365);
A-(SEQ ID NO: 212)-A (herein referred to as BCY20366);
A-(SEQ ID NO: 213)-A (herein referred to as BCY20367);
A-(SEQ ID NO: 214)-A (herein referred to as BCY20368);
A-(SEQ ID NO: 215)-A (herein referred to as BCY20369);
A-(SEQ ID NO: 216)-A (herein referred to as BCY20370);
A-(SEQ ID NO: 217)-A (herein referred to as BCY20371);
A-(SEQ ID NO: 218)-A (herein referred to as BCY20372);
A-(SEQ ID NO: 219)-A (herein referred to as BCY20373);
A-(SEQ ID NO: 220)-A (herein referred to as BCY20374);
A-(SEQ ID NO: 221)-A (herein referred to as BCY20375);
A-(SEQ ID NO: 222)-A (herein referred to as BCY20376);
A-(SEQ ID NO: 223)-A (herein referred to as BCY20377);
A-(SEQ ID NO: 224)-A (herein referred to as BCY20378);
A-(SEQ ID NO: 225)-A (herein referred to as BCY20379);
A-(SEQ ID NO: 226)-A (herein referred to as BCY20380);
A-(SEQ ID NO: 227)-A (herein referred to as BCY20382);
A-(SEQ ID NO: 228)-A (herein referred to as BCY20383);
A-(SEQ ID NO: 229)-A (herein referred to as BCY20384);
A-(SEQ ID NO: 230)-A (herein referred to as BCY20385);
(SEQ ID NO: 230)-A (herein referred to as BCY21690);
Ac-(SEQ ID NO: 230)-A (herein referred to as BCY21691);
A-(SEQ ID NO: 230) (herein referred to as BCY21692);
Ac-(SEQ ID NO: 230) (herein referred to as BCY21694);
[Nle]-(SEQ ID NO: 230)-A (herein referred to as BCY21695);
Ac-[Nle]-(SEQ ID NO: 230)-A (herein referred to as BCY21696);
Ac-A-(SEQ ID NO: 230)-A (herein referred to as BCY21697);
A-(SEQ ID NO: 231)-A (herein referred to as BCY20386);
A-(SEQ ID NO: 232)-A (herein referred to as BCY20388);
A-(SEQ ID NO: 233)-A (herein referred to as BCY20389);
A-(SEQ ID NO: 234)-A (herein referred to as BCY20390);
A-(SEQ ID NO: 235)-A (herein referred to as BCY20392);
A-(SEQ ID NO: 236)-A (herein referred to as BCY20394);
A-(SEQ ID NO: 237)-A (herein referred to as BCY20395);
A-(SEQ ID NO: 238)-A (herein referred to as BCY20396);
A-(SEQ ID NO: 239)-A (herein referred to as BCY20397);
A-(SEQ ID NO: 240)-A (herein referred to as BCY20526);
Ac-A-(SEQ ID NO: 241)-A (herein referred to as BCY20527);
[Aib]-(SEQ ID NO: 242)-A (herein referred to as BCY20528);
A-(SEQ ID NO: 243)-[Aib] (herein referred to as BCY20529);
A-(SEQ ID NO: 267)-A (herein referred to as BCY20387); and
A-(SEQ ID NO: 268)-A (herein referred to as BCY21689),
wherein PYA represents pentynoic acid and Aib represents aminoisobutryic acid.

15. The peptide ligand as defined in claim 9 or claim 10, wherein said loop sequences comprise three cysteine residues separated by two loop sequences the first of which consists of 8 amino acids and the second of which consists of 3 amino acids, such as:
wherein the bicyclic peptide ligand comprises an amino acid sequence which is selected from:

| | (SEQ ID NO: 62; herein |
|---|---|
| | referred to as BCY16494 |
| | when complexed with TBMT) |
| | CᵢVGLEELGPCᵢᵢSDLCᵢᵢᵢ; |
| (SEQ ID NO: 244) | |
| | CᵢVGLLELGPCᵢᵢSDLCᵢᵢᵢ; |
| (SEQ ID NO: 245) | |
| | CᵢPGLEELGPCᵢᵢSDLCᵢᵢᵢ; |
| (SEQ ID NO: 246) | |
| | CᵢV[dA]LEELGPCᵢᵢSDLCᵢᵢᵢ; |
| (SEQ ID NO: 247) | |
| | CᵢVG[tBuAla]EELGPCᵢᵢSDLCᵢᵢᵢ; |
| (SEQ ID NO: 248) | |
| | CᵢVG[Cba]EELGPCᵢᵢSDLCᵢᵢᵢ; |
| (SEQ ID NO: 249) | |
| | CᵢVGFEELGPCᵢᵢSDLCᵢᵢᵢ; |
| (SEQ ID NO: 250) | |
| | CᵢVG[1Nal]EELGPCᵢᵢSDLCᵢᵢᵢ; |
| (SEQ ID NO: 251) | |
| | CᵢVG[2Nal]EELGPCᵢᵢSDLCᵢᵢᵢ; |
| (SEQ ID NO: 252) | |
| | CᵢVGLPELGPCᵢᵢSDLCᵢᵢᵢ; |
| (SEQ ID NO: 253) | |
| | CᵢVGL[tBuAla]ELGPCᵢᵢSDLCᵢᵢᵢ; |
| (SEQ ID NO: 254) | |
| | CᵢVGL[Cba]ELGPCᵢᵢSDLCᵢᵢᵢ; |
| (SEQ ID NO: 255) | |
| | CᵢVGL[Cha]ELGPCᵢᵢSDLCᵢᵢᵢ; |
| (SEQ ID NO: 256) | |
| | CᵢVGL[Nle]ELGPCᵢᵢSDLCᵢᵢᵢ; |
| (SEQ ID NO: 257) | |
| | CᵢVGLE[Gla]LGPCᵢᵢSDLCᵢᵢᵢ; |
| (SEQ ID NO: 258) | |
| | CᵢVGLEELG[Aze]CᵢᵢSDLCᵢᵢᵢ; |
| (SEQ ID NO: 259) | |
| | CᵢVGLEELGPCᵢᵢSD[tBuAla]Cᵢᵢᵢ; |
| (SEQ ID NO: 260) | |
| | CᵢVGLEELGPCᵢᵢSD[Cba]Cᵢᵢᵢ; |
| (SEQ ID NO: 261) | |
| | CᵢVGLEELGPCᵢᵢSD[Cha]Cᵢᵢᵢ; |
| (SEQ ID NO: 262) | |
| | CᵢVG[HLeu]EELGPCᵢᵢSDLCᵢᵢᵢ; |
| (SEQ ID NO: 263) | |
| | CᵢVGL[HLeu]ELGPCᵢᵢSDLCᵢᵢᵢ; |
| | and |
| (SEQ ID NO: 264) | |
| | CᵢVGLE[HGlu]LGPCᵢᵢSDLCᵢᵢᵢ; |

wherein Cᵢ, Cᵢᵢ and Cᵢᵢᵢ represent first, second and third cysteine residues, respectively, and wherein dA represents D-Alanine, tBuAla represents β-t-Butyl-L-alanine, Cba represents cyclobutylalanine, 1NaI represents 1-napthylalanine, 2NaI represents 2-naphthylalanine, Cha represents 3-cyclohexyl-D-alanine, Ne represents norleucine, Gla represents γ-carboxyglutamic acid, Aze represents azetidine-2-carboxylic acid, HLeu represents homoleucine, HGlu represents L-2-aminoadipic acid, or a pharmaceutically acceptable salt thereof, in particular:
wherein the molecular scaffold is TBMT and the bicyclic peptide ligand additionally comprises N- and/or C-terminal additions and comprises an amino acid sequence which is selected from:
A-(SEQ ID NO: 62)-A (herein referred to as BCY16493);
Ac-(SEQ ID NO: 62) (herein referred to as BCY16495);
A-(SEQ ID NO: 62)-A-[K(PYA)] (herein referred to as BCY13886);
A-(SEQ ID NO: 244)-A-[K(PYA)] (herein referred to as BCY16165);
A-(SEQ ID NO: 245)-A (herein referred to as BCY16498);
A-(SEQ ID NO: 246)-A (herein referred to as BCY16499);
A-(SEQ ID NO: 247)-A (herein referred to as BCY16501);
A-(SEQ ID NO: 248)-A (herein referred to as BCY16502);
A-(SEQ ID NO: 249)-A (herein referred to as BCY16504);
A-(SEQ ID NO: 250)-A (herein referred to as BCY16505);
A-(SEQ ID NO: 251)-A (herein referred to as BCY16506);
A-(SEQ ID NO: 252)-A (herein referred to as BCY16507);
A-(SEQ ID NO: 253)-A (herein referred to as BCY16508);
A-(SEQ ID NO: 254)-A (herein referred to as BCY16509);
A-(SEQ ID NO: 255)-A (herein referred to as BCY16510);
A-(SEQ ID NO: 256)-A (herein referred to as BCY16511);
A-(SEQ ID NO: 257)-A (herein referred to as BCY16512);
A-(SEQ ID NO: 258)-A (herein referred to as BCY16520);
A-(SEQ ID NO: 259)-A (herein referred to as BCY16522);
A-(SEQ ID NO: 260)-A (herein referred to as BCY16523);
A-(SEQ ID NO: 261)-A (herein referred to as BCY16524);
A-(SEQ ID NO: 262)-A (herein referred to as BCY16527);
A-(SEQ ID NO: 263)-A (herein referred to as BCY16528); and
A-(SEQ ID NO: 264)-A (herein referred to as BCY16529),
wherein PYA represents pentynoic acid.

16. The peptide ligand as defined in any one of claims 1 to 15, wherein the pharmaceutically acceptable salt is selected from the free acid or the sodium, potassium, calcium or ammonium salt.

17. A multimeric binding complex which comprises at least two bicyclic peptide ligands as defined in any one of claims 1 to 16, wherein said peptide ligands may be the same or different.

18. The multimeric binding complex as defined in claim 17, which comprises an NKp46 homodimer selected from BCY15663, BCY15921, BCY15922, BCY15455, and BCY18043.

19. The multimeric binding complex as defined in claim 17, which comprises a CD16a homodimer selected from BCY15914, BCY15915, and BCY15916.

20. A pharmaceutical composition which comprises the peptide ligand as defined in any one of claims 1 to 16 or the multimeric binding complex as defined in any one of claims 17 to 19, in combination with one or more pharmaceutically acceptable excipients.

21. The peptide ligand as defined in any one of claims 1 to 16, or the multimeric binding complex as defined in any one of claims 17 to 19, or the pharmaceutical composition of claim 20, for use in preventing, suppressing or treating a disease or disorder mediated by natural killer (NK) cells, such as inflammatory disorders, autoimmune disease and cancer.


SEQUENCE LISTING (268 sequences)

SEQ ID NO: 1 - protein, 12 aa, Artificial
  Synthetic Peptide
CNLQKPCMKYPC

SEQ ID NO: 2 - protein, 12 aa, Artificial
  Synthetic Peptide
CNLQNPCMKFPC

SEQ ID NO: 3 - protein, 14 aa, Artificial
  Synthetic Peptide
CNLQAPCMQTGKVC

SEQ ID NO: 4 - protein, 14 aa, Artificial
  Synthetic Peptide
CNLQAPCMLTGKVC

SEQ ID NO: 5 - protein, 15 aa, Artificial
  Synthetic Peptide
CLLHDHCPNTHPKLC

SEQ ID NO: 6 - protein, 15 aa, Artificial
  Synthetic Peptide
CLLHEHCPNTHPKMC

SEQ ID NO: 7 - protein, 15 aa, Artificial
  Synthetic Peptide
CLLHDHCPNSHPEIC

SEQ ID NO: 8 - protein, 15 aa, Artificial
  Synthetic Peptide
CLLHDHCPNTHPIIC

SEQ ID NO: 9 - protein, 13 aa, Artificial
  Synthetic Peptide
CYLPDWLCGDEYC

SEQ ID NO: 10 - protein, 13 aa, Artificial
  Synthetic Peptide
CYLPDYLCGDEYC

SEQ ID NO: 11 - protein, 13 aa, Artificial
  Synthetic Peptide
CDLTTHNCQWGIC

SEQ ID NO: 12 - protein, 13 aa, Artificial
  Synthetic Peptide
CYLPDYLCGDEYC

SEQ ID NO: 13 - protein, 13 aa, Artificial
  Synthetic Peptide
CYLPDWLCGDEYC

SEQ ID NO: 14 - protein, 13 aa, Artificial
  Synthetic Peptide
  Xaa (2)..(2): Xaa is 2,6DiMeTyr
CXLPDYLCGDEYC

SEQ ID NO: 15 - protein, 13 aa, Artificial
  Synthetic Peptide
  Xaa (3)..(3): Xaa is tBuAla
CYXPDYLCGDEYC

SEQ ID NO: 16 - protein, 13 aa, Artificial
  Synthetic Peptide
  Xaa (3)..(3): Xaa is Cba
CYXPDYLCGDEYC

SEQ ID NO: 17 - protein, 13 aa, Artificial
  Synthetic Peptide
  Xaa (4)..(4): Xaa is HyP
CYLXDYLCGDEYC

SEQ ID NO: 18 - protein, 13 aa, Artificial
  Synthetic Peptide
  Xaa (4)..(4): Xaa is Aze
CYLXDYLCGDEYC

SEQ ID NO: 19 - protein, 13 aa, Artificial
  Synthetic Peptide
  Xaa (4)..(4): Xaa is Aib
CYLXDYLCGDEYC

SEQ ID NO: 20 - protein, 13 aa, Artificial
  Synthetic Peptide
  Xaa (6)..(6): Xaa is 1Nal
CYLPDXLCGDEYC

SEQ ID NO: 21 - protein, 13 aa, Artificial
  Synthetic Peptide
  Xaa (6)..(6): Xaa is 2Nal
CYLPDXLCGDEYC

SEQ ID NO: 22 - protein, 13 aa, Artificial
  Synthetic Peptide
  Xaa (6)..(6): Xaa is 3Pal
CYLPDXLCGDEYC

SEQ ID NO: 23 - protein, 13 aa, Artificial
  Synthetic Peptide
  Xaa (6)..(6): Xaa is 2,6DiMeTyr
CYLPDXLCGDEYC

SEQ ID NO: 24 - protein, 13 aa, Artificial
  Synthetic Peptide
  Xaa (6)..(6): Xaa is tBuAla
  misc_feature (7)..(7): Xaa can be any naturally occurring amino acid
CYLPDWXCGDEYC

SEQ ID NO: 25 - protein, 13 aa, Artificial
  Synthetic Peptide
  Xaa (7)..(7): Xaa is Cba
CYLPDWXCGDEYC

SEQ ID NO: 26 - protein, 13 aa, Artificial
  Synthetic Peptide
CYLPDYLCADEYC

SEQ ID NO: 27 - protein, 13 aa, Artificial
  Synthetic Peptide
  Xaa (12)..(12): Xaa is 2,6DiMeTyr
CYLPDYLCGDEXC

SEQ ID NO: 28 - protein, 13 aa, Artificial
  Synthetic Peptide
CALPDYLCGDEYC

SEQ ID NO: 29 - protein, 13 aa, Artificial
  Synthetic Peptide
CYAPDYLCGDEYC

SEQ ID NO: 30 - protein, 13 aa, Artificial
  Synthetic Peptide
CYLADYLCGDEYC

SEQ ID NO: 31 - protein, 13 aa, Artificial
  Synthetic Peptide
CYLPAYLCGDEYC

SEQ ID NO: 32 - protein, 13 aa, Artificial
  Synthetic Peptide
CYLPDALCGDEYC

SEQ ID NO: 33 - protein, 13 aa, Artificial
  Synthetic Peptide
CYLPDYACGDEYC

SEQ ID NO: 34 - protein, 13 aa, Artificial
  Synthetic Peptide
CYLPDYLCADEYC

SEQ ID NO: 35 - protein, 13 aa, Artificial
  Synthetic Peptide
CYLPDYLCGAEYC

SEQ ID NO: 36 - protein, 13 aa, Artificial
  Synthetic Peptide
CYLPDYLCGDAYC

SEQ ID NO: 37 - protein, 13 aa, Artificial
  Synthetic Peptide
CYLPDYLCGDEAC

SEQ ID NO: 38 - protein, 13 aa, Artificial
  Synthetic Peptide
  Xaa (3)..(3): Xaa is C5A
CYXPDYLCGDEYC

SEQ ID NO: 39 - protein, 13 aa, Artificial
  Synthetic Peptide
  Xaa (3)..(3): Xaa is Cha
CYXPDYLCGDEYC

SEQ ID NO: 40 - protein, 13 aa, Artificial
  Synthetic Peptide
  Xaa (3)..(3): Xaa is Cba
CYXPDYLCADEYC

SEQ ID NO: 41 - protein, 13 aa, Artificial
  Synthetic Peptide
  Xaa (6)..(6): Xaa is 4FPhe
CYLPDXLCGDEYC

SEQ ID NO: 42 - protein, 13 aa, Artificial
  Synthetic Peptide
  Xaa (6)..(6): Xaa is 4MeOPhe
CYLPDXLCGDEYC

SEQ ID NO: 43 - protein, 13 aa, Artificial
  Synthetic Peptide
  Xaa (6)..(6): Xaa is NO2Phe
CYLPDXLCGDEYC

SEQ ID NO: 44 - protein, 13 aa, Artificial
  Synthetic Peptide
  Xaa (6)..(6): Xaa is 4MePhe
CYLPDXLCGDEYC

SEQ ID NO: 45 - protein, 13 aa, Artificial
  Synthetic Peptide
  Xaa (6)..(6): Xaa is pCoPhe
CYLPDXLCGDEYC

SEQ ID NO: 46 - protein, 13 aa, Artificial
  Synthetic Peptide
  misc_feature (6)..(6): Xaa can be any naturally occurring amino acid
CYLPDXLCGDEYC

SEQ ID NO: 47 - protein, 13 aa, Artificial
  Synthetic Peptide
  Xaa (6)..(6): Xaa is 4tBuPhe
CYLPDXLCGDEYC

SEQ ID NO: 48 - protein, 13 aa, Artificial
  Synthetic Peptide
  Xaa (6)..(6): Xaa is CF3Phe
CYLPDXLCGDEYC

SEQ ID NO: 49 - protein, 13 aa, Artificial
  Synthetic Peptide
  Xaa (6)..(6): Xaa is 4CNPhe
CYLPDXLCGDEYC

SEQ ID NO: 50 - protein, 13 aa, Artificial
  Synthetic Peptide
  Xaa (6)..(6): Xaa is 4ClPhe
CYLPDXLCGDEYC

SEQ ID NO: 51 - protein, 13 aa, Artificial
  Synthetic Peptide
  Xaa (6)..(6): Xaa is HPhe
CYLPDXLCGDEYC

SEQ ID NO: 52 - protein, 13 aa, Artificial
  Synthetic Peptide
  Xaa (2)..(2): Xaa is 2,6DiMeTyr
CXLPDWLCGDEYC

SEQ ID NO: 53 - protein, 13 aa, Artificial
  Synthetic Peptide
  Xaa (3)..(3): Xaa is Cba
CYXPDWLCGDEYC

SEQ ID NO: 54 - protein, 13 aa, Artificial
  Synthetic Peptide
  Xaa (4)..(4): Xaa is Aze
CYLXDWLCGDEYC

SEQ ID NO: 55 - protein, 13 aa, Artificial
  Synthetic Peptide
CYLPDWLCADEYC

SEQ ID NO: 56 - protein, 13 aa, Artificial
  Synthetic Peptide
CSLPDYLCGDEYC

SEQ ID NO: 57 - protein, 13 aa, Artificial
  Synthetic Peptide
CDLTTHNCQWGIC

SEQ ID NO: 58 - protein, 13 aa, Artificial
  Synthetic Peptide
CDLTTHNCLWGVC

SEQ ID NO: 59 - protein, 13 aa, Artificial
  Synthetic Peptide
CNLQLHNCIWGVC

SEQ ID NO: 60 - protein, 12 aa, Artificial
  Synthetic Peptide
CPPTVYCHPLDC

SEQ ID NO: 61 - protein, 13 aa, Artificial
  Synthetic Peptide
CRWSVEDPCGAWC

SEQ ID NO: 62 - protein, 14 aa, Artificial
  Synthetic Peptide
CVGLEELGPCSDLC

SEQ ID NO: 63 - protein, 14 aa, Artificial
  Synthetic Peptide
CNLQQACLHTGKVC

SEQ ID NO: 64 - protein, 14 aa, Artificial
  Synthetic Peptide
CNLQAACMLTGKVC

SEQ ID NO: 65 - protein, 14 aa, Artificial
  Synthetic Peptide
CNLQAPCMATGKVC

SEQ ID NO: 66 - protein, 14 aa, Artificial
  Synthetic Peptide
CNLQAPCMLAGKVC

SEQ ID NO: 67 - protein, 14 aa, Artificial
  Synthetic Peptide
CNLQAPCMLTAKVC

SEQ ID NO: 68 - protein, 14 aa, Artificial
  Synthetic Peptide
CNLQAPCMLTGAVC

SEQ ID NO: 69 - protein, 14 aa, Artificial
  Synthetic Peptide
CNLQAPCMLTGKAC

SEQ ID NO: 70 - protein, 14 aa, Artificial
  Synthetic Peptide
  Xaa (3)..(3): Xaa is tBuAla
CNXQAPCMLTGKVC

SEQ ID NO: 71 - protein, 14 aa, Artificial
  Synthetic Peptide
  Xaa (6)..(6): Xaa is Sar
CNLQAXCMLTGKVC

SEQ ID NO: 72 - protein, 14 aa, Artificial
  Synthetic Peptide
  Xaa (6)..(6): Xaa is Cis-HyP
CNLQAXCMLTGKVC

SEQ ID NO: 73 - protein, 14 aa, Artificial
  Synthetic Peptide
  Xaa (6)..(6): Xaa is HyP
CNLQAXCMLTGKVC

SEQ ID NO: 74 - protein, 14 aa, Artificial
  Synthetic Peptide
  Xaa (6)..(6): Xaa is NMeAla
CNLQAXCMLTGKVC

SEQ ID NO: 75 - protein, 14 aa, Artificial
  Synthetic Peptide
CNLQAPCLLTGKVC

SEQ ID NO: 76 - protein, 14 aa, Artificial
  Synthetic Peptide
  Xaa (8)..(8): Xaa is Nle
CNLQAPCXLTGKVC

SEQ ID NO: 77 - protein, 14 aa, Artificial
  Synthetic Peptide
  Xaa (8)..(8): Xaa is Cba
CNLQAPCXLTGKVC

SEQ ID NO: 78 - protein, 14 aa, Artificial
  Synthetic Peptide
CNLQAPCMETGKVC

SEQ ID NO: 79 - protein, 14 aa, Artificial
  Synthetic Peptide
CNLQAPCMFTGKVC

SEQ ID NO: 80 - protein, 14 aa, Artificial
  Synthetic Peptide
CNLQAPCMYTGKVC

SEQ ID NO: 81 - protein, 14 aa, Artificial
  Synthetic Peptide
  Xaa (9)..(9): Xaa is Cba
CNLQAPCMXTGKVC

SEQ ID NO: 82 - protein, 14 aa, Artificial
  Synthetic Peptide
  Xaa (9)..(9): Xaa is tBuAla
CNLQAPCMXTGKVC

SEQ ID NO: 83 - protein, 14 aa, Artificial
  Synthetic Peptide
  Xaa (9)..(9): Xaa is Cha
CNLQAPCMXTGKVC

SEQ ID NO: 84 - protein, 14 aa, Artificial
  Synthetic Peptide
CNLQAPCMLTAKVC

SEQ ID NO: 85 - protein, 14 aa, Artificial
  Synthetic Peptide
CNLQAPCMLTGRVC

SEQ ID NO: 86 - protein, 14 aa, Artificial
  Synthetic Peptide
  Xaa (12)..(12): Xaa is Agb
CNLQAPCMLTGXVC

SEQ ID NO: 87 - protein, 14 aa, Artificial
  Synthetic Peptide
  Xaa (13)..(13): Xaa is tBuGly
CNLQAPCMLTGKXC

SEQ ID NO: 88 - protein, 14 aa, Artificial
  Synthetic Peptide
  Xaa (13)..(13): Xaa is Chg
CNLQAPCMLTGKXC

SEQ ID NO: 89 - protein, 14 aa, Artificial
  Synthetic Peptide
  Xaa (13)..(13): Xaa is Cbg
CNLQAPCMLTGKXC

SEQ ID NO: 90 - protein, 14 aa, Artificial
  Synthetic Peptide
  Xaa (12)..(12): Xaa is HArg
CNLQAPCMLTGXVC

SEQ ID NO: 91 - protein, 14 aa, Artificial
  Synthetic Peptide
  Xaa (6)..(6): Xaa is Aze
CNLQAXCMLTGKVC

SEQ ID NO: 92 - protein, 14 aa, Artificial
  Synthetic Peptide
  Xaa (6)..(6): Xaa is Pip
CNLQAXCMLTGKVC

SEQ ID NO: 93 - protein, 14 aa, Artificial
  Synthetic Peptide
CNLQAPCMVTGKVC

SEQ ID NO: 94 - protein, 14 aa, Artificial
  Synthetic Peptide
  Xaa (9)..(9): Xaa is Nle
CNLQAPCMXTGKVC

SEQ ID NO: 95 - protein, 14 aa, Artificial
  Synthetic Peptide
  Xaa (9)..(9): Xaa is Nva
CNLQAPCMXTGKVC

SEQ ID NO: 96 - protein, 14 aa, Artificial
  Synthetic Peptide
  Xaa (13)..(13): Xaa is AlloIle
CNLQAPCMLTGKXC

SEQ ID NO: 97 - protein, 14 aa, Artificial
  Synthetic Peptide
  Xaa (13)..(13): Xaa is EPA
CNLQAPCMLTGKXC

SEQ ID NO: 98 - protein, 14 aa, Artificial
  Synthetic Peptide
CNLQQPCMLTGKVC

SEQ ID NO: 99 - protein, 14 aa, Artificial
  Synthetic Peptide
  misc_feature (6)..(6): Xaa can be any naturally occurring amino acid
CNLQAXCMLTGKVC

SEQ ID NO: 100 - protein, 14 aa, Artificial
  Synthetic Peptide
CNLQAPCMHTGKVC

SEQ ID NO: 101 - protein, 14 aa, Artificial
  Synthetic Peptide
CNLQAPCLHTGKVC

SEQ ID NO: 102 - protein, 14 aa, Artificial
  Synthetic Peptide
CNLQQACMQTGKVC

SEQ ID NO: 103 - protein, 15 aa, Artificial
  Synthetic Peptide
CLLHDHCPNTHPKMC

SEQ ID NO: 104 - protein, 13 aa, Artificial
  Synthetic Peptide
CDLTTHNCIWGVC

SEQ ID NO: 105 - protein, 13 aa, Artificial
  Synthetic Peptide
CNLQLHNCIWGVC

SEQ ID NO: 106 - protein, 13 aa, Artificial
  Synthetic Peptide
CNLQLHNCQWGIC

SEQ ID NO: 107 - protein, 13 aa, Artificial
  Synthetic Peptide
  Xaa (2)..(2): Xaa is 3tBuTyr
CXLPDYLCGDEYC

SEQ ID NO: 108 - protein, 13 aa, Artificial
  Synthetic Peptide
  Xaa (6)..(6): Xaa is 3tBuTyr
CYLPDXLCGDEYC

SEQ ID NO: 109 - protein, 13 aa, Artificial
  Synthetic Peptide
  Xaa (12)..(12): Xaa is 3tBuTyr
CYLPDYLCGDEXC

SEQ ID NO: 110 - protein, 13 aa, Artificial
  Synthetic Peptide
  Xaa (2)..(2): Xaa is K(PYA)
CXLPDYLCGDEYC

SEQ ID NO: 111 - protein, 13 aa, Artificial
  Synthetic Peptide
  Xaa (5)..(5): Xaa is K(PYA)
CYLPXYLCGDEYC

SEQ ID NO: 112 - protein, 13 aa, Artificial
  Synthetic Peptide
  Xaa (9)..(9): Xaa is K(PYA)
CYLPDYLCXDEYC

SEQ ID NO: 113 - protein, 13 aa, Artificial
  Synthetic Peptide
  Xaa (11)..(11): Xaa is K(PYA)
CYLPDYLCGDXYC

SEQ ID NO: 114 - protein, 13 aa, Artificial
  Synthetic Peptide
  Xaa (12)..(12): Xaa is K(PYA)
CYLPDYLCGDEXC

SEQ ID NO: 115 - protein, 13 aa, Artificial
  Synthetic Peptide
  Xaa (9)..(9): Xaa is dK{PYA)
CYLPDYLCXDEYC

SEQ ID NO: 116 - protein, 13 aa, Artificial
  Synthetic Peptide
CALTTHNCQWGIC

SEQ ID NO: 117 - protein, 13 aa, Artificial
  Synthetic Peptide
CDLATHNCQWGIC

SEQ ID NO: 118 - protein, 13 aa, Artificial
  Synthetic Peptide
CDLTAHNCQWGIC

SEQ ID NO: 119 - protein, 13 aa, Artificial
  Synthetic Peptide
CDLTTHNCAWGIC

SEQ ID NO: 120 - protein, 13 aa, Artificial
  Synthetic Peptide
CDLTTHNCQWGAC

SEQ ID NO: 121 - protein, 13 aa, Artificial
  Synthetic Peptide
CNLTTHNCQWGIC

SEQ ID NO: 122 - protein, 13 aa, Artificial
  Synthetic Peptide
CDLQTHNCQWGIC

SEQ ID NO: 123 - protein, 13 aa, Artificial
  Synthetic Peptide
CDLTLHNCQWGIC

SEQ ID NO: 124 - protein, 13 aa, Artificial
  Synthetic Peptide
  Xaa (6)..(6): Xaa is His3Me
CDLTTXNCQWGIC

SEQ ID NO: 125 - protein, 13 aa, Artificial
  Synthetic Peptide
  Xaa (6)..(6): Xaa is 2Pal
CDLTTXNCQWGIC

SEQ ID NO: 126 - protein, 13 aa, Artificial
  Synthetic Peptide
  Xaa (6)..(6): Xaa is His1Me
CDLTTXNCQWGIC

SEQ ID NO: 127 - protein, 13 aa, Artificial
  Synthetic Peptide
  Xaa (6)..(6): Xaa is 4ThiAz
CDLTTXNCQWGIC

SEQ ID NO: 128 - protein, 13 aa, Artificial
  Synthetic Peptide
CDLTTHNCVWGIC

SEQ ID NO: 129 - protein, 13 aa, Artificial
  Synthetic Peptide
  misc_feature (9)..(9): Xaa can be any naturally occurring amino acid
CDLTTHNCXWGIC

SEQ ID NO: 130 - protein, 13 aa, Artificial
  Synthetic Peptide
  Xaa (10)..(10): Xaa is 1Nal
CDLTTHNCQXGIC

SEQ ID NO: 131 - protein, 13 aa, Artificial
  Synthetic Peptide
  Xaa (10)..(10): Xaa is Trp(Me)
CDLTTHNCQXGIC

SEQ ID NO: 132 - protein, 13 aa, Artificial
  Synthetic Peptide
  Xaa (12)..(12): Xaa is tBuGly
CDLTTHNCQWGXC

SEQ ID NO: 133 - protein, 13 aa, Artificial
  Synthetic Peptide
  Xaa (12)..(12): Xaa is tBuAla
CDLTTHNCQWGXC

SEQ ID NO: 134 - protein, 13 aa, Artificial
  Synthetic Peptide
  Xaa (12)..(12): Xaa is Chg
CDLTTHNCQWGXC

SEQ ID NO: 135 - protein, 13 aa, Artificial
  Synthetic Peptide
CDLTTHNCQWAIC

SEQ ID NO: 136 - protein, 13 aa, Artificial
  Synthetic Peptide
  Xaa (3)..(3): Xaa is Cba
CSXPDYLCADEYC

SEQ ID NO: 137 - protein, 13 aa, Artificial
  Synthetic Peptide
  Xaa (2)..(2): Xaa is HSer
  Xaa (3)..(3): Xaa is Cba
CXXPDYLCADEYC

SEQ ID NO: 138 - protein, 13 aa, Artificial
  Synthetic Peptide
  Xaa (3)..(3): Xaa is Cba
CNXPDYLCADEYC

SEQ ID NO: 139 - protein, 13 aa, Artificial
  Synthetic Peptide
  Xaa (2)..(2): Xaa is 4Pal
  Xaa (3)..(3): Xaa is Cba
CXXPDYLCADEYC

SEQ ID NO: 140 - protein, 13 aa, Artificial
  Synthetic Peptide
  Xaa (3)..(3): Xaa is Cba
  Xaa (4)..(4): Xaa is Cis-HyP
CYXXDYLCADEYC

SEQ ID NO: 141 - protein, 13 aa, Artificial
  Synthetic Peptide
  Xaa (3)..(3): Xaa is Cba
  Xaa (4)..(4): Xaa is NMeAla
CYXXDYLCADEYC

SEQ ID NO: 142 - protein, 13 aa, Artificial
  Synthetic Peptide
  Xaa (3)..(3): Xaa is Cba
CYXPNYLCADEYC

SEQ ID NO: 143 - protein, 13 aa, Artificial
  Synthetic Peptide
  Xaa (3)..(3): Xaa is Cba
CYXPQYLCADEYC

SEQ ID NO: 144 - protein, 13 aa, Artificial
  Synthetic Peptide
  Xaa (3)..(3): Xaa is Cba
CYXPDYLCVDEYC

SEQ ID NO: 145 - protein, 13 aa, Artificial
  Synthetic Peptide
  Xaa (3)..(3): Xaa is Cba
  Xaa (9)..(9): Xaa is dNva
CYXPDYLCXDEYC

SEQ ID NO: 146 - protein, 13 aa, Artificial
  Synthetic Peptide
  Xaa (3)..(3): Xaa is Cba
CYXPDYLCFDEYC

SEQ ID NO: 147 - protein, 13 aa, Artificial
  Synthetic Peptide
  Xaa (3)..(3): Xaa is Cba
  Xaa (9)..(9): Xaa is dCha
CYXPDYLCXDEYC

SEQ ID NO: 148 - protein, 13 aa, Artificial
  Synthetic Peptide
  Xaa (3)..(3): Xaa is Cba
  Xaa (9)..(9): Xaa is dK(PYA)
CYXPDYLCXDEYC

SEQ ID NO: 149 - protein, 13 aa, Artificial
  Synthetic Peptide
  Xaa (3)..(3): Xaa is Cba
  Xaa (9)..(9): Xaa is dNle
CYXPDYLCXDEYC

SEQ ID NO: 150 - protein, 13 aa, Artificial
  Synthetic Peptide
  Xaa (3)..(3): Xaa is Cba
  Xaa (6)..(6): Xaa is DOPA
CYXPDXLCADEYC

SEQ ID NO: 151 - protein, 13 aa, Artificial
  Synthetic Peptide
  Xaa (3)..(3): Xaa is Cba
  Xaa (6)..(6): Xaa is 2FTyr
CYXPDXLCADEYC

SEQ ID NO: 152 - protein, 13 aa, Artificial
  Synthetic Peptide
  Xaa (3)..(3): Xaa is Cba
  Xaa (7)..(7): Xaa is Nle
CYXPDYXCADEYC

SEQ ID NO: 153 - protein, 13 aa, Artificial
  Synthetic Peptide
  Xaa (1)..(1): Xaa is Pen
  Xaa (3)..(3): Xaa is Cba
XYXPDYLCADEYC

SEQ ID NO: 154 - protein, 13 aa, Artificial
  Synthetic Peptide
  Xaa (3)..(3): Xaa is Cba
  Xaa (8)..(8): Xaa is Pen
CYXPDYLXADEYC

SEQ ID NO: 155 - protein, 13 aa, Artificial
  Synthetic Peptide
  Xaa (3)..(3): Xaa is Cba
  Xaa (13)..(13): Xaa is Pen
CYXPDYLCADEYX

SEQ ID NO: 156 - protein, 13 aa, Artificial
  Synthetic Peptide
  Xaa (3)..(3): Xaa is Cba
CYXPDYLCADEYC

SEQ ID NO: 157 - protein, 13 aa, Artificial
  Synthetic Peptide
  Xaa (3)..(3): Xaa is Cba
CYXPDYLCADEYC

SEQ ID NO: 158 - protein, 13 aa, Artificial
  Synthetic Peptide
  Xaa (10)..(10): Xaa is AzaTrp
CDLTTHNCQXGIC

SEQ ID NO: 159 - protein, 13 aa, Artificial
  Synthetic Peptide
  Xaa (10)..(10): Xaa is 2MeTrp
CDLTTHNCQXGIC

SEQ ID NO: 160 - protein, 13 aa, Artificial
  Synthetic Peptide
  Xaa (10)..(10): Xaa is 6MeTrp
CDLTTHNCQXGIC

SEQ ID NO: 161 - protein, 13 aa, Artificial
  Synthetic Peptide
  Xaa (10)..(10): Xaa is 7MeTrp
CDLTTHNCQXGIC

SEQ ID NO: 162 - protein, 13 aa, Artificial
  Synthetic Peptide
  Xaa (10)..(10): Xaa is 6FTrp
CDLTTHNCQXGIC

SEQ ID NO: 163 - protein, 13 aa, Artificial
  Synthetic Peptide
  Xaa (10)..(10): Xaa is 5FTrp
CDLTTHNCQXGIC

SEQ ID NO: 164 - protein, 13 aa, Artificial
  Synthetic Peptide
  Xaa (10)..(10): Xaa is Dht
CDLTTHNCQXGIC

SEQ ID NO: 165 - protein, 13 aa, Artificial
  Synthetic Peptide
  Xaa (6)..(6): Xaa is 2FuAla
CDLTTXNCQWGIC

SEQ ID NO: 166 - protein, 13 aa, Artificial
  Synthetic Peptide
  Xaa (12)..(12): Xaa is EPA
CDLTTHNCQWGXC

SEQ ID NO: 167 - protein, 13 aa, Artificial
  Synthetic Peptide
  Xaa (12)..(12): Xaa is Nle
CDLTTHNCQWGXC

SEQ ID NO: 168 - protein, 13 aa, Artificial
  Synthetic Peptide
  Xaa (12)..(12): Xaa is AlloIle
CDLTTHNCQWGXC

SEQ ID NO: 169 - protein, 12 aa, Artificial
  Synthetic Peptide
CPPAVYCHPLDC

SEQ ID NO: 170 - protein, 12 aa, Artificial
  Synthetic Peptide
CPPEVFCHPLDC

SEQ ID NO: 171 - protein, 12 aa, Artificial
  Synthetic Peptide
CPPEVYCHPLDC

SEQ ID NO: 172 - protein, 12 aa, Artificial
  Synthetic Peptide
CPPEVWCHPLDC

SEQ ID NO: 173 - protein, 12 aa, Artificial
  Synthetic Peptide
CPPQVYCHPLDC

SEQ ID NO: 174 - protein, 12 aa, Artificial
  Synthetic Peptide
CPPEVWCHPLDC

SEQ ID NO: 175 - protein, 13 aa, Artificial
  Synthetic Peptide
  Xaa (5)..(5): Xaa is tBuGly
CRWSXEDPCGAWC

SEQ ID NO: 176 - protein, 13 aa, Artificial
  Synthetic Peptide
  Xaa (8)..(8): Xaa is HyP
CRWSVEDXCGAWC

SEQ ID NO: 177 - protein, 13 aa, Artificial
  Synthetic Peptide
  Xaa (8)..(8): Xaa is Aze
CRWSVEDXCGAWC

SEQ ID NO: 178 - protein, 13 aa, Artificial
  Synthetic Peptide
CRWSVEDPCAAWC

SEQ ID NO: 179 - protein, 13 aa, Artificial
  Synthetic Peptide
  Xaa (7)..(7): Xaa is Gla
CRWSVEXPCGAWC

SEQ ID NO: 180 - protein, 13 aa, Artificial
  Synthetic Peptide
  Xaa (8)..(8): Xaa is Aze
CRWSVEDXCGAWC

SEQ ID NO: 181 - protein, 13 aa, Artificial
  Synthetic Peptide
CMWIRSDPCGAWC

SEQ ID NO: 182 - protein, 13 aa, Artificial
  Synthetic Peptide
CRWYPDDPCGAWC

SEQ ID NO: 183 - protein, 13 aa, Artificial
  Synthetic Peptide
CEWHISEPCGAWC

SEQ ID NO: 184 - protein, 13 aa, Artificial
  Synthetic Peptide
CNWHVYEPCGAWC

SEQ ID NO: 185 - protein, 13 aa, Artificial
  Synthetic Peptide
CRWHFSEPCGAWC

SEQ ID NO: 186 - protein, 13 aa, Artificial
  Synthetic Peptide
CRWNLDDPCGAWC

SEQ ID NO: 187 - protein, 13 aa, Artificial
  Synthetic Peptide
CMWIRSDPCGAWC

SEQ ID NO: 188 - protein, 13 aa, Artificial
  Synthetic Peptide
CRWYPDDPCGAWC

SEQ ID NO: 189 - protein, 13 aa, Artificial
  Synthetic Peptide
CEWHISEPCGAWC

SEQ ID NO: 190 - protein, 13 aa, Artificial
  Synthetic Peptide
CNWHVYEPCGAWC

SEQ ID NO: 191 - protein, 13 aa, Artificial
  Synthetic Peptide
CRWHFSEPCGAWC

SEQ ID NO: 192 - protein, 13 aa, Artificial
  Synthetic Peptide
CRWNLDDPCGAWC

SEQ ID NO: 193 - protein, 13 aa, Artificial
  Synthetic Peptide
  Xaa (3)..(3): Xaa is 2MeTrp
CRXSVEDPCGAWC

SEQ ID NO: 194 - protein, 13 aa, Artificial
  Synthetic Peptide
  Xaa (3)..(3): Xaa is 6MeTrp
CRXSVEDPCGAWC

SEQ ID NO: 195 - protein, 13 aa, Artificial
  Synthetic Peptide
  Xaa (3)..(3): Xaa is 6FTrp
CRXSVEDPCGAWC

SEQ ID NO: 196 - protein, 13 aa, Artificial
  Synthetic Peptide
  Xaa (3)..(3): Xaa is 5FTrp
CRXSVEDPCGAWC

SEQ ID NO: 197 - protein, 13 aa, Artificial
  Synthetic Peptide
  Xaa (3)..(3): Xaa is 6ClTrp
CRXSVEDPCGAWC

SEQ ID NO: 198 - protein, 13 aa, Artificial
  Synthetic Peptide
  Xaa (3)..(3): Xaa is Trp(S)
CRXSVEDPCGAWC

SEQ ID NO: 199 - protein, 13 aa, Artificial
  Synthetic Peptide
  Xaa (3)..(3): Xaa is AzaTrp
CRXSVEDPCGAWC

SEQ ID NO: 200 - protein, 13 aa, Artificial
  Synthetic Peptide
  Xaa (12)..(12): Xaa is 2MeTrp
CRWSVEDPCGAXC

SEQ ID NO: 201 - protein, 13 aa, Artificial
  Synthetic Peptide
  Xaa (12)..(12): Xaa is 6FTrp
CRWSVEDPCGAXC

SEQ ID NO: 202 - protein, 13 aa, Artificial
  Synthetic Peptide
  Xaa (12)..(12): Xaa is 5FTrp
CRWSVEDPCGAXC

SEQ ID NO: 203 - protein, 13 aa, Artificial
  Synthetic Peptide
  Xaa (12)..(12): Xaa is 4MeoTrp
CRWSVEDPCGAXC

SEQ ID NO: 204 - protein, 13 aa, Artificial
  Synthetic Peptide
  Xaa (12)..(12): Xaa is 5MeoTrp
CRWSVEDPCGAXC

SEQ ID NO: 205 - protein, 13 aa, Artificial
  Synthetic Peptide
  Xaa (12)..(12): Xaa is Trp(S)
CRWSVEDPCGAXC

SEQ ID NO: 206 - protein, 13 aa, Artificial
  Synthetic Peptide
  Xaa (12)..(12): Xaa is DOPA
CRWSVEDPCGAXC

SEQ ID NO: 207 - protein, 13 aa, Artificial
  Synthetic Peptide
CRWYPDDPCGAWC

SEQ ID NO: 208 - protein, 13 aa, Artificial
  Synthetic Peptide
CRWHFSEPCGAWC

SEQ ID NO: 209 - protein, 13 aa, Artificial
  Synthetic Peptide
  Xaa (3)..(3): Xaa is 2MeTrp
CRXHFSEPCGAWC

SEQ ID NO: 210 - protein, 13 aa, Artificial
  Synthetic Peptide
CRWYFSEPCGAWC

SEQ ID NO: 211 - protein, 13 aa, Artificial
  Synthetic Peptide
CRWSFSEPCGAWC

SEQ ID NO: 212 - protein, 13 aa, Artificial
  Synthetic Peptide
CRWHPSEPCGAWC

SEQ ID NO: 213 - protein, 13 aa, Artificial
  Synthetic Peptide
CRWYPSEPCGAWC

SEQ ID NO: 214 - protein, 13 aa, Artificial
  Synthetic Peptide
CRWHPDEPCGAWC

SEQ ID NO: 215 - protein, 13 aa, Artificial
  Synthetic Peptide
CRWHPEEPCGAWC

SEQ ID NO: 216 - protein, 13 aa, Artificial
  Synthetic Peptide
CRWHFDEPCGAWC

SEQ ID NO: 217 - protein, 13 aa, Artificial
  Synthetic Peptide
CRWHFDDPCGAWC

SEQ ID NO: 218 - protein, 13 aa, Artificial
  Synthetic Peptide
CRWYFDEPCGAWC

SEQ ID NO: 219 - protein, 13 aa, Artificial
  Synthetic Peptide
  Xaa (4)..(4): Xaa is His1Me
CRWXFSEPCGAWC

SEQ ID NO: 220 - protein, 13 aa, Artificial
  Synthetic Peptide
  Xaa (4)..(4): Xaa is His3Me
CRWXFSEPCGAWC

SEQ ID NO: 221 - protein, 13 aa, Artificial
  Synthetic Peptide
  Xaa (4)..(4): Xaa is 4MeoPhe
CRWXFSEPCGAWC

SEQ ID NO: 222 - protein, 13 aa, Artificial
  Synthetic Peptide
  Xaa (4)..(4): Xaa is DOPA
CRWXFSEPCGAWC

SEQ ID NO: 223 - protein, 13 aa, Artificial
  Synthetic Peptide
  Xaa (4)..(4): Xaa is 26DiMeTyr
CRWXFSEPCGAWC

SEQ ID NO: 224 - protein, 13 aa, Artificial
  Synthetic Peptide
  Xaa (4)..(4): Xaa is 3BuTyr
CRWXFSEPCGAWC

SEQ ID NO: 225 - protein, 13 aa, Artificial
  Synthetic Peptide
  Xaa (4)..(4): Xaa is 4FPhe
CRWXFSEPCGAWC

SEQ ID NO: 226 - protein, 13 aa, Artificial
  Synthetic Peptide
  Xaa (5)..(5): Xaa is 4MePhe
CRWHXSEPCGAWC

SEQ ID NO: 227 - protein, 13 aa, Artificial
  Synthetic Peptide
  Xaa (5)..(5): Xaa is Aze
CRWHXSEPCGAWC

SEQ ID NO: 228 - protein, 13 aa, Artificial
  Synthetic Peptide
  Xaa (5)..(5): Xaa is HyP
CRWHXSEPCGAWC

SEQ ID NO: 229 - protein, 13 aa, Artificial
  Synthetic Peptide
  Xaa (5)..(5): Xaa is 4tBuPhe
CRWHXSEPCGAWC

SEQ ID NO: 230 - protein, 13 aa, Artificial
  Synthetic Peptide
  Xaa (5)..(5): Xaa is HPhe
CRWHXSEPCGAWC

SEQ ID NO: 231 - protein, 13 aa, Artificial
  Synthetic Peptide
  Xaa (5)..(5): Xaa is 4PhePro
CRWHXSEPCGAWC

SEQ ID NO: 232 - protein, 13 aa, Artificial
  Synthetic Peptide
  Xaa (6)..(6): Xaa is HSer
CRWHFXEPCGAWC

SEQ ID NO: 233 - protein, 13 aa, Artificial
  Synthetic Peptide
  Xaa (7)..(7): Xaa is Gla
CRWHFSXPCGAWC

SEQ ID NO: 234 - protein, 13 aa, Artificial
  Synthetic Peptide
  Xaa (7)..(7): Xaa is HGlu
CRWHFSXPCGAWC

SEQ ID NO: 235 - protein, 13 aa, Artificial
  Synthetic Peptide
CRWHFSEPCAAWC

SEQ ID NO: 236 - protein, 13 aa, Artificial
  Synthetic Peptide
  Xaa (8)..(8): Xaa is Aze
CRWYFSEXCAAWC

SEQ ID NO: 237 - protein, 13 aa, Artificial
  Synthetic Peptide
  Xaa (8)..(8): Xaa is Aze
CRWHFDEXCAAWC

SEQ ID NO: 238 - protein, 13 aa, Artificial
  Synthetic Peptide
  Xaa (8)..(8): Xaa is Aze
CRWYPSEXCAAWC

SEQ ID NO: 239 - protein, 13 aa, Artificial
  Synthetic Peptide
  Xaa (2)..(2): Xaa is HArg
CXWHFSEPCGAWC

SEQ ID NO: 240 - protein, 13 aa, Artificial
  Synthetic Peptide
  Xaa (5)..(5): Xaa is Cis-HyP
CRWHXSEPCGAWC

SEQ ID NO: 241 - protein, 13 aa, Artificial
  Synthetic Peptide
CRWHFSEPCGAWC

SEQ ID NO: 242 - protein, 13 aa, Artificial
  Synthetic Peptide
CRWHFSEPCGAWC

SEQ ID NO: 243 - protein, 13 aa, Artificial
  Synthetic Peptide
CRWHFSEPCGAWC

SEQ ID NO: 244 - protein, 14 aa, Artificial
  Synthetic Peptide
CVGLLELGPCSDLC

SEQ ID NO: 245 - protein, 14 aa, Artificial
  Synthetic Peptide
CPGLEELGPCSDLC

SEQ ID NO: 246 - protein, 14 aa, Artificial
  Synthetic Peptide
CVALEELGPCSDLC

SEQ ID NO: 247 - protein, 14 aa, Artificial
  Synthetic Peptide
  Xaa (4)..(4): Xaa is tBuAla
CVGXEELGPCSDLC

SEQ ID NO: 248 - protein, 14 aa, Artificial
  Synthetic Peptide
  Xaa (4)..(4): Xaa is Cba
CVGXEELGPCSDLC

SEQ ID NO: 249 - protein, 14 aa, Artificial
  Synthetic Peptide
CVGFEELGPCSDLC

SEQ ID NO: 250 - protein, 14 aa, Artificial
  Synthetic Peptide
  Xaa (4)..(4): Xaa is 1Nal
CVGXEELGPCSDLC

SEQ ID NO: 251 - protein, 14 aa, Artificial
  Synthetic Peptide
  Xaa (4)..(4): Xaa is 2Nal
CVGXEELGPCSDLC

SEQ ID NO: 252 - protein, 14 aa, Artificial
  Synthetic Peptide
CVGLPELGPCSDLC

SEQ ID NO: 253 - protein, 14 aa, Artificial
  Synthetic Peptide
  Xaa (5)..(5): Xaa is tBuAla
CVGLXELGPCSDLC

SEQ ID NO: 254 - protein, 14 aa, Artificial
  Synthetic Peptide
  Xaa (5)..(5): Xaa is Cba
CVGLXELGPCSDLC

SEQ ID NO: 255 - protein, 14 aa, Artificial
  Synthetic Peptide
  Xaa (5)..(5): Xaa is Cha
CVGLXELGPCSDLC

SEQ ID NO: 256 - protein, 14 aa, Artificial
  Synthetic Peptide
  Xaa (5)..(5): Xaa is Nle
CVGLXELGPCSDLC

SEQ ID NO: 257 - protein, 14 aa, Artificial
  Synthetic Peptide
  Xaa (6)..(6): Xaa is Gla
CVGLEXLGPCSDLC

SEQ ID NO: 258 - protein, 14 aa, Artificial
  Synthetic Peptide
  Xaa (9)..(9): Xaa is Aze
CVGLEELGXCSDLC

SEQ ID NO: 259 - protein, 14 aa, Artificial
  Synthetic Peptide
  Xaa (13)..(13): Xaa is tBuAla
CVGLEELGPCSDXC

SEQ ID NO: 260 - protein, 14 aa, Artificial
  Synthetic Peptide
  Xaa (13)..(13): Xaa is Cba
CVGLEELGPCSDXC

SEQ ID NO: 261 - protein, 14 aa, Artificial
  Synthetic Peptide
  Xaa (13)..(13): Xaa is Cha
CVGLEELGPCSDXC

SEQ ID NO: 262 - protein, 14 aa, Artificial
  Synthetic Peptide
  Xaa (4)..(4): Xaa is HLeu
CVGXEELGPCSDLC

SEQ ID NO: 263 - protein, 14 aa, Artificial
  Synthetic Peptide
  Xaa (5)..(5): Xaa is HLeu
CVGLXELGPCSDLC

SEQ ID NO: 264 - protein, 14 aa, Artificial
  Synthetic Peptide
  Xaa (6)..(6): Xaa is HGlu
CVGLEXLGPCSDLC

SEQ ID NO: 265 - protein, 15 aa, Artificial
  Synthetic Peptide
CTWPYCKSWGDVQDC

SEQ ID NO: 266 - protein, 12 aa, Artificial
  Synthetic Peptide
CDWWDPGCTYFC

SEQ ID NO: 267 - protein, 13 aa, Artificial
  Synthetic Peptide
  Xaa (5)..(5): Xaa is 4BnPro
CRWHXSEPCGAWC

SEQ ID NO: 268 - protein, 13 aa, Artificial
  Synthetic Peptide
  Xaa (5)..(5): Xaa is HPhe
  Xaa (8)..(8): Xaa is Aze
CRWHXSEXCGAWC